(19) United States
(12) Patent Application Publication
(10) Pub. No.: US 2010/0003250 A1
(43) Pub. Date: Jan. 7, 2010

(54) (2-ARYL-7H-PYRROLO[2,3-D]PYRIMIDIN-4-YL)MORPHOLINE COMPOUNDS, THEIR USE AS MTOR KINASE AND PI3 KINASE INHIBITORS, AND THEIR SYNTHESES

(75) Inventors: Zecheng Chen, New City, NY (US); Aranapakam Mudumbai Venkatesan, Rego Park, NY (US); Arie Zask, New York, NY (US); Jeroen Cunera Verheijen, Highland Mills, NY (US); Semiramis Ayral-Kaloustian, Tarrytown, NY (US); Tarek Suhayl Mansour, New City, NY (US); Kevin Joseph Curran, Congers, NY (US)

Correspondence Address: WYETH;PATENT LAW GROUP, 5 GIRALDA FARMS, MADISON, NJ, 07940, US

(73) Assignee: Wyeth, Madison, NJ (US)

(21) Appl. No.: 12/496,675

(22) Filed: Jul. 2, 2009

Related U.S. Application Data
(60) Provisional application No. 61/077,595, filed on Jul. 2, 2008.

(51) Int. Cl. A61K 31/5377; C07D 487/04; A61K 39/395; A61P 35/00

(52) U.S. Cl. 424/133.1; 544/117; 514/234.2

(57) ABSTRACT

The invention relates to 2-aryl-7H-pyrrolo[2,3-d]pyrimidin-4-yl)morpholine compounds of the Formula I:

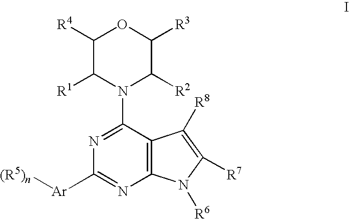

or a pharmaceutically acceptable salt thereof, wherein the constituent variables are as defined herein, compositions comprising the compounds, and methods for making and using the compounds.

### FIELD OF THE INVENTION

The invention relates to 2-aryl-7H-pyrrolo[2,3-d]pyrimidin-4-yl)morpholine compounds, compositions comprising them, methods of for their synthesis, and methods for treating mTOR-related diseases and PI3K-related diseases comprising the administration of an effective amount of a 2-aryl-7H-pyrrolo[2,3-d]pyrimidin-4-yl)morpholine compound.

### BACKGROUND OF THE INVENTION

Phosphatidylinositol (hereinafter abbreviated as “PI”) is one of the phospholipids in cell membranes. In recent years it has become clear that PI plays an important role also in intracellular signal transduction. It is well recognized in the art that PI (4,5) bisphosphate (PI(4,5)P2 or PIP2) is degraded into diacylglycerol and inositol (1,4,5) triphosphate by phospholipase C to induce activation of protein kinase C and intracellular calcium mobilization, respectively [M. J. Berridge et al., Nature, 312, 315 (1984); Y. Nishizuka, Science, 225, 1365 (1984)].

In the late 1980s, phosphatidylinositol-3 kinase (“PI3K”) was found to be an enzyme that phosphorylates the 3-position of the inositol ring of phosphatidylinositol [D. Whitman et al., Nature, 332, 664 (1988)]. When PI3K was discovered, it was originally considered to be a single enzyme. Recently however, it was clarified that a plurality of PI3K subtypes exists. Three major subtypes of PI3Ks have now been identified on the basis of their in vitro substrate specificity, and these three are designated class I (a & b), class II, and class III [B. Vanhaesebroeck, Trend in Biol. Sci., 22, 267(1997)].

The class Ia PI3K subtype has been most extensively investigated to date. Within the class Ia subtype there are three isoforms (α, β, & δ) that exist as hetero dimers of a catalytic 110-kDa subunit and regulatory subunits of 50-85 kDa. The regulatory subunits contain SH2 domains that bind to phosphorylated tyrosine residues within growth factor receptors or adaptor molecules and thereby localize PI3K to the inner cell membrane. At the inner cell membrane PI3K converts PIP2 to PIP3 (phosphatidylinositol-3,4,5-trisphosphate) that serves to localize the downstream effectors PDK1 and Akt to the inner cell membrane where Akt activation occurs. Activated Akt mediates a diverse array of effects including inhibition of apoptosis, cell cycle progression, response to insulin signaling, and cell proliferation. Class Ia PI3K subtypes also contain Ras binding domains (RBD) that allow association with activated Ras providing another mechanism for PI3K membrane localization. Activated, oncogenic forms of growth factor receptors, Ras, and even PI3K kinase have been shown to aberrantly elevate signaling in the PI3K/Akt/mTOR pathway resulting in cell transformation. As a central component of the PI3K/Akt/mTOR signaling pathway PI3K (particularly the class Ia α isoform) has become a major therapeutic target in cancer drug discovery.

Substrates for class I PI3Ks are PI, PI(4)P and PI(4,5)P2, with PI(4,5)P2 being the most favored. Class I PI3Ks are further divided into two groups, class Ia and class Ib, because of their activation mechanism and associated regulatory subunits. The class Ib PI3K is p110γ that is activated by interaction with G protein-coupled receptors. Interaction between p110γ and G protein-coupled receptors is mediated by regulatory subunits of 110, 87, and 84 kDa.

PI and PI(4)P are the known substrates for class II PI3Ks; PI(4,5)P2 is not a substrate for the enzymes of this class. Class II PI3Ks include PI3K C2α, C2β and C2γ isoforms, which contain C2 domains at the C terminus, implying that their activity is regulated by calcium ions.

The substrate for class III PI3Ks is PI only. A mechanism for activation of the class III PI3Ks has not been clarified. Because each subtype has its own mechanism for regulating activity, it is likely that activation mechanism(s) depend on stimuli specific to each respective class of PI3K.

The compound PI103 (3-(4-(4-morpholinyl)pyrido[3′,2′:4,5]furo[3,2-d]pyrimidin-2-yl)phenol) inhibits PI3K_{α} and PI3K_{γ} as well as the mTOR complexes with IC₅₀ values of 2, 3, and 50-80 nM respectively. I.P. dosing in mice of this compound in human tumor xenograft models of cancer demonstrated activity against a number of human tumor models, including the glioblastoma (PTEN null U87MG), prostate (PC3), breast (MDA-MB-468 and MDA-MB-435) colon carcinoma (HCT 116); and ovarian carcinoma (SKOV3 and IGROV-1); (Raynaud et al, Pharmacologic Characterization of a Potent Inhibitor of Class I Phosphatidylinositide 3-Kinases, Cancer Res. 2007 67: 5840-5850).

The compound ZSTK474 (2-(2-difluoromethylbenzoimidazol-1-yl)-4,6-dimorpholino-1,3,5-triazine) inhibits PI3K_{α} and PI3K_{γ} but not the mTOR enzymes with IC₅₀ values of 16, 4.6 and >10,000 nM respectively (Dexin Kong and Takao Yamori, ZSTK474 is an ATP-competitive inhibitor of class I phosphatidylinositol 3 kinase isoforms, Cancer Science, 2007, 98:10 1638-1642). Chronic oral administration of ZSTK474 in mouse human xenograft cancer models, completely inhibited growth that originated from a non-small-cell lung cancer (A549), a prostate cancer (PC-3), and a colon cancer (WiDr) at a dose of 400 mg/kg. (Yaguchi et al, Antitumor Activity of ZSTK474, a New Phosphatidylinositol 3-Kinase Inhibitor, J. Natl. Cancer Inst. 98: 545-556).

The compound NVP-BEZ-235 (2-methyl-2-(4-(3-methyl-2-oxo-8-(quinolin-3-yl)-2,3-dihydro-1H-imidazo[4,5-c]quinolin-1-yl)phenyl)propanenitrile) inhibits both PI3K_{α} and PI3K_{γ} as well as the mTOR enzyme with IC₅₀ values 4, 5, and “nanomolar”. Testing in human tumor xenograft models of cancer demonstrated activity against human tumor models of prostrate (PC-3) and glioblastoma (U-87) cancer. It entered clinical trials in December of 2006 (Verheijen, J. C. and Zask, A., Phosphatidylinositol 3-kinase (PI3K) inhibitors as anticancer drugs, Drugs Fut. 2007, 32(6): 537-547).

The compound SF-1126 (a prodrug form of LY-294002, which is 2-(4-morpholinyl)-8-phenyl-4H-1-benzopyran-4-one) is “a pan-PI3K inhibitor”. It is active in preclinical mouse cancer models of prostrate, breast, ovarian, lung, multiple myeloma, and brain cancers. It began clinical trials in April, 2007 for the solid tumors endometrial, renal cell, breast, hormone refractory prostate and ovarian cancers. (Verheijen, J. C. and Zask, A., Phosphatidylinositol 3-kinase (PI3K) inhibitors as anticancer drugs, Drugs Fut. 2007, 32(6): 537-547).

Exelixis Inc. (So. San Francisco, Calif.) recently filed INDs for XL-147 (a selective pan-PI3K inhibitor of unknown structure) and XL-765 (a mixed inhibitor of mTOR and PI3K of unknown structure) as anticancer agents. TargeGen's short-acting mixed inhibitor of PI3K_{γ} and δ, TG-100115, is in phase I/II trials for treatment of infarct following myocardial ischemia-reperfusion injury. Cerylid's antithrombotic PI3Kβ inhibitor CBL-1309 (structure unknown) has completed preclinical toxicology studies.

According to Verheijen, J. C. and Zask, A., Phosphatidylinositol 3-kinase (PI3K) inhibitors as anticancer drugs, Drugs Fut. 2007, 32(6): 537-547,
Although it seems clear that inhibition of the α isoform is essential for the antitumor activity of PI3K inhibitors, it is not clear whether a more selective inhibitor of a particular PI3K isoform may lead to fewer unwanted biological effects. It has recently been reported that non-PI3Kα class I isoforms (PI3Kβ, δ and δ) have the ability to induce oncogenic transformation of cells, suggesting that nonisoform-specific inhibitors may offer enhanced therapeutic potential over specific inhibitors.
Selectivity versus other related kinases is also an important consideration for the development of PI3K inhibitors. While selective inhibitors may be preferred in order to avoid unwanted side effects, there have been reports that inhibition of multiple targets in the PI3K/Akt pathway (e.g., PI3Kα and mTOR [mammalian target of rapamycin]) may lead to greater efficacy. It is possible that lipid kinase inhibitors may parallel protein kinase inhibitors in that nonselective inhibitors may also be brought forward to the clinic.

Mammalian Target of Rapamycin, mTOR, is a cell-signaling protein that regulates the response of tumor cells to nutrients and growth factors, as well as controlling tumor blood supply through effects on Vascular Endothelial Growth Factor, VEGF. Inhibitors of mTOR starve cancer cells and shrink tumors by inhibiting the effect of mTOR. All mTOR inhibitors bind to the mTOR kinase. This has at least two important effects. First, mTOR is a downstream mediator of the PI3K/Akt pathway. The PI3K/Akt pathway is thought to be over-activated in numerous cancers and may account for the widespread response from various cancers to mTOR inhibitors. The over-activation of the upstream pathway would normally cause mTOR kinase to be over-activated as well. However, in the presence of mTOR inhibitors, this process is blocked. The blocking effect prevents mTOR from signaling to downstream pathways that control cell growth. Over-activation of the PI3K/Akt kinase pathway is frequently associated with mutations in the PTEN gene, which is common in many cancers and may help predict what tumors will respond to mTOR inhibitors. The second major effect of mTOR inhibition is anti-angiogenesis, via the lowering of VEGF levels.

In lab tests, certain chemotherapy agents were found to be more effective in the presence of mTOR inhibitors. George, J. N., et al., Cancer Research, 61, 1527-1532, 2001. Additional lab results have shown that some rhabdomyosarcoma cells die in the presence of mTOR inhibitors. The complete functions of the mTOR kinase and the effects of mTOR inhibition are not completely understood.

There are three mTOR inhibitors, which have progressed into clinical trials. These compounds are Wyeth's Torisel, also known as 42-(3-hydroxy-2-(hydroxymethyl)-rapamycin 2-methylpropanoate, CCI-779 or Temsirolimus; Novartis' Everolimus, also known as 42-O-(2-hydroxyethyl)-rapamycin, or RAD 001; and Ariad's AP23573 also known as 42-(dimethylphopsinoyl)-rapamycin. The FDA has approved Torisel for the treatment of advanced renal cell carcinoma. In addition, Torisel is active in a NOS/SCID xenograft mouse model of acute lymphoblastic leukemia [Teachey et al, Blood, 107(3), 1149-1155, 2006]. On Mar. 30, 2009, the Food and Drug Administration (FDA) approved Everolimus (AFINITOR™) for the treatment of patients with advanced renal cell carcinoma. AP23573 has been given orphan drug and fast-track status by the FDA for treatment of soft-tissue and bone sarcomas.

The three mTOR inhibitors have non-linear, although reproducible pharmacokinetic profiles. Mean area under the curve (AUC) values for these drugs increase at a less than dose related way. The three compounds are all semi-synthetic derivatives of the natural macrolide antibiotic rapamycin. It would be desirable to find fully synthetic compounds, which inhibit mTOR that are more potent and exhibit improved pharmacokinetic behaviors.

As explained above, PI3K inhibitors and mTOR inhibitors are expected to be novel types of medicaments useful against cell proliferation disorders, especially as carcinostatic agents. Thus, it would be advantageous to have new PI3K inhibitors and mTOR inhibitors as potential treatment regimens for mTOR- and PI3K-related diseases. The instant invention is directed to these and other important ends.

### SUMMARY OF THE INVENTION

In one aspect, the invention provides compounds of the Formula I:

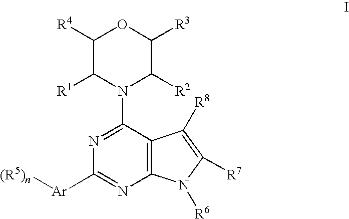

or a pharmaceutically acceptable salt thereof, wherein the constituent variables are as defined below.

### DETAILED DESCRIPTION OF THE INVENTION

In one aspect, the invention provides compounds of the Formula I:

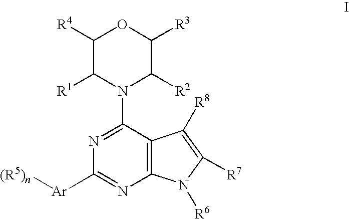

or a pharmaceutically acceptable salt thereof wherein;
R¹, R², R³, and R⁴ are each independently H or C₁-C₆alkyl-;
   or either R¹ and R² or R³ and R⁴ together may form an C₁-C₃alkylene chain which, when taken together with the morpholine ring to which said chain is attached, forms a bridged, bicyclic ring, and optionally one CH₂ group in the C₁-C₃alkylene chain is replaced with —N(H)—, —N(C₁-C₆alkyl)-, —N(C₆-C₁₄aryl)-, —S—, —SO—, —S(O)₂—, or —O—;
Ar is phenyl, naphthyl, or a nitrogen-containing mono- or bicyclic heteroaryl-;
n is 0, 1, 2, or 3;
R⁵ is independently:
a) C₁-C₈acyl-,
b) C₁-C₆alkyl-, which is optionally substituted with from 1 to 3 substituents independently selected from:
   i) H₂N—,
   ii) (C₁-C₆alkyl)amino-,
   iii) di(C₁-C₆alkyl)amino-, and
   iv) C₁-C₉heterocyclyl-,
c) (C₁-C₆alkyl)amido-,
d) (C₁-C₆alkyl)carboxyl-,
e) (C₁-C₆alkyl)carbonylamido-,
f) C₁-C₆alkoxy- optionally substituted by C₁-C₆alkoxy- or C₁-C_{g}heteroaryl-,
g) (C₁-C₆alkoxy)carbonyl-,
h) (C₆-C₁₄aryl)oxy-,
i) C₃-C₈cycloalkyl-,
j) halo-,
k) C₁-C₆haloalkyl-,
l) C₁-C₉heterocyclyl- optionally substituted by C₁-C₆alkyl- or C₁-C₆hydroxylalkyl-,
m) heterocyclyl(C₁-C₆alkyl)- optionally substituted by C₁-C₆alkyl-,
n) hydroxyl-,
o) C₁-C₆hydroxylalkyl-,
p) C₁-C₆perfluoroalkyl-,
q) C₁-C₆perfluoroalkyl-O—,
r) R⁹R¹⁰N—,
s) C₁-C₉heterocyclyl-,
t) —CN,
u) HO₂C—,
v) R⁹R¹⁰NC(O)—,
w) C₁-C₉heterocyclyl-C(O)—,
x) R⁹C(O)NH—,
y) R⁹R¹⁰NS(O)₂—,
z) R⁹R¹⁰NC(O)NHC(O)NH—,
aa) R¹¹OC(O)NHC(O)NH—,
bb) C₁-C₆alkoxy-C₁-C₆alkylene-NH—C₁-C₆alkylene-,
cc) C₁-C₆hydroxylalkyl-NH—C₁-C₆alkylene-,
dd) amino(C₁-C₆alkyl)-NH—C₁-C₆alkylene-,
ee) di(C₁-C₆alkyl)amino-C₁-C₆alkylene-NH—C₁-C₆alkylene-,
ff) C₁-C₆hydroxylalkyl-NH—,
gg) amino(C₁-C₆alkyl)-NH—,
hh) (C₁-C₆alkyl)N-alkylamido-,
ii) R⁹R¹⁰NC(O)NH—,
jj) C₁-C₉heterocyclyl-C(O)NH—,
kk) R¹¹OC(O)NH—,
ll) R¹¹S(O)₂NH—,
mm) R¹¹S(O)₂—,
nn) —C(═N—(OR⁹))—(NR⁹R¹⁰), or
oo) O₂N—;
   R⁹ and R¹⁰ are each independently H; C₁-C₆alkyl- optionally substituted with from 1 to 3 substituents independently selected from C₁-C₆alkoxy-, H₂N—, (C₁-C₆alkyl)amino-, di(C₁-C₆alkyl)amino-, C₆-C₁₄aryl-, C₁-C₉heterocyclyl- optionally substituted by C₁-C₆alkyl-, and C₁-C₉heteroaryl-; C₁-C₆alkoxy-; C₁-C₉heteroaryl- optionally substituted with from 1 to 3 substituents independently selected from C₁-C₆alkyl- optionally substituted with H₂N—, (C₁-C₆alkyl)amino-, or di(C₁-C₆alkyl)amino-, heterocyclyl(C₁-C₆alkyl)-, halogen, hydroxyl, H₂N—, O₂N—, H₂NSO₂—, HO₂C—, (C₁-C₆alkoxy)carbonyl-, (C₁-C₆alkoxy)C(O)NH—, (C₁-C₆alkyl)amino-, di(C₁-C₆alkyl)amino-, R¹⁶R¹⁷NC(O)—, R¹⁶O—, R¹⁶R¹⁷N—, R¹⁶R¹⁷NS(O)₂—, R¹⁶S(O)₂NR¹⁷—, R¹⁶R¹⁷NC(O)NH—, R¹⁶S—, R¹⁶S(O)—, R¹⁶S(O)₂—, R¹⁶C(O)—, C₁-C₉heterocyclyl- optionally substituted by C₁-C₆alkyl- or C₁-C₆hydroxylalkyl-, C₁-C₆hydroxylalkyl-, and perfluoro(C₁-C₆)alkyl-; C₁-C₆hydroxylalkyl-; C₁-C₉heterocyclyl-; C₆-C₁₄aryl- optionally substituted with from 1 to 3 substituents independently selected from C₁-C₆alkyl- optionally substituted with H₂N—, (C₁-C₆alkyl)amino-, or di(C₁-C₆alkyl)amino-, heterocyclyl(C₁-C₆alkyl)-, halogen, hydroxyl, H₂N—, O₂N—, H₂NSO₂—, HO₂C—, (C₁-C₆alkoxy)carbonyl-, (C₁-C₆alkoxy)C(O)NH—, (C₁-C₆alkyl)amino-, di(C₁-C₆alkyl)amino-, R¹⁶R¹⁷NC(O)—, R¹⁶O—, R¹⁶R¹⁷N—, R¹⁶R¹⁷NS(O)₂—, R¹⁶S(O)₂NR¹⁷—, R¹⁶R¹⁷NC(O)NH—, R¹⁶S—, R¹⁶S(O)—, R¹⁶S(O)₂—, R¹⁶C(O)—, C₁-C₉heterocyclyl- optionally substituted by C₁-C₆alkyl- or C₁-C₆hydroxylalkyl-, C₁-C₆hydroxylalkyl-, and perfluoro(C₁-C₆)alkyl-; or C₃-C₈cycloalkyl-;
   or R⁹ and R¹⁰, when taken together with the nitrogen to which they are attached, form a 3- to 7-membered heterocycle wherein up to two of the carbon atoms of the heterocycle are optionally replaced with —N(H)—, —N(C₁-C₆alkyl)-, —N(C₆-C₁₄aryl)-, —S—, —SO—, —S(O)₂—, or —O—;
   R¹¹ is C₁-C₆alkyl-; C₆-C₁₄aryl-; (C₆-C₁₄aryl)alkyl-, optionally substituted by NH₂; C₁-C₉heterocyclyl-; C₃-C₈cycloalkyl-; C₁-C₆hydroxylalkyl-; or C₁-C₆perfluoroalkyl-;
   R¹⁶ and R¹⁷ are each independently H; C₁-C₆alkyl-; C₁-C₆alkoxy(C₂-C₆alkylene)-; (C₁-C₆alkyl)amino-C₂-C₆alkylene-; di(C₁-C₆alkyl)amino-C₂-C₆alkylene-; C₂-C₆alkenyl; C₂-C₆alkynyl; C₆-C₁₄aryl-; (C₆-C₁₄aryl)alkyl-; C₃-C₈cycloalkyl-; C₁-C₉heteroaryl- optionally substituted by CH₃NHC(O)—; (C₁-C₉heteroaryl)alkyl-; C₁-C₉heterocyclyl-; or heterocyclyl(C₁-C₆alkyl);
   or R¹⁶ and R¹⁷, when taken together with the nitrogen to which they are attached, form a 3- to 7-membered heterocycle wherein up to two of the carbon atoms of the heterocycle are optionally replaced with —N(H)—, —N(C₁-C₆alkyl)-, —N(C₃-C₈cycloalkyl)-, —N(C₆-C₁₄aryl)-, —N(C₁-C₉heteroaryl)-, —S—, —SO—, —S(O)₂—, or —O— and wherein any carbon atom of the heterocycle is optionally substituted with from 1 or 2 substituents independently selected from C₁-C₆alkyl-, H₂N—, (C₁-C₆alkyl)amino-, di(C₁-C₆alkyl)amino-, and C₁-C₉heterocyclyl-;
R⁶ is:
a) hydrogen;
b) C₁-C₆alkyl- optionally substituted with from 1 to 3 substituents independently selected from:
   i) C₁-C₆alkoxy-,
   ii) (C₁-C₆alkyl)amino-,
   iii) di(C₁-C₆alkyl)amino-,
   iv) —CHO,
   v) HO₂C—, and
   vi) (C₁-C₆alkoxy)carbonyl-;
c) C₁-C₆aminoalkyl- optionally substituted with a substituent selected from:
   i) C₆-C₁₄aryl- optionally substituted with halogen,
   ii) (C₁-C₉heteroaryl)alkyl-,
   iii) (C₆-C₁₄aryl)alkyl
   iv) H₂N—C₁-C₆alkylene-,
   v) (C₁-C₆alkyl)amino-C₁-C₆alkylene-, or
   vi) di(C₁-C₆alkyl)amino-C₁-C₆alkylene-;
d) carbonylamidoalkyl- optionally substituted with a substituent selected from:
   i) halogen, or
   ii) di(C₁-C₆alkyl)amino-;
e) C₃-C₈cycloalkyl-;
f) C₆-C₁₄aryl- optionally substituted with a substituent selected from:
   i) HO₂C—,
   ii) C₁-C₆hydroxylalkyl-,
   iii) R¹²R¹³NC(O)—, or
   iv) (C₁-C₆alkoxy)carbonyl-;
g) C₁-C₉heterocycle optionally substituted with from 1 to 3 substituents independently selected from:
   i) C₁-C₈acyl, wherein the C₁-C₈acyl is optionally substituted with a NH₂,
   ii) C₁-C₆alkyl-,
   iii) (C₁-C₉heteroaryl)alkyl- wherein the ring portion of the (C₁-C₉heteroaryl)alkyl- group is optionally substituted with from 1 to 3 substituents independently selected from:
      A) C₁-C₆alkylC(O)NH—,
      B) halogen,
      C) NH₂, and
      D) C₁-C₆alkyl-,
   iv) heterocyclyl(C₁-C₆alkyl)-, wherein the ring portion of the heterocyclyl(C₁-C₆alkyl) group is optionally substituted by a (C₆-C₁₄aryl)alkyl-,
   v) (C₆-C₁₄aryl)alkyl-, wherein the ring portion of the (C₆-C₁₄aryl)alkyl- group is optionally substituted by 1 to 3 substituents independently selected from:
      A) halogen,
      B) C₁-C₆alkyl-,
      C) di(C₁-C₆alkyl)amino-(C₁-C₆alkylene)-O—, and
      D) C₁-C₉heteroaryl-; and
   vi) (C₁-C₆alkoxy)carbonyl-;
h) heterocyclyl(C₁-C₆alkyl) optionally substituted with a substituent selected from:
   i) C₁-C₆alkyl-,
   ii) C₃-C₈cycloalkyl-,
   iii) (C₁-C₆alkoxy)carbonyl-,
   iv) C₁-C₆alkylcarboxy,
   v) (C₆-C₁₄aryl)alkyl- wherein the ring portion of the (C₆-C₁₄aryl)alkyl- group is optionally substituted with a substituent selected from:
      A) halogen,
      B) C₁-C₉heteroaryl-, or
      C) di(C₁-C₆alkyl)amino-(C₁-C₆alkylene)-O—,
   vi) (C₁-C₉heteroaryl)alkyl- wherein the ring portion of the (C₁-C₉heteroaryl)alkyl- group is optionally substituted by a halogen, or
   vii) C₁-C₈acyl, wherein the C₁-C₈acyl is optionally substituted with from 1 to 3 independently selected halogens,
i) (C₁-C₉heteroaryl)alkyl- wherein the ring portion of the (C₁-C₉heteroaryl)alkyl- is optionally substituted by 1 to 3 substituents independently selected from:
   i) R¹²R¹³NC(O)NH—,
   ii) (C₁-C₆alkoxy)carbonyl-,
   iii) HO₂C—,
   iv) hydroxyl, and
   v) R¹²R¹³NC(O);
j) (C₆-C₁₄aryl)alkyl- wherein the ring portion of the (C₆-C₁₄aryl)alkyl- group is optionally by 1 to 3 substituents independently selected from:
   i) R¹²R¹³NC(O)NH—,
   ii) (C₁-C₆alkoxy)carbonyl-,
   iii) HO₂C—,
   iv) hydroxyl, and
   v) R¹²R¹³NC(O);
k) C₁-C₆hydroxylalkyl-;
i) C₁-C₆perfluoroalkyl-; or
m) C₁-C₉heteroaryl- optionally substituted with a substituent selected from:
   i) HO₂C—,
   ii) C₁-C₆hydroxylalkyl-,
   iii) R¹²R¹³NC(O)—, or
   iv) (C₁-C₆alkoxy)carbonyl-;
R¹² and R¹³ are each independently:
a) H;
b) C₁-C₆alkyl- optionally substituted with a substituent selected from:
   i) C₁-C₆alkylC(O)NH—,
   ii) H₂N—,
   iii) (C₁-C₆alkyl)amino-, or
   iv) di(C₁-C₆alkyl)amino-,
c) C₃-C₈cycloalkyl-;
d) C₆-C₁₄aryl- optionally substituted with a substituent selected from:
   i) halogen, or
   ii) monocyclic C₁-C₆heterocycle wherein the monocyclic C₁-C₆heterocycle is optionally substituted with (C₁-C₆alkoxy)carbonyl-;
e) C₁-C₉heteroaryl-;
f) (C₁-C₉heteroaryl)alkyl-;
g) heterocyclyl(C₁-C₆alkyl)-;
h) (C₆-C₁₄aryl)alkyl-, wherein the chain portion of the (C₆-C₁₄aryl)alkyl- group is optionally substituted by a hydroxyl; or
i) monocyclic C₁-C₆heterocyclyl- optionally substituted with a (C₁-C₆alkoxy)carbonyl-;
   or R¹² and R¹³, when taken together with the nitrogen to which they are attached, form a 3- to 7-membered heterocycle wherein up to two of the carbon atoms of the heterocycle are optionally replaced with —N(H)—, —N(C₁-C₆alkyl)-, —N(C₆-C₁₄aryl)-, —S—, —SO—, —S(O)₂—, or —O—;
   R⁷ and R⁸ are each independently hydrogen; halogen; C₁-C₈acyl-; (C₁-C₆alkoxy)carbonyl-; C₁-C₆alkyl- optionally substituted with from 1 to 3 substituents independently selected from halogen, H₂N—, (C₁-C₆alkyl)amino-, di(C₁-C₆alkyl)amino-, (C₁-C₆alkyl)C(O)N(C₁-C₃alkyl)-, (C₁-C₆alkyl)carbonylamido-, HC(O)NH—, H₂NC(O)—, (C₁-C₆alkyl)NHC(O)—, di(C₁-C₆alkyl)NC(O)—, —CN, hydroxyl, C₁-C₆alkoxy-, HO₂C—, (C₁-C₆alkoxy)carbonyl-, —C(O)C₁-C₆alkyl-, C₆-C₁₄aryl-, C₁-C₉heteroaryl-, and C₃-C₈cycloalkyl-; C₂-C₆alkenyl- optionally substituted with from 1 to 3 substituents independently selected from halogen, H₂N—, —NH(C₁-C₆alkyl), di(C₁-C₆alkyl)amino-, (C₁-C₆alkyl)C(O)N(C₁-C₃alkyl)-, (C₁-C₆alkyl)carbonylamido-, HC(O)NH—, H₂NC(O)—, (C₁-C₆alkyl)NHC(O)—, di(C₁-C₆alkyl)NC(O)—, —CN, hydroxyl, C₁-C₆alkoxy-, HO₂C—, (C₁-C₆alkoxy)carbonyl-, —C(O)C₁-C₆alkyl-, C₆-C₁₄aryl-, C₁-C₉heteroaryl-, and C₃-C₈cycloalkyl-; C₂-C₆alkynyl- optionally substituted with from 1 to 3 substituents independently selected from halogen, H₂N—, —NH(C₁-C₆alkyl), di(C₁-C₆alkyl)amino-, (C₁-C₆alkyl)C(O)N(C₁-C₃alkyl)-, (C₁-C₆alkyl)carbonylamido-, HC(O)NH—, H₂NC(O)—, (C₁-C₆alkyl)NHC(O)—, di(C₁-C₆alkyl)NC(O)—, —CN, hydroxyl, —C₁-C₆alkoxy-, HO₂C—, (C₁-C₆alkoxy)carbonyl-, —C(O)C₁-C₆alkyl-, C₆-C₁₄aryl-, C₁-C₉heteroaryl-, and C₃-C₈cycloalkyl-; C₆-C₁₄aryl- optionally substituted with from 1 to 3 substituents independently selected from C₁-C₆alkyl-, halogen, haloalkyl-, hydroxyl, C₁-C₆hydroxyalkyl-, H₂N—, (C₁-C₆alkyl)amino-, di(C₁-C₆alkyl)amino-, HO₂C—, (C₁-C₆alkoxy)carbonyl-, —OC(O)—(C₁-C₆alkyl), —N—(C₁-C₆)alkylamido, H₂NC(O)—, -alkylcarboxamido and O₂N—; C₁-C₉heteroaryl- optionally substituted with from 1 to 3 substituents independently selected from C₁-C₆alkyl-, halogen, -haloalkyl-, hydroxyl, C₁-C₆hydroxylalkyl-, H₂N—, aminoalkyl-, di(C₁-C₆alkyl)amino-, HO₂C—, (C₁-C₆alkoxy)carbonyl-, —OC(O)—(C₁-C₆alkyl), —N—(C₁-C₆)alkylamido, H₂NC(O)—, -alkylcarboxamido and O₂N—; C₁-C₆perfluoroalkyl-; R¹⁴R¹⁵N; R¹⁴R¹⁵NS(O)₂—; or R¹⁴R¹⁵NC(O)—;
   R¹⁴ and R¹⁵ are each independently H; C₁-C₆alkyl- optionally substituted with from 1 to 3 substituents independently selected from C₁-C₆alkoxy-, H₂N—, (C₁-C₆alkyl)amino-, di(C₁-C₆alkyl)amino-, C₆-C₁₄aryl-, C₁-C₉heterocyclyl-, and C₁-C₉heteroaryl-; C₁-C₆alkoxy-; C₁-C₉heteroaryl-; hydroxyl; C₆-C₁₄aryl- optionally substituted with from 1 to 3 substituents independently selected from C₁-C₆alkyl-, halogen, and perfluoro(C₁-C₆)alkyl-; or C₃-C₈cycloalkyl-;
   or R¹⁴ and R¹⁵, when taken together with the nitrogen to which they are attached, form a 3- to 7-membered heterocycle wherein up to two of the carbon atoms of the heterocycle are optionally replaced with —N(H)—, —N(C₁-C₆alkyl)-, —N(C₆-C₁₄aryl)-, —S—, —SO—, —S(O)₂—, or —O—.

In one embodiment, R¹ is H.

In one embodiment, R² is H.

In one embodiment, R³ is H.

In one embodiment, R⁴ is H.

In one embodiment, Ar is phenyl.

In one embodiment, n is 1.

In one embodiment, R⁵ is R⁹R¹⁰NC(O)NH—.

In one embodiment, R⁹ is C₆-C₁₄aryl- substituted with R¹⁶R¹⁷NC(O)—.

In one embodiment, R¹⁶ is di(C₁-C₆alkyl)amino-C₂-C₆alkylene-.

In one embodiment, R¹⁶ is 2-(dimethylamino)ethyl.

In one embodiment, R¹⁷ is H.

In one embodiment, R¹⁰ is H.

In one embodiment, R⁶ is C₁-C₆perfluoroalkyl-.

In one embodiment, R⁶ is 1,1,1-trifluoroethyl.

In one embodiment, R⁷ is H.

In one embodiment, R⁸ is H.

In one embodiment, R⁹ is C₁-C₉heteroaryl-; C₁-C₆hydroxylalkyl-; or C₆-C₁₄aryl- optionally substituted with from 1 to 3 substituents independently selected from C₁-C₆alkyl-, halogen, C₁-C₆hydroxylalkyl-, and perfluoro(C₁-C₆)alkyl.

In one embodiment, R⁹ is pyridyl.

In one embodiment, R⁹ is 4-pyridyl.

In one embodiment, R⁶ is C₁-C₆alkyl- optionally substituted with from 1 to 3 substituents independently selected from C₁-C₆alkoxy-, H₂N—, (C₁-C₆alkyl)amino-, di(C₁-C₆alkyl)amino-, CHO, HO₂C—, and (C₁-C₆alkoxy)carbonyl-; heterocyclyl(C₁-C₆alkyl); C₁-C₆hydroxylalkyl-; or C₁-C₆perfluoroalkyl-.

In one embodiment, R¹═R²═R³═R⁴═H.

In one embodiment, R¹═R²═R³═R⁴═H and R⁵is R⁹R¹⁰NC(O)NH—.

In one embodiment, R¹═R²═R³═R⁴═R¹⁰═H, R⁵ is R⁹R¹⁰NC(O)NH—, and R⁹ is 4-pyridyl.

In one embodiment, R⁷═R⁸═H.

In one embodiment, R⁶ is C₁-C₆perfluoroalkyl- and R⁷═R⁸═H.

In one embodiment, R⁶ is 1,1,1-trifluoroethyl and R⁷═R⁸═H.

Illustrative compounds of the present invention are set forth below:
[3-(4-morpholin-4-yl-7H-pyrrolo[2,3-d]pyrimidin-2-yl)phenyl]methanol;
3-(4-morpholin-4-yl-7H-pyrrolo[2,3-d]pyrimidin-2-yl)phenol;
2-(1H-indazol-4-yl)-4-morpholin-4-yl-7H-pyrrolo[2,3-d]pyrimidine;
1-[4-(4-morpholin-4-yl-7H-pyrrolo[2,3-d]pyrimidin-2-yl)phenyl]-3-pyridin-4-ylurea;
1-[4-(4-morpholin-4-yl-7H-pyrrolo[2,3-d]pyrimidin-2-yl)phenyl]-3-pyridin-3-ylurea;
3-{7-[2-(dimethylamino)ethyl]-4-morpholin-4-yl-7H-pyrrolo[2,3-d]pyrimidin-2-yl}phenol;
(3-{7-[2-(dimethylamino)ethyl]-4-morpholin-4-yl-7H-pyrrolo[2,3-d]pyrimidin-2-yl}phenyl)methanol;
4-{7-[2-(dimethylamino)ethyl]-4-morpholin-4-yl-7H-pyrrolo[2,3-d]pyrimidin-2-yl}aniline;
1-(4-{7-[2-(dimethylamino)ethyl]-4-morpholin-4-yl-7H-pyrrolo[2,3-d]pyrimidin-2-yl}phenyl)-3-pyridin-3-ylurea;
7-[2-(dimethylamino)ethyl]-4-morpholin-4-yl-N-pyridin-3-yl-2-{4-[(pyridin-3-ylcarbamoyl)amino]phenyl}-7H-pyrrolo[2,3-d]pyrimidine-5-carboxamide;
1-(4-{7-[2-(dimethylamino)ethyl]-4-morpholin-4-yl-7H-pyrrolo[2,3-d]pyrimidin-2-yl}phenyl)-3-pyridin-2-ylurea;
1-(4-{7-[2-(dimethylamino)ethyl]-4-morpholin-4-yl-7H-pyrrolo[2,3-d]pyrimidin-2-yl}phenyl)-3-pyridin-4-ylurea;
1-(4-{7-[2-(dimethylamino)ethyl]-4-morpholin-4-yl-7H-pyrrolo[2,3-d]pyrimidin-2-yl}phenyl)-3-(4-fluorophenyl)urea;
1-[2-(dimethylamino)ethyl]-3-(4-{7-[2-(dimethylamino)ethyl]-4-morpholin-4-yl-7H-pyrrolo[2,3-d]pyrimidin-2-yl}phenyl)urea;
1-(4-{7-[2-(dimethylamino)ethyl]-4-morpholin-4-yl-7H-pyrrolo[2,3-d]pyrimidin-2-yl}phenyl)-3-[3-(dimethylamino)propyl]urea;
1-(4-{7-[2-(dimethylamino)ethyl]-4-morpholin-4-yl-7H-pyrrolo[2,3-d]pyrimidin-2-yl}phenyl)-3-ethylurea;
1-(4-{7-[2-(dimethylamino)ethyl]-4-morpholin-4-yl-7H-pyrrolo[2,3-d]pyrimidin-2-yl}phenyl)-3-methylurea;
1-(4-{7-[2-(dimethylamino)ethyl]-4-morpholin-4-yl-7H-pyrrolo[2,3-d]pyrimidin-2-yl}phenyl)-3-[2-(1H-indol-3-yl)ethyl]urea;
1-[3-({2-[3-(hydroxymethyl)phenyl]-4-morpholin-4-yl-7H-pyrrolo[2,3-d]pyrimidin-7-yl}methyl)phenyl]urea;
1-(4-{7-[3-(carbamoylamino)benzyl]-4-morpholin-4-yl-7H-pyrrolo[2,3-d]pyrimidin-2-yl}phenyl)-3-pyridin-4-ylurea;
1-{4-[7-(2,2-dimethoxyethyl)-4-morpholin-4-yl-7H-pyrrolo[2,3-d]pyrimidin-2-yl]phenyl}-3-pyridin-4-ylurea;
1-{4-[4-morpholin-4-yl-7-(2-oxoethyl)-7H-pyrrolo[2,3-d]pyrimidin-2-yl]phenyl}-3-pyridin-4-ylurea;
1-{4-[4-morpholin-4-yl-7-(2-pyrrolidin-1-ylethyl)-7H-pyrrolo[2,3-d]pyrimidin-2-yl]phenyl}-3-pyridin-4-ylurea;
1-{4-[4-morpholin-4-yl-7-(2-piperidin-1-ylethyl)-7H-pyrrolo[2,3-d]pyrimidin-2-yl]phenyl}-3-pyridin-4-ylurea;
1-[4-(7-{2-[(4-fluorophenyl)amino]ethyl}-4-morpholin-4-yl-7H-pyrrolo[2,3-d]pyrimidin-2-yl)phenyl]-3-pyridin-4-ylurea;
1-[4-(4-morpholin-4-yl-7-{2-[(pyridin-3-ylmethy)amino]ethyl}-7H-pyrrolo[2,3-d]pyrimidin-2-yl)phenyl]-3-pyridin-4-ylurea;
1-{4-[7-(2-{[2-(dimethylamino)ethyl]amino}ethyl)-4-morpholin-4-yl-7H-pyrrolo[2,3-d]pyrimidin-2-yl]phenyl}-3-pyridin-4-ylurea;
1-(4-{7-[2-(4-methylpiperazin-1-yl)ethyl]-4-morpholin-4-yl-7H-pyrrolo[2,3-d]pyrimidin-2-yl}phenyl)-3-pyridin-4-ylurea;
1-{4-[4-morpholin-4-yl-7-(2-piperazin-1-ylethyl)-7H-pyrrolo[2,3-d]pyrimidin-2-yl]phenyl}-3-pyridin-4-ylurea;
1-{4-[7-(2-{[2-(1H-imidazol-5-yl)ethyl]amino}ethyl)-4-morpholin-4-yl-7H-pyrrolo[2,3-d]pyrimidin-2-yl]phenyl}-3-pyridin-4-ylurea;
1-(4-{7-[2-(tert-butylamino)ethyl]-4-morpholin-4-yl-7H-pyrrolo[2,3-d]pyrimidin-2-yl}phenyl)-3-pyridin-4-ylurea;
1-(4-{7-[2-(isopropylamino)ethyl]-4-morpholin-4-yl-7H-pyrrolo[2,3-d]pyrimidin-2-yl}phenyl)-3-pyridin-4-ylurea;
1-(4-{7-[2-(methylamino)ethyl]-4-morpholin-4-yl-7H-pyrrolo[2,3-d]pyrimidin-2-yl}phenyl)-3-pyridin-4-ylurea;
1-{4-[7-(2-hydroxyethyl)-4-morpholin-4-yl-7H-pyrrolo[2,3-d]pyrimidin-2-yl]phenyl}-3-pyridin-4-ylurea;
1-(4-{7-[(2,5-dioxoimidazolidin-4-yl)methyl]-4-morpholin-4-yl-7H-pyrrolo[2,3-d]pyrimidin-2-yl}phenyl)-3-pyridin-4-ylurea;
1-{4-[4-morpholin-4-yl-7-(2,2,2-trifluoroethyl)-7H-pyrrolo[2,3-d]pyrimidin-2-yl]phenyl}-3-pyridin-4-ylurea;
1-{4-[4-morpholin-4-yl-7-(2,2,2-trifluoroethyl)-7H-pyrrolo[2,3-d]pyrimidin-2-yl]phenyl}-3-pyridin-3-ylurea;
1-(4-fluorophenyl)-3-{4-[4-morpholin-4-yl-7-(2,2,2-trifluoroethyl)-7H-pyrrolo[2,3-d]pyrimidin-2-yl]phenyl}urea;
1-[4-(4-methylpiperazin-1-yl)phenyl]-3-{4-[4-morpholin-4-yl-7-(2,2,2-trifluoroethyl)-7H-pyrrolo[2,3-d]pyrimidin-2-yl]phenyl}urea;
1-[4-(hydroxymethyl)phenyl]-3-{4-[4-morpholin-4-yl-7-(2,2,2-trifluoroethyl)-7H-pyrrolo[2,3-d]pyrimidin-2-yl]phenyl}urea;
1-[2-(dimethylamino)ethyl]-3-{4-[4-morpholin-4-yl-7-(2,2,2-trifluoroethyl)-7H-pyrrolo[2,3-d]pyrimidin-2-yl]phenyl}urea;
1-(2-hydroxyethyl)-3-{4-[4-morpholin-4-yl-7-(2,2,2-trifluoroethyl)-7H-pyrrolo[2,3-d]pyrimidin-2-yl]phenyl}urea;
2-hydroxyethyl{4-[4-morpholin-4-yl-7-(2,2,2-trifluoroethyl)-7H-pyrrolo[2,3-d]pyrimidin-2-yl]phenyl}carbamate;
1-[4-(7-methyl-4-morpholin-4-yl-7H-pyrrolo[2,3-d]pyrimidin-2-yl)phenyl]-3-pyridin-3-ylurea;
5-[4-morpholin-4-yl-7-(2,2,2-trifluoroethyl)-7H-pyrrolo[2,3-d]pyrimidin-2-yl]-1H-benzimidazol-2-amine;
1-{5-[4-morpholin-4-yl-7-(2,2,2-trifluoroethyl)-7H-pyrrolo[2,3-d]pyrimidin-2-yl]-1H-benzimidazol-2-yl}-3-pyridin-3-ylurea;
N-{5-[4-morpholin-4-yl-7-(2,2,2-trifluoroethyl)-7H-pyrrolo[2,3-d]pyrimidin-2-yl]-1H-benzimidazol-2-yl}isonicotinamide;
N-methyl-5-[4-morpholin-4-yl-7-(2,2,2-trifluoroethyl)-7H-pyrrolo[2,3-d]pyrimidin-2-yl]-1H-benzimidazol-2-amine;
ethyl{5-[4-morpholin-4-yl-7-(2,2,2-trifluoroethyl)-7H-pyrrolo[2,3-d]pyrimidin-2-yl]-1H-benzimidazol-2-yl}carbamate;
methyl 4-[({4-[4-morpholin-4-yl-7-(2,2,2-trifluoroethyl)-7H-pyrrolo[2,3-d]pyrimidin-2-yl]phenyl}carbamoyl)amino]benzoate;
N-[2-(dimethylamino)ethyl]-N-methyl-4-[({4-[4-morpholin-4-yl-7-(2,2,2-trifluoroethyl)-7H-pyrrolo[2,3-d]pyrimidin-2-yl]phenyl}carbamoyl)amino]benzamide;
N-[2-(dimethylamino)ethyl]-4-[({4-[4-morpholin-4-yl-7-(2,2,2-trifluoroethyl)-7H-pyrrolo[2,3-d]pyrimidin-2-yl]phenyl}carbamoyl)amino]benzamide;
N-methyl-N-[2-(methylamino)ethyl]-4-[({4-[4-morpholin-4-yl-7-(2,2,2-trifluoroethyl)-7H-pyrrolo[2,3-d]pyrimidin-2-yl]phenyl}carbamoyl)amino]benzamide;
1-{4-[(4-methylpiperazin-1-yl)carbonyl]phenyl}-3-{4-[4-morpholin-4-yl-7-(2,2,2-trifluoroethyl)-7H-pyrrolo[2,3-d]pyrimidin-2-yl]phenyl}urea;
1-{4-[(3,3-dimethylpiperazin-1-yl)carbonyl]phenyl}-3-{4-[4-morpholin-4-yl-7(2,2,2-trifluoroethyl)-7H-pyrrolo[2,3-d]pyrimidin-2-yl]phenyl}urea;
4-[({4-[4-morpholin-4-yl-7-(2,2,2-trifluoroethyl)-7H-pyrrolo[2,3-d]pyrimidin-2-yl]phenyl}carbamoyl)amino]-N-(2-piperidin-1-ylethyl)benzamide;
1-(4-{[4-(dimethylamino)piperidin-1-yl]carbonyl}phenyl)-3-{4-[4-morpholin-4-yl-7-(2,2,2-trifluoroethyl)-7H-pyrrolo[2,3-d]pyrimidin-2-yl]phenyl}urea;
1-{4-[2-(dimethylamino)ethoxy]phenyl}-3-{4-[4-morpholin-4-yl-7-(2,2,2-trifluoroethyl)-7H-pyrrolo[2,3-d]pyrimidin-2-yl]phenyl}urea;
methyl 4-({[4-(7-ethyl-4-morpholin-4-yl-7H-pyrrolo[2,3-d]pyrimidin-2-yl)phenyl]carbamoyl}amino)benzoate;
4-({[4-(7-ethyl-4-morpholin-4-yl-7H-pyrrolo[2,3-d]pyrimidin-2-yl)phenyl]carbamoyl}amino)benzoic acid;
N-[2-(dimethylamino)ethyl]-4-({[4-(7-ethyl-4-morpholin-4-yl-7H-pyrrolo[2,3-d]pyrimidin-2-yl)phenyl]carbamoyl}amino)-N-methylbenzamide;
N-[2-(dimethylamino)ethyl]-4-({[4-(7-ethyl-4-morpholin-4-yl-7H-pyrrolo[2,3-d]pyrimidin-2-yl)phenyl]carbamoyl}amino)benzamide;
1-[4-(7-ethyl-4-morpholin-4-yl-7H-pyrrolo[2,3-d]pyrimidin-2-yl)phenyl]-3-{4-[(4-methylpiperazin-1-yl)carbonyl]phenyl}urea;
1-(4-{[(3R,5S)-3,5-dimethylpiperazin-1-yl]carbonyl}phenyl)-3-[4-(7-ethyl-4-morpholin-4-yl-7H-pyrrolo[2,3-d]pyrimidin-2-yl)phenyl]urea;
1-(4-{[4-(dimethylamino)piperidin-1-yl]carbonyl}phenyl)-3-[4-(7-ethyl-4-morpholin-4-yl-7H-pyrrolo[2,3-d]pyrimidin-2-yl)phenyl]urea;
1-[4-(7-ethyl-4-morpholin-4-yl-7H-pyrrolo[2,3-d]pyrimidin-2-yl)phenyl]-3-[4-(morpholin-4-ylcarbonyl)phenyl]urea;
1-[4-(7-ethyl-4-morpholin-4-yl-7H-pyrrolo[2,3-d]pyrimidin-2-yl)phenyl]-3-[4-(piperazin-1-ylcarbonyl)phenyl]urea;
4-({[4-(7-ethyl-4-morpholin-4-yl-7H-pyrrolo[2,3-d]pyrimidin-2-yl)phenyl]carbamoyl}amino)-N-(2-piperidin-1-ylethyl)benzamide;
1-[4-(7-ethyl-4-morpholin-4-yl-7H-pyrrolo[2,3-d]pyrimidin-2-yl)phenyl]-3-{4-[(4-pyrrolidin-1-ylpiperidin-1-yl)carbonyl]phenyl}urea;
1-[4-(7-ethyl-4-morpholin-4-yl-7H-pyrrolo[2,3-d]pyrimidin-2-yl)phenyl]-3-{4-[(4-ethylpiperazin-1-yl)carbonyl]phenyl}urea;
1-[4-(7-ethyl-4-morpholin-4-yl-7H-pyrrolo[2,3-d]pyrimidin-2-yl)phenyl]-3-[4-(thiomorpholin-4-ylcarbonyl)phenyl]urea;
1-[4-(1,4′-bipiperidin-1′-ylcarbonyl)phenyl]-3-[4-(7-ethyl-4-morpholin-4-yl-7H-pyrrolo[2,3-d]pyrimidin-2-yl)phenyl]urea;
1-{4-[(4-cyclopentylpiperazin-1-yl)carbonyl]phenyl}-3-[4-(7-ethyl-4-morpholin-4-yl-7H-pyrrolo[2,3-d]pyrimidin-2-yl)phenyl]urea;
N-[3-(dimethylamino)propyl]-4-({[4-(7-ethyl-4-morpholin-4-yl-7H-pyrrolo[2,3-d]pyrimidin-2-yl)phenyl]carbamoyl}amino)benzamide;
1-[4-(7-ethyl-4-morpholin-4-yl-7H-pyrrolo[2,3-d]pyrimidin-2-yl)phenyl]-3-{4-[(4-pyridin-2-ylpiperazin-1-yl)carbonyl]phenyl}urea;
4-({[4-(7-ethyl-4-morpholin-4-yl-7H-pyrrolo[2,3-d]pyrimidin-2-yl)phenyl]carbamoyl}amino)-N-(2-pyrrolidin-1-ylethyl)benzamide;
1-[4-(7-ethyl-4-morpholin-4-yl-7H-pyrrolo[2,3-d]pyrimidin-2-yl)phenyl]-3-{4-[(4-morpholin-4-ylpiperidin-1-yl)carbonyl]phenyl}urea;
4-({[4-(7-ethyl-4-morpholin-4-yl-7H-pyrrolo[2,3-d]pyrimidin-2-yl)phenyl]carbamoyl}amino)-N-(2-methoxyethyl)benzamide;
1-[4-(7-isopropyl-4-morpholin-4-yl-7H-pyrrolo[2,3-d]pyrimidin-2-yl)phenyl]-3-pyridin-4-ylurea;
1-[4-(7-isopropyl-4-morpholin-4-yl-7H-pyrrolo[2,3-d]pyrimidin-2-yl)phenyl]-3-[4-(4-methylpiperazin-1-yl)phenyl]urea;
1-{4-[2-(dimethylamino)ethoxy]phenyl}-3-[4-(7-isopropyl-4-morpholin-4-yl-7H-pyrrolo[2,3-d]pyrimidin-2-yl)phenyl]urea;
1-[4-(7-isopropyl-4-morpholin-4-yl-7H-pyrrolo[2,3-d]pyrimidin-2-yl)phenyl]-3-{4-[(4-methylpiperazin-1-yl)carbonyl]phenyl}urea;
1-[4-(7-isopropyl-4-morpholin-4-yl-7H-pyrrolo[2,3-d]pyrimidin-2-yl)phenyl]-3-[4-(piperazin-1-ylcarbonyl)phenyl]urea;
1-(4-{[4-(dimethylamino)piperidin-1-yl]carbonyl}phenyl)-3-[4-(7-isopropyl-4-morpholin-4-yl-7H-pyrrolo[2,3-d]pyrimidin-2-yl)phenyl]urea;
N-[2-(dimethylamino)ethyl]-4-({[4-(7-isopropyl-4-morpholin-4-yl-7H-pyrrolo[2,3-d]pyrimidin-2-yl)phenyl]carbamoyl}amino)-N-methylbenzamide;
N-[2-(dimethylamino)ethyl]-4-({[4-(7-isopropyl-4-morpholin-4-yl-7H-pyrrolo[2,3-d]pyrimidin-2-yl)phenyl]carbamoyl}amino)benzamide;
4-({[4-(7-isopropyl-4-morpholin-4-yl-7H-pyrrolo[2,3-d]pyrimidin-2-yl)phenyl]carbamoyl}amino)-N-(2-pyrrolidin-1-ylethyl)benzamide;
1-[4-(7-isopropyl-4-morpholin-4-yl-7H-pyrrolo[2,3-d]pyrimidin-2-yl)phenyl]-3-{4-[(4-pyrrolidin-1-ylpiperidin-1-yl)carbonyl]phenyl}urea;
methyl 4-({[4-(7-isopropyl-4-morpholin-4-yl-7H-pyrrolo[2,3-d]pyrimidin-2-yl)phenyl]carbamoyl}amino)benzoate;
4-({[4-(7-isopropyl-4-morpholin-4-yl-7H-pyrrolo[2,3-d]pyrimidin-2-yl)phenyl]carbamoyl}amino)benzoic acid;
1-[4-(7-ethyl-4-morpholin-4-yl-7H-pyrrolo[2,3-d]pyrimidin-2-yl)phenyl]-3-(4-{[4-(1-methylethyl)piperazin-1-yl]carbonyl}phenyl)urea;
1-{4-[(4-ethylpiperazin-1-yl)carbonyl]phenyl}-3-{4-[7-(1-methylethyl)-4-morpholin-4yl-7H-pyrrolo[2,3-d]pyrimidin-2-yl]phenyl}urea;
1-{4-[7-(1-methylethyl)-4-morpholin-4-yl-7H-pyrrolo[2,3-d]pyrimidin-2-yl]phenyl}-3-(4-{[4-(1-methylethyl)piperazin-1-yl]carbonyl}phenyl)urea;
tert-butyl 4-(4-morpholin-4-yl-2-{4-[(pyridin-3-ylcarbamoyl)amino]phenyl}-7H-pyrrolo[2,3-d]pyrimidin-7-yl)piperidine-1-carboxylate;
4-[4-morpholin-4-yl-7-(2,2,2-trifluoroethyl)-7H-pyrrolo[2,3-d]pyrimidin-2-yl]aniline;
1-[4-(7-ethyl-4-morpholin-4-yl-7H-pyrrolo[2,3-d]pyrimidin-2-yl)phenyl]-3-methylurea; and
1-(4-{5-[(dimethylamino)methyl]-7-ethyl-4-morpholin-4-yl-7H-pyrrolo[2,3-d]pyrimidin-2-yl}phenyl)-3-methylurea.

Other illustrative compounds of the present invention are set forth below:
1-(4-(4-cyclopropylpiperazine-1-carbonyl)phenyl)-3-(4-(7-isopropyl-4-morpholino-7H-pyrrolo[2,3-d]pyrimidin-2-yl)phenyl)urea;
1-(4-(4-cyclopropylpiperazine-1-carbonyl)phenyl)-3-(4-(7-ethyl-4-morpholino-7H-pyrrolo[2,3-d]pyrimidin-2-yl)phenyl)urea;
1-(4-(4-cyclopropylpiperazine-1-carbonyl)phenyl)-3-(4-(4-morpholino-7-(2,2,2-trifluoroethyl)-7H-pyrrolo[2,3-d]pyrimidin-2-yl)phenyl)urea;
1-(4-(4-cyclopropylpiperazine-1-carbonyl)phenyl)-3-(4-(7-(2-(dimethylamino)ethyl)-4-morpholino-7H-pyrrolo[2,3-d]pyrimidin-2-yl)phenyl)urea;
(S)-1-(4-(3,4-dimethylpiperazine-1-carbonyl)phenyl)-3-(4-(7-isopropyl-4-morpholino-7H-pyrrolo[2,3-d]pyrimidin-2-yl)phenyl)urea;
(S)-1-(4-(3,4-dimethylpiperazine-1-carbonyl)phenyl)-3-(4-(7-ethyl-4-morpholino-7H-pyrrolo[2,3-d]pyrimidin-2-yl)phenyl)urea;
(S)-1-(4-(3,4-dimethylpiperazine-1-carbonyl)phenyl)-3-(4-(4-morpholino-7-(2,2,2-trifluoroethyl)-7H-pyrrolo[2,3-d]pyrimidin-2-yl)phenyl)urea;
(S)-1-(4-(7-(2-(dimethylamino)ethyl)-4-morpholino-7H-pyrrolo[2,3-d]pyrimidin-2-yl)phenyl)-3-(4-(3,4-dimethylpiperazine-1-carbonyl)phenyl)urea;
(R)-1-(4-(3,4-dimethylpiperazine-1-carbonyl)phenyl)-3-(4-(7-isopropyl-4-morpholino-7H-pyrrolo[2,3-d]pyrimidin-2-yl)phenyl)urea;
(R)-1-(4-(3,4-dimethylpiperazine-1-carbonyl)phenyl)-3-(4-(7-ethyl-4-morpholino-7H-pyrrolo[2,3-d]pyrimidin-2-yl)phenyl)urea;
(R)-1-(4-(3,4-dimethylpiperazine-1-carbonyl)phenyl)-3-(4-(4-morpholino-7-(2,2,2-trifluoroethyl)-7H-pyrrolo[2,3-d]pyrimidin-2-yl)phenyl)urea;
(R)-1-(4-(7-(2-(dimethylamino)ethyl)-4-morpholino-7H-pyrrolo[2,3-d]pyrimidin-2-yl)phenyl)-3-(4-(3,4-dimethylpiperazine-1-carbonyl)phenyl)urea;
1-(4-(3-(dimethylamino)pyrrolidine-1-carbonyl)phenyl)-3-(4-(7-isopropyl-4-morpholino-7H-pyrrolo[2,3-d]pyrimidin-2-yl)phenyl)urea;
1-(4-(3-(dimethylamino)pyrrolidine-1-carbonyl)phenyl)-3-(4-(7-ethyl-4-morpholino-7H-pyrrolo[2,3-d]pyrimidin-2-yl)phenyl)urea;
1-(4-(3-(dimethylamino)pyrrolidine-1-carbonyl)phenyl)-3-(4-(4-morpholino-7-(2,2,2-trifluoroethyl)-7H-pyrrolo[2,3-d]pyrimidin-2-yl)phenyl)urea;
1-(4-(7-(2-(dimethylamino)ethyl)-4-morpholino-7H-pyrrolo[2,3-d]pyrimidin-2-yl)phenyl)-3-(4-(3-(dimethylamino)pyrrolidine-1-carbonyl)phenyl)urea;
1-(4-(3-(dimethylamino)pyrrolidine-1-carbonyl)phenyl)-3-(4-(7-ethyl-4-morpholino-7H-pyrrolo[2,3-d]pyrimidin-2-yl)-3-fluorophenyl)urea;
1-(4-(7-ethyl-4-morpholino-7H-pyrrolo[2,3-d]pyrimidin-2-yl)-3-fluorophenyl)-3-(4-(piperazine-1-carbonyl)phenyl)urea;
1-(4-(7-ethyl-4-morpholino-7H-pyrrolo[2,3-d]pyrimidin-2-yl)-3-fluorophenyl)-3-(4-(thiomorpholine-4-carbonyl)phenyl)urea;
1-(4-(7-ethyl-4-morpholino-7H-pyrrolo[2,3-d]pyrimidin-2-yl)-3-fluorophenyl)-3-(4-(morpholine-4-carbonyl)phenyl)urea;
1-(4-(7-ethyl-4-morpholino-7H-pyrrolo[2,3-d]pyrimidin-2-yl)-3-fluorophenyl)-3-(4-(4-methylpiperazine-1-carbonyl)phenyl)urea;
1-(4-(7-ethyl-4-morpholino-7H-pyrrolo[2,3-d]pyrimidin-2-yl)-3-fluorophenyl)-3-(4-(4-ethylpiperazine-1-carbonyl)phenyl)urea;
1-(4-(7-ethyl-4-morpholino-7H-pyrrolo[2,3-d]pyrimidin-2-yl)-3-fluorophenyl)-3-(4-(4-isopropylpiperazine-1-carbonyl)phenyl)urea;
1-(4-(3,4-dimethylpiperazine-1-carbonyl)phenyl)-3-(4-(7-ethyl-4-morpholino-7H-pyrrolo[2,3-d]pyrimidin-2-yl)-3-fluorophenyl)urea;
1-(4-(7-ethyl-4-morpholino-7H-pyrrolo[2,3-d]pyrimidin-2-yl)-3-fluorophenyl)-3-(4-(3,3,4-trimethylpiperazine-1-carbonyl)phenyl)urea;
1-(4-(7-ethyl-4-morpholino-7H-pyrrolo[2,3-d]pyrimidin-2-yl)-3-fluorophenyl)-3-(4-(3,4,5-trimethylpiperazine-1-carbonyl)phenyl)urea;
N-(2-(dimethylamino)ethyl)-4-(3-(4-(7-ethyl-4-morpholino-7H-pyrrolo[2,3-d]pyrimidin-2-yl)-3-fluorophenyl)ureido)benzamide;
N-(2-(dimethylamino)ethyl)-4-(3-(4-(7-ethyl-4-morpholino-7H-pyrrolo[2,3-d]pyrimidin-2-yl)-3-fluorophenyl)ureido)-N-methylbenzamide;
1-(4-(7-ethyl-4-morpholino-7H-pyrrolo[2,3-d]pyrimidin-2-yl)phenyl)-3-(4-(pyridin-4-yloxy)phenyl)urea; and
5-(4-(3-(4-(7-ethyl-4-morpholino-7H-pyrrolo[2,3-d]pyrimidin-2-yl)phenyl)ureido)phenoxy)-N-methylpicolinamide.

In other aspects, the invention provides pharmaceutical compositions comprising compounds or pharmaceutically acceptable salts of the compounds of the present Formula I and a pharmaceutically acceptable carrier.

In other aspects, the invention provides that the pharmaceutically acceptable carrier suitable for oral administration and the composition comprises an oral dosage form.

In other aspects, the invention provides a composition comprising a compound of Formula I; a second compound selected from the group consisting of a topoisomerase I inhibitor, a MEK1/2 inhibitor, a HSP90 inhibitor, procarbazine, dacarbazine, gemcitabine, capecitabine, methotrexate, taxol, taxotere, mercaptopurine, thioguanine, hydroxyurea, cytarabine, cyclophosphamide, ifosfamide, nitrosoureas, cisplatin, carboplatin, mitomycin, dacarbazine, procarbizine, etoposide, teniposide, campathecins, bleomycin, doxorubicin, idarubicin, daunorubicin, dactinomycin, plicamycin, mitoxantrone, L-asparaginase, doxorubicin, epirubicin, 5-fluorouracil, docetaxel, paclitaxel, leucovorin, levamisole, irinotecan, estramustine, etoposide, nitrogen mustards, BCNU, carmustine, lomustine, vinblastine, vincristine, vinorelbine, cisplatin, carboplatin, oxaliplatin, imatinib mesylate, Avastin (bevacizumab), hexamethylmelamine, topotecan, tyrosine kinase inhibitors, tyrphostins, herbimycin A, genistein, erbstatin, hydroxyzine, glatiramer acetate, interferon beta-1a, interferon beta-1b, natalizumab, and lavendustin A; and a pharmaceutically acceptable carrier.

In other aspects, the second compound is Avastin.

In other aspects, the invention provides a method of treating a PI3K-related disorder, comprising administering to a mammal in need thereof a compound of Formula I in an amount effective to treat a PI3K-related disorder.

In other aspects, the PI3K-related disorder is selected from restenosis, atherosclerosis, bone disorders, arthritis, diabetic retinopathy, psoriasis, benign prostatic hypertrophy, atherosclerosis, inflammation, angiogenesis, immunological disorders, pancreatitis, kidney disease, and cancer.

In other aspects, the PI3K-related disorder is cancer.

In other aspects, the cancer is selected from the group consisting of leukemia, skin cancer, bladder cancer, breast cancer, uterus cancer, ovary cancer, prostate cancer, lung cancer, colon cancer, pancreas cancer, renal cancer, gastric cancer, and brain cancer.

In other aspects, the invention provides a method of treating an mTOR-related disorder, comprising administering to a mammal in need thereof a compound of Formula I in an amount effective to treat an mTOR-related disorder.

In other aspects, the mTOR-related disorder is selected from restenosis, atherosclerosis, bone disorders, arthritis, diabetic retinopathy, psoriasis, benign prostatic hypertrophy, atherosclerosis, inflammation, angiogenesis, immunological disorders, pancreatitis, kidney disease, and cancer.

In other aspects, the mTOR-related disorder is cancer.

In other aspects, the cancer is selected from the group consisting of leukemia, skin cancer, bladder cancer, breast cancer, uterus cancer, ovary cancer, prostate cancer, lung cancer, colon cancer, pancreas cancer, renal cancer, gastric cancer, and brain cancer.

In other aspects, the invention provides a method of treating a hSMG-1-related disorder, comprising administering to a mammal in need thereof a compound of Formula I in an amount effective to treat a hSMG-1-related disorder.

In other aspects, the hSMG-1-related disorder is selected from restenosis, atherosclerosis, bone disorders, arthritis, diabetic retinopathy, psoriasis, benign prostatic hypertrophy, atherosclerosis, inflammation, angiogenesis, immunological disorders, pancreatitis, kidney disease, and cancer.

In other aspects, the hSMG-1-related disorder is cancer.

In other aspects, the cancer is selected from the group consisting of leukemia, skin cancer, bladder cancer, breast cancer, uterus cancer, ovary cancer, prostate cancer, lung cancer, colon cancer, pancreas cancer, renal cancer, gastric cancer, and brain cancer.

In other aspects, the invention provides a method of treating advanced renal cell carcinoma, comprising administering to a mammal in need thereof a compound of Formula I in an amount effective to treat advanced renal cell carcinoma.

In other aspects, the invention provides a method of treating acute lymphoblastic leukemia, comprising administering to a mammal in need thereof a compound of Formula I in an amount effective to treat acute lymphoblastic leukemia.

In other aspects, the invention provides a method of treating acute malignant melanoma, comprising administering to a mammal in need thereof a compound of Formula I in an amount effective to treat malignant melanoma.

In other aspects, the invention provides a method of treating soft-tissue or bone sarcoma, comprising administering to a mammal in need thereof a compound of Formula I in an amount effective to treat soft-tissue or bone sarcoma.

In other aspects, the invention provides a method of treating a cancer selected from the group consisting of leukemia, skin cancer, bladder cancer, breast cancer, uterus cancer, ovary cancer, prostate cancer, lung cancer, colon cancer, pancreas cancer, renal cancer, gastric cancer, and brain cancer comprising administering to a mammal in need thereof a composition comprising a compound of Formula I; a second compound selected from the group consisting of a topoisomerase I inhibitor, a MEK1/2 inhibitor, a HSP90 inhibitor, procarbazine, dacarbazine, gemcitabine, capecitabine, methotrexate, taxol, taxotere, mercaptopurine, thioguanine, hydroxyurea, cytarabine, cyclophosphamide, ifosfamide, nitrosoureas, cisplatin, carboplatin, mitomycin, dacarbazine, procarbizine, etoposide, teniposide, campathecins, bleomycin, doxorubicin, idarubicin, daunorubicin, dactinomycin, plicamycin, mitoxantrone, L-asparaginase, doxorubicin, epirubicin, 5-fluorouracil, docetaxel, paclitaxel, leucovorin, levamisole, irinotecan, estramustine, etoposide, nitrogen mustards, BCNU, carmustine, lomustine, vinblastine, vincristine, vinorelbine, cisplatin, carboplatin, oxaliplatin, imatinib mesylate, Avastin (bevacizumab), hexamethylmelamine, topotecan, tyrosine kinase inhibitors, tyrphostins, herbimycin A, genistein, erbstatin, hydroxyzine, glatiramer acetate, interferon beta-1a, interferon beta-1b, natalizumab, and lavendustin A; and a pharmaceutically acceptable carrier in an amount effective to treat the cancer.

In other aspects, the invention provides a method of inhibiting mTOR in a subject, comprising administering to a subject in need thereof a compound of Formula I in an amount effective to inhibit mTOR.

In other aspects, the invention provides a method of inhibiting PI3K in a subject, comprising administering to a subject in need thereof a compound of Formula I in an amount effective to inhibit PI3K.

In other aspects, the invention provides a method of inhibiting hSMG-1 in a subject, comprising administering to a subject in need thereof a compound of Formula I in an amount effective to inhibit hSMG-1.

In other aspects, the invention provides a method of inhibiting mTOR, PI3K, and hSMG-1 together in a subject, comprising administering to a subject in need thereof a compound of Formula I in an amount effective to inhibit mTOR, PI3K, and hSMG-1.

In other aspects, the invention provides a method of synthesizing a compound of Formula I comprising reacting a compound of the formula XXIII with either a reagent of the formula Ar(R⁵)ₙB(OH)₂ or a reagent of the formula Ar(R⁵)ₙSnBu₃

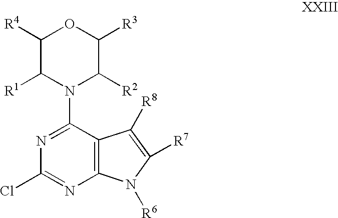

and a suitable catalyst, wherein Ar, n, and R¹-R⁸ are as defined above in formula I, thereby producing a compound of formula I:

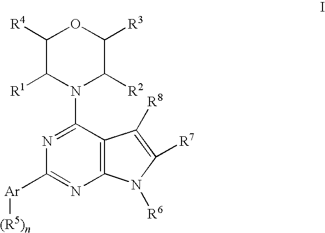

or a pharmaceutically acceptable salt thereof.

In other aspects, the invention provides the method further comprising reacting 2,4-dichloro-7H-pyrrolo[2,3-d]pyrimidine XXI with morpholine or

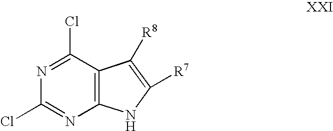

substituted or bridged morpholine V:

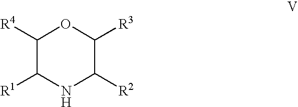

thereby proving mono chloro derivative XXII:

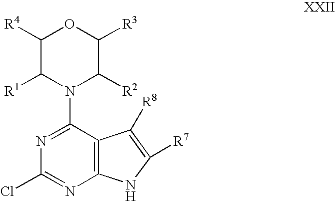

b) optionally alkylating the compound of formula XXII with R⁶X, thereby producing a compound of Formula XXIII, when R⁶ is not H;
   herein R¹-R⁸ are as defined in formula I, except that R⁶ is not H, and wherein X is a leaving group.

Procedures used to synthesize the compounds of the present invention are described in Schemes 1-6 and are illustrated in the examples. Reasonable variations of the described procedures, which would be evident to one skilled in the art, are intended to be within the scope of the present invention:

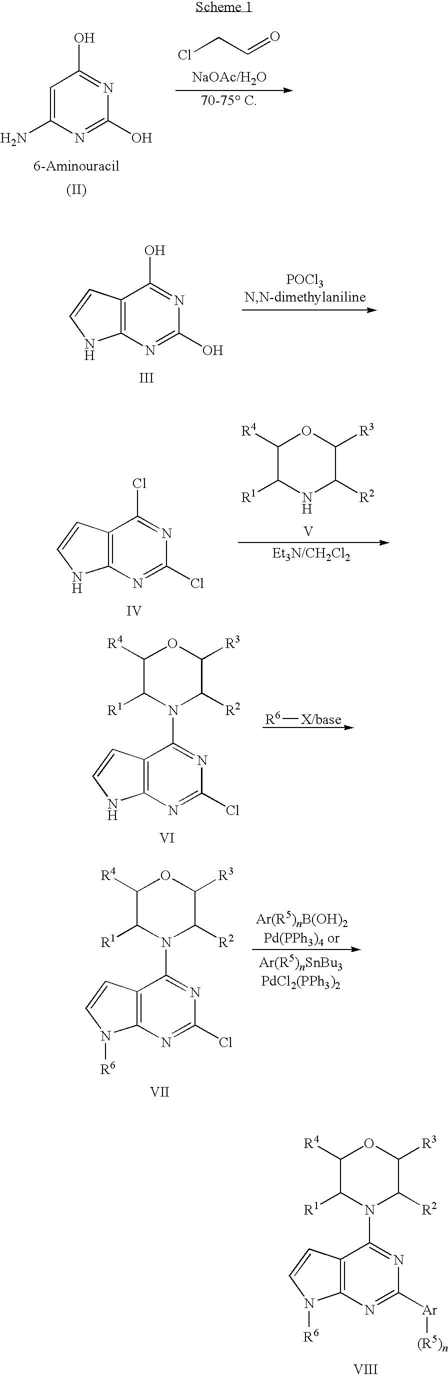

Synthesis of 4-morpholino (substituted or unsubstituted or bridged)-2-aryl (or heteroaryl)-7-substituted (or unsubstituted)-7H-pyrrolo[2,3-d]pyrimidine compounds is shown in Scheme 1. 6-Aminouracil (II) was reacted with the appropriately substituted chloroacetaldehyde derivative to give the core structure III, which was treated with POCl₃ to afford 2,4-dichloro-7H-pyrrolo[2,3-d]pyrimidine IV. Compound IV was reacted with morpholine or substituted or bridged morpholine to provide mono chloro derivative VI. Alkylation of VII gave the intermediate VII, which was converted to the target compound VIII by Suzuki or Stille coupling reaction under the standard thermal conditions or microwave assisted synthesis.

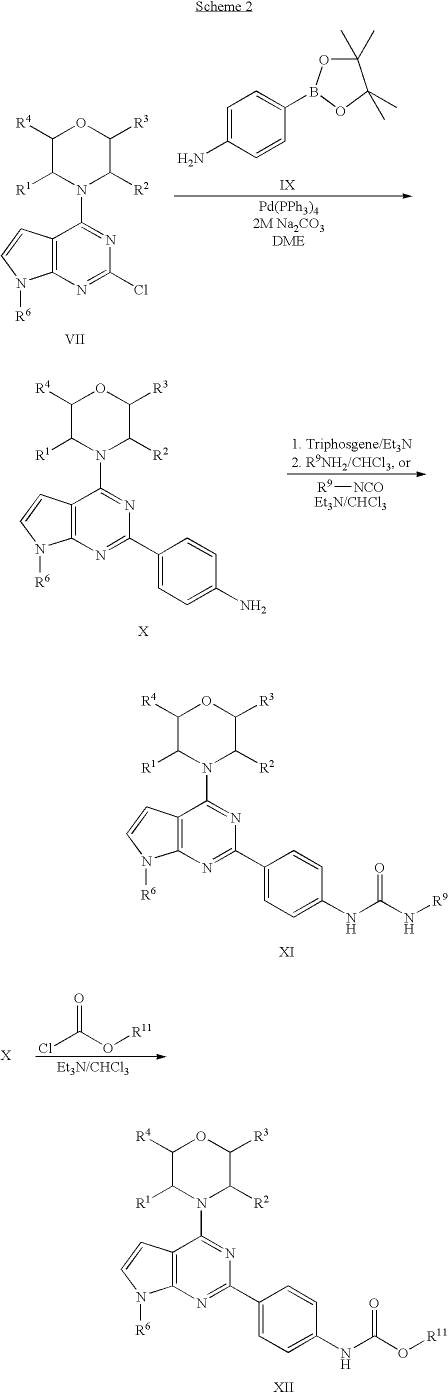

Synthesis of urea analogs of 7H-pyrrolo[2,3-d]pyrimidine compounds XI and XII can be achieved as shown in Scheme 2. Suzuki reaction of intermediate VII (from Scheme 1) with 4-aminophenylboronic acid pinacol ester gave the substituted aniline X, which was reacted different isocyanates or treated with triphosgene followed by different amines to form the urea analog XI. Compound X was treated with alkyl or aryl chloroformate in the presence of triethylamine to give the corresponding carbamate XII.

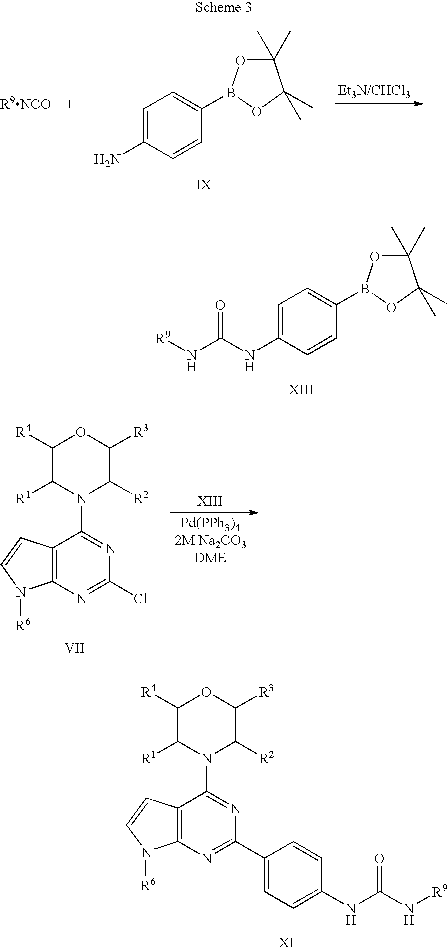

Synthesis of urea analogs of 7H-pyrrolo[2,3-d]pyrimidine compounds XI can be achieved also as shown in Scheme 3. 4-Aminophenylboronic acid pinacol ester IX was reacted with different isocyanates to form 4-ureaphenylboronic esters XIII, which were reacted with VII under the standard Suzuki conditions or microwave assisted conditions to give the target urea analog XI.

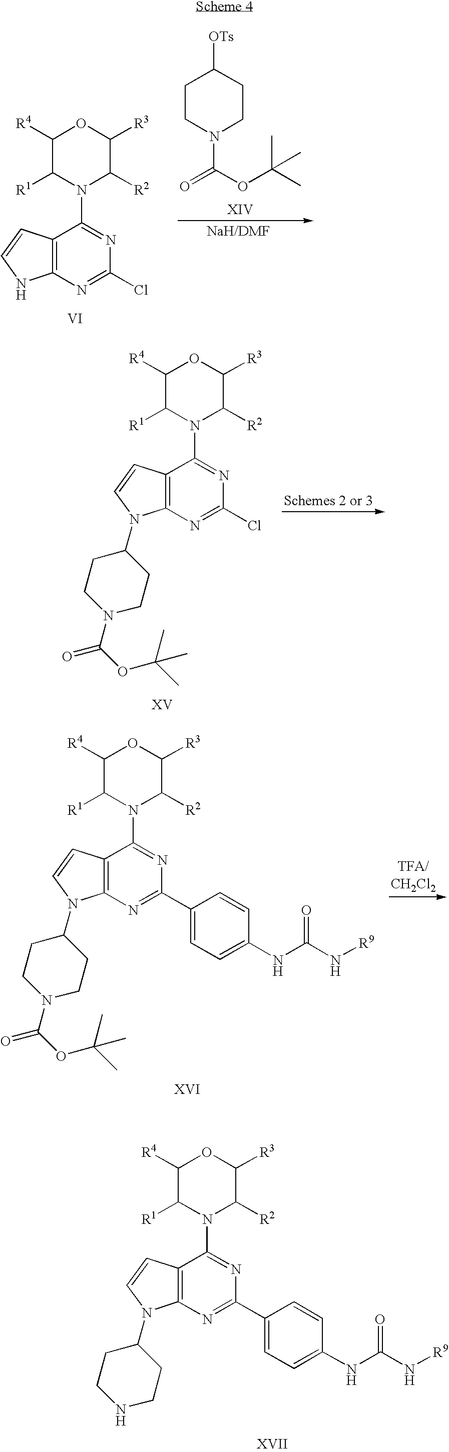

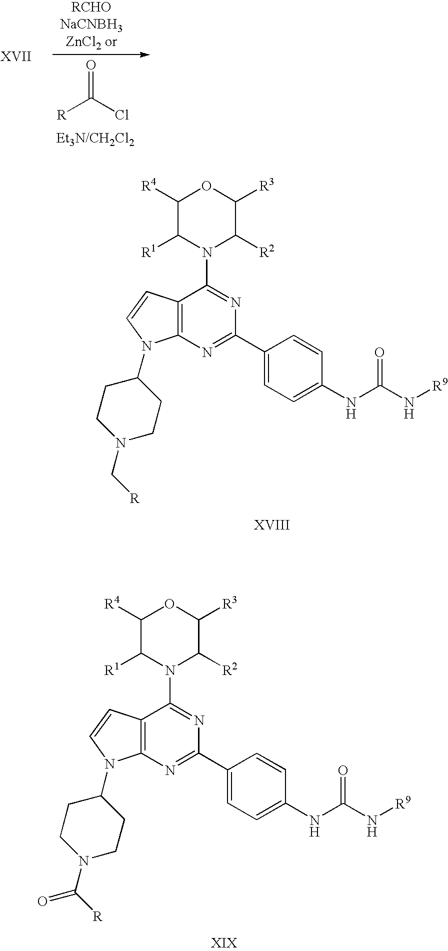

Synthesis of 4-morpholino (substituted or unsubstituted or bridged)-2-aryl (or heteroaryl or urea)-7-substituted piperidin-4-yl-7H-pyrrolo[2,3-d]pyrimidine compounds is shown in Scheme 4. Treatment of intermediate VI (from Scheme 1) with N-BOC protected 4-tosyloxypiperidine (XIV) under basic conditions gave XV, which was converted to the urea analog XVI by the methods shown in Schemes 2 and 3. Deprotection of the BOC group by using TFA provided XVII. Reductive amination of XVII with different aldehydes or ketones in the presence of NaCNBH₃ and ZnCl₂ gave alkylated products XVIII. Alternatively, treatment of XVII with different carboxylic acid chlorides or alkyl/aryl chloroformate in the presence of Et₃N afforded amides or carbamates XIX.

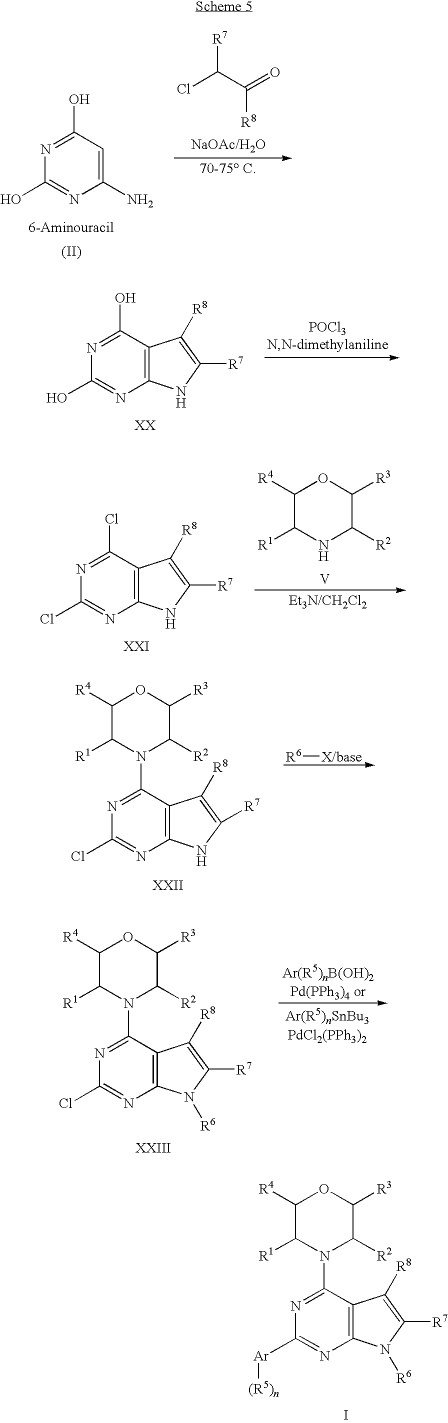

Synthesis of 4-morpholino (substituted or unsubstituted or bridged)-2-aryl (or heteroaryl)-7-substituted (or unsubstituted)-7H-pyrrolo[2,3-d]pyrimidine compounds I is shown in Scheme 5. 6-Aminouracil (II) reacts with the appropriately substituted chloroketone derivative to give the core structure XX, which could be treated with POCl₃ to afford 2,4-dichloro-7H-pyrrolo[2,3-d]pyrimidine XXI. Compound XXI reacts with morpholine or substituted or bridged morpholine V providing mono chloro derivative XXII. Optional alkylation of XXIII gives the intermediate XXIII, which could be converted to the target compound I by Suzuki or Stille coupling reaction under the standard thermal conditions or microwave-assisted synthesis.

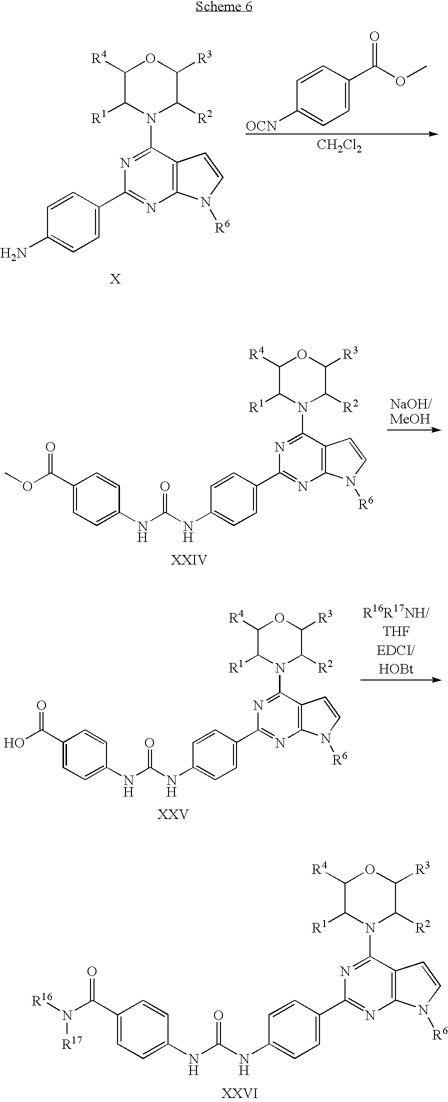

As shown in Scheme 6, reaction of the intermediate aniline X with methyl 4-isocyantobenzoate led to urea ester XXIV, which was converted to the corresponding carboxylic acid XXV by hydrolysis under basic condition. The resulting acid was reacted with different amines catalyzed by EDCI and HOBT to form different amide compounds XXVI.

### Definitions

The following definitions are used in connection with the compounds of the present invention unless the context indicates otherwise. In general, the number of carbon atoms present in a given group is designated “Cₓ-C_{y}”, where x and y are the lower and upper limits, respectively. For example, a group designated as “C₁-C₆” contains from 1 to 6 carbon atoms. The carbon number as used in the definitions herein refers to carbon backbone and carbon branching, but does not include carbon atoms of the substituents, such as alkoxy substitutions and the like. Unless indicated otherwise, the nomenclature of substituents that are not explicitly defined herein are arrived at by naming from left to right the terminal portion of the functionality followed by the adjacent functionality toward the point of attachment. For example, the substituent “arylalkyloxycabonyl” refers to the group (C₆-C₁₄aryl)-(C₁-C₆alkyl)-O—C(O)—. It is understood that the above definitions are not intended to include impermissible substitution patterns (e.g., methyl substituted with 5 fluoro groups). Such impermissible substitution patterns are well known to the skilled artisan.

“Acyl-” refers to a group having a straight, branched, or cyclic configuration or a combination thereof, attached to the parent structure through a carbonyl functionality. Such groups may be saturated or unsaturated, aliphatic or aromatic, and carbocyclic or heterocyclic. Examples of a C₁-C₈acyl- group include HC(O)—, acetyl-, benzoyl-, nicotinoyl-, propionyl-, isobutyryl-, oxalyl-, and the like. Lower-acyl refers to acyl groups containing one to four carbons. An acyl group can be unsubstituted or substituted with one or more of the following groups: halogen, H₂N—, (C₁-C₆alkyl)amino-, di(C₁-C₆alkyl)amino-, (C₁-C₆alkyl)C(O)N(C₁-C₃alkyl)-, (C₁-C₆alkyl)carbonylamido-, HC(O)NH—, H₂NC(O)—, (C₁-C₆alkyl)NHC(O)—, di(C₁-C₆alkyl)NC(O)—, —CN, hydroxyl, C₁-C₆alkoxy-, C₁-C₆alkyl-, HO₂C—, (C₁-C₆alkoxy)carbonyl-, —C(O)(C₁-C₆alkyl), C₆-C₁₄aryl-, C₁-C₉heteroaryl-, or C₃-C₈cycloalkyl-.

“Alkenyl-” refer to a straight or branched chain unsaturated hydrocarbon containing at least one double bond. Examples of a C₂-C₁₀alkenyl- group include, but are not limited to, ethylene, propylene, 1-butylene, 2-butylene, isobutylene, sec-butylene, 1-pentene, 2-pentene, isopentene, 1-hexene, 2-hexene, 3-hexene, isohexene, 1-heptene, 2-heptene, 3-heptene, 1-octene, 2-octene, 3-octene, 4-octene, 1-nonene, 2-nonene, 3-nonene, 4-nonene, 1-decene, 2-decene, 3-decene, 4-decene and 5-decene. An alkenyl-group can be unsubstituted or substituted with one or more of the following groups: halogen, H₂N—, (C₁-C₆alkyl)amino-, di(C₁-C₆alkyl)amino-, (C₁-C₆alkyl)C(O)N(C₁-C₃alkyl)-, (C₁-C₆alkyl)carbonylamido-, HC(O)NH—, H₂NC(O)—, (C₁-C₆alkyl)NHC(O)—, di(C₁-C₆alkyl)NC(O)—, —CN, hydroxyl, C₁-C₆alkoxy-, C₁-C₆alkyl-, HO₂C—, (C₁-C₆alkoxy)carbonyl-, —C(O)(C₁-C₆alkyl), C₆-C₁₄aryl-, C₁-C₉heteroaryl-, and C₃-C₈cycloalkyl-.

“Alkoxy-” refers to the group R—O— where R is an alkyl group, as defined below. Exemplary C₁-C₆alkoxy- groups include but are not limited to methoxy, ethoxy, n-propoxy, 1-propoxy, n-butoxy and t-butoxy. An alkoxy group can be unsubstituted or substituted with one or more of the following groups: halogen, hydroxyl, C₁-C₆alkoxy-, H₂N—, (C₁-C₆alkyl)amino-, di(C₁-C₆alkyl)amino-, (C₁-C₆alkyl)C(O)N(C₁-C₃alkyl)-, (C₁-C₆alkyl)carbonylamido-, HC(O)NH—, H₂NC(O)—, (C₁-C₆alkyl)NHC(O)—, di(C₁-C₆alkyl)NC(O)—, —CN, C₁-C₆alkoxy-, HO₂C—, (C₁-C₆alkoxy)carbonyl-, —C(O)(C₁-C₆alkyl), C₆-C₁₄aryl-, C₁-C₉heteroaryl-, C₃-C₈cycloalkyl-, C₁-C₆haloalkyl-, C₁-C₆aminoalkyl-, (C₁-C₆alkyl)carboxy-, C₁-C₆carbonylamidoalkyl-, or O₂N—.

“(Alkoxy)carbonyl-” refers to the group alkyl-O—C(O)—. Exemplary (C₁-C₆alkoxy)carbonyl- groups include but are not limited to methoxy, ethoxy, n-propoxy, 1-propoxy, n-butoxy and t-butoxy. An (alkoxy)carbonyl group can be unsubstituted or substituted with one or more of the following groups: halogen, hydroxyl, H₂N—, (C₁-C₆alkyl)amino-, di(C₁-C₆alkyl)amino-, (C₁-C₆alkyl)C(O)N(C₁-C₃alkyl)-, (C₁-C₆alkyl)carbonylamido-, HC(O)NH—, H₂NC(O)—, (C₁-C₆alkyl)NHC(O)—, di(C₁-C₆alkyl)NC(O)—, —CN, C₁-C₆alkoxy-, HO₂C—, (C₁-C₆alkoxy)carbonyl-, —C(O)(C₁-C₆alkyl), C₆-C₁₄aryl-, C₁-C₉heteroaryl-, C₃-C₈cycloalkyl-, C₁-C₆haloalkyl-, C₁-C₆aminoalkyl-, (C₁-C₆alkyl)carboxy-, C₁-C₆carbonylamidoalkyl-, or O₂N—.

“Alkyl-” refers to a hydrocarbon chain that may be a straight chain or branched chain, containing the indicated number of carbon atoms, for example, a C₁-C₁₀alkyl- group may have from 1 to 10 (inclusive) carbon atoms in it. In the absence of any numerical designation, “alkyl” is a chain (straight or branched) having 1 to 6 (inclusive) carbon atoms in it. Examples of C₁-C₆alkyl- groups include, but are not limited to, methyl, ethyl, propyl, butyl, pentyl, hexyl, isopropyl, isobutyl, sec-butyl, tert-butyl, isopentyl, neopentyl, and isohexyl. An alkyl- group can be unsubstituted or substituted with one or more of the following groups: halogen, H₂N—, (C₁-C₆alkyl)amino-, di(C₁-C₆alkyl)amino-, (C₁-C₆alkyl)C(O)N(C₁-C₃alkyl)-, (C₁-C₆alkyl)carbonylamido-, HC(O)NH—, H₂NC(O)—, (C₁-C₆alkyl)NHC(O)—, di(C₁-C₆alkyl)NC(O)—, —CN, hydroxyl, C₁-C₆alkoxy-, C₁-C₆alkyl-, HO₂C—, (C₁-C₆alkoxy)carbonyl-, —C(O)(C₁-C₆alkyl), C₆-C₁₄aryl-, C₁-C₉heteroaryl-, C₃-C₈cycloalkyl-, C₁-C₆haloalkyl-, C₁-C₆aminoalkyl-, (C₁-C₆alkyl)carboxy-, C₁-C₆carbonylamidoalkyl-, or O₂N—.

“(Alkyl)amido-” refers to a —C(O)NH— group in which the nitrogen atom of said group is attached to a alkyl group, as defined above. Representative examples of a (C₁-C₆alkyl)amido- group include, but are not limited to, —C(O)NHCH₃, —C(O)NHCH₂CH₃, —C(O)NHCH₂CH₂CH₃, —C(O)NHCH₂CH₂CH₂CH₃, —C(O)NHCH₂CH₂CH₂CH₂CH₃, —C(O)NHCH(CH₃)₂, —C(O)NHCH₂CH(CH₃)₂, —C(O)NHCH(CH₃)CH₂CH₃, —C(O)NH—C(CH₃)₃ and —C(O)NHCH₂C(CH₃)₃.

“(Alkyl)amino-” refers to an —NH group, the nitrogen atom of said group being attached to a alkyl group, as defined above. Representative examples of an (C₁-C₆alkyl)amino- group include, but are not limited to —NHCH₃, —NHCH₂CH₃, —NHCH₂CH₂CH₃, —NHCH₂CH₂CH₂CH₃, —NHCH(CH₃)₂, —NHCH₂CH(CH₃)₂, —NHCH(CH₃)CH₂CH₃ and —NH—C(CH₃)₃. An (alkyl)amino group can be unsubstituted or substituted with one or more of the following groups: halogen, H₂N—, (C₁-C₆alkyl)amino-, di(C₁-C₆alkyl)amino-, (C₁-C₆alkyl)C(O)N(C₁-C₃alkyl)-, (C₁-C₆alkyl)carbonylamido-, HC(O)NH—, H₂NC(O)—, (C₁-C₆alkyl)NHC(O)—, di(C₁-C₆alkyl)NC(O)—, —CN, hydroxyl, C₁-C₆alkoxy-, C₁-C₆alkyl-, HO₂C—, (C₁-C₆alkoxy)carbonyl-, —C(O)(C₁-C₆alkyl), C₆-C₁₄aryl-, C₁-C₉heteroaryl-, C₃-C₈cycloalkyl-, C₁-C₆haloalkyl-, C₁-C₆aminoalkyl-, (C₁-C₆alkyl)carboxy-, C₁-C₆carbonylamidoalkyl-, or O₂N—.

“Alkylcarboxy-” refers to an alkyl group, defined above, attached to the parent structure through the oxygen atom of a carboxyl (C(O)—O—) functionality. Examples of (C₁-C₆alkyl)carboxy- include acetoxy, ethylcarboxy, propylcarboxy, and isopentylcarboxy.

“(Alkyl)carbonylamido-” refers to a —NHC(O)— group in which the carbonyl carbon atom of said group is attached to a alkyl group, as defined above. Representative examples of a (C₁-C₆alkyl)carbonylamido- group include, but are not limited to, —NHC(O)CH₃, —NHC(O)CH₂CH₃, —NHC(O)CH₂CH₂CH₃, —NHC(O)CH₂CH₂CH₂CH₃, —NHC(O)CH₂CH₂CH₂CH₂CH₃, —NHC(O)CH(CH₃)₂, —NHC(O)CH₂CH(CH₃)₂, —NHC(O)CH(CH₃)CH₂CH₃, —NHC(O)—C(CH₃)₃ and —NHC(O)CH₂C(CH₃)₃.

“-Alkylene-”, “-alkenylene-”, and “-alkynylene-” refers to alkyl, alkenyl and alkynyl groups, as defined above, having two points of attachment within a chemical structure. Examples of —C₁-C₆alkylene- include ethylene (—CH₂CH₂—), propylene (—CH₂CH₂CH₂—), and dimethylpropylene (—CH₂C(CH₃)₂CH₂—). Likewise, examples of —C₂-C₆alkenylene- include ethenylene (—CH═CH— and propenylene (—CH═CH—CH₂—). Examples of —C₂-C₆alkynylene- include ethynylene (—C≡C—) and propynylene (—C≡C—CH₂—).

“Alkylthio-” refers to the group R—S— where R is an alkyl group, as defined above, attached to the parent structure through a sulfur atom. Examples of C₁-C₆alkylthio- include methylthio, ethylthio, n-propylthio, i-propylthio, n-butylthio, i-butylthio, s-butylthio, t-butylthio, n-pentylthio and n-hexylthio.

“Alkynyl-” refers to a straight or branched chain unsaturated hydrocarbon containing at least one triple bond. Examples of a C₂-C₆alkynyl- group include, but are not limited to, acetylene, propyne, 1-butyne, 2-butyne, isobutyne, sec-butyne, 1-pentyne, 2-pentyne, isopentyne, 1-hexyne, 2-hexyne, 3-hexyne, and isohexyne. An alkynyl group can be unsubstituted or substituted with one or more of the following groups: halogen, H₂N—, (C₁-C₆alkyl)amino-, di(C₁-C₆alkyl)amino-, (C₁-C₆alkyl)C(O)N(C₁-C₃alkyl)-, (C₁-C₆alkyl)carbonylamido-, HC(O)NH—, H₂NC(O)—, (C₁-C₆alkyl)NHC(O)—, di(C₁-C₆alkyl)NC(O)—, —CN, hydroxyl, C₁-C₆alkoxy-, C₁-C₆alkyl-, HO₂C—, (C₁-C₆alkoxy)carbonyl-, —C(O)(C₁-C₆alkyl), C₆-C₁₄aryl-, C₁-C₉heteroaryl-, and C₃-C₈cycloalkyl-.

“Amido(aryl)-” refers to an aryl group, as defined below, wherein one of the aryl group's hydrogen atoms has been replaced with one or more H₂NC(O)— groups. Representative examples of an amido(C₆-C₁₄aryl)- group include 2-C(O)NH₂-phenyl, 3-C(O)NH₂-phenyl, 4-C(O)NH₂-phenyl, 1-C(O)NH₂-naphthyl, and 2-C(O)NH₂-naphthyl.

“Aminoalkyl-” refers to an alkyl group, as defined above, wherein one or more of the alkyl group's hydrogen atoms has been replaced with H₂N—. Representative examples of an C₁-C₆aminoalkyl- group include, but are not limited to —CH₂NH₂, —CH₂CH₂NH₂, —CH₂CH₂CH₂NH₂, —CH₂CH₂CH₂CH₂NH₂, —CH₂CH(NH₂)CH₃, —CH₂CH(NH₂)CH₂CH₃, —CH(NH₂)CH₂CH₃, —C(CH₃)₂(CH₂NH₂), —CH₂CH₂CH₂CH₂CH₂NH₂, and —CH₂CH₂CH(NH₂)CH₂CH₃. An aminoalkyl group can be unsubstituted or substituted with one or two of the following groups: C₁-C₆alkoxy-, C₆-C₁₄aryl-, C₁-C₉heteroaryl-, C₃-C₈cycloalkyl-, and C₁-C₆alkyl-.

Aryl- refers to an aromatic hydrocarbon group. Examples of an C₆-C₁₄aryl- group include, but are not limited to, phenyl, 1-naphthyl, 2-naphthyl, 3-biphen-1-yl, anthryl, tetrahydronaphthyl, fluorenyl, indanyl, biphenylenyl, and acenaphthenyl. An aryl group can be unsubstituted or substituted with one or more of the following groups: C₁-C₆alkyl-, halogen, haloalkyl-, hydroxyl, hydroxyl(C₁-C₆alkyl)-, H₂N—, aminoalkyl-, di(C₁-C₆alkyl)amino-, HO₂C—, (C₁-C₆alkoxy)carbonyl-, (C₁-C₆alkyl)carboxy-, di(C₁-C₆alkyl)amido-, H₂NC(O)—, (C₁-C₆alkyl)amido-, or O₂N—.

“(Aryl)alkyl-” refers to an alkyl group, as defined above, wherein one or more of the alkyl group's hydrogen atoms has been replaced with an aryl group as defined above. (C₆-C₁₄Aryl)alkyl- moieties include benzyl, benzhydryl, 1-phenylethyl, 2-phenylethyl, 3-phenylpropyl, 2-phenylpropyl, 1-naphthylmethyl, 2-naphthylmethyl and the like. An (aryl)alkyl group can be unsubstituted or substituted with one or more of the following groups: halogen, H₂N—, hydroxyl, (C₁-C₆alkyl)amino-, di(C₁-C₆alkyl)amino-, (C₁-C₆alkyl)C(O)N(C₁-C₃alkyl)-, (C₁-C₆alkyl)carbonylamido-, HC(O)NH—, H₂NC(O)—, (C₁-C₆alkyl)NHC(O)—, di(C₁-C₆alkyl)NC(O)—, —CN, hydroxyl, C₁-C₆alkoxy-, C₁-C₆alkyl-, HO₂C—, (C₁-C₆alkoxy)carbonyl-, —C(O)(C₁-C₆alkyl), C₆-C₁₄aryl-, C₁-C₉heteroaryl-, C₃-C₈cycloalkyl-, C₁-C₆haloalkyl-, C₁-C₆aminoalkyl-, (C₁-C₆alkyl)carboxy-, C₁-C₆ carbonylamidoalkyl-, or O₂N—.

“(Aryl)amino-” refers to a radical of formula (aryl)-NH—, wherein aryl is as defined above. Examples of (C₆-C₁₄aryl)amino- radicals include, but are not limited to, phenylamino (anilido), 1-naphthlamino, 2-naphthlamino and the like. An (aryl)amino group can be unsubstituted or substituted with one or more of the following groups: halogen, H₂N—, (C₁-C₆alkyl)amino-, di(C₁-C₆alkyl)amino-, (C₁-C₆alkyl)C(O)N(C₁-C₃alkyl)-, (C₁-C₆alkyl)carbonylamido-, HC(O)NH—, H₂NC(O)—, (C₁-C₆alkyl)NHC(O)—, di(C₁-C₆alkyl)NC(O)—, —CN, hydroxyl, C₁-C₆alkoxy-, C₁-C₆alkyl-, HO₂C—, (C₁-C₆alkoxy)carbonyl-, —C(O)(C₁-C₆alkyl), C₆-C₁₄aryl-, C₁-C₉heteroaryl-, or C₃-C₈cycloalkyl-.

“(Aryl)oxy-” refers to the group Ar—O— where Ar is an aryl group, as defined above. Exemplary (C₆-C₁₄aryl)oxy- groups include but are not limited to phenyloxy, α-naphthyloxy, and β-naphthyloxy. An (aryl)oxy group can be unsubstituted or substituted with one or more of the following groups: C₁-C₆alkyl-, halogen, C₁-C₆haloalkyl-, hydroxyl, C₁-C₆hydroxylalkyl-, H₂N—, C₁-C₆aminoalkyl-, di(C₁-C₆alkyl)amino-, HO₂C—, (C₁-C₆alkoxy)carbonyl-, (C₁-C₆alkyl)carboxy-, di(C₁-C₆alkyl)amido-, H₂NC(O)—, (C₁-C₆alkyl)amido-, or O₂N—.

“Cycloalkyl-” refers to a monocyclic, non-aromatic, saturated hydrocarbon ring. Representative examples of a C₃-C₈cycloalkyl- include, but are not limited to, cyclopropyl, cyclobutyl, cyclopentyl, cyclohexyl, cycloheptyl, and cyclooctyl. A cycloalkyl can be unsubstituted or independently substituted with one or more of the following groups: halogen, H₂N—, (C₁-C₆alkyl)amino-, di(C₁-C₆alkyl)amino-, (C₁-C₆alkyl)C(O)N(C₁-C₃alkyl)-, (C₁-C₆alkyl)carbonylamido-, HC(O)NH—, H₂NC(O)-, (C₁-C₆alkyl)NHC(O)—, di(C₁-C₆alkyl)NC(O)—, —CN, hydroxyl, C₁-C₆alkoxy-, C₁-C₆alkyl-, HO₂C—, (C₁-C₆alkoxy)carbonyl-, —C(O)(C₁-C₆alkyl), C₆-C₁₄aryl-, C₁-C₉heteroaryl-, or C₃-C₈cycloalkyl-, C₁-C₆haloalkyl-, C₁-C₆aminoalkyl-, (C₁-C₆alkyl)carboxy-, C₁-C₆carbonylamidoalkyl-, or O₂N—. Additionally, each of any two hydrogen atoms on the same carbon atom of the carbocyclic ring can be replaced by an oxygen atom to form an oxo (═O) substituent or the two hydrogen atoms can be replaced by an alkylenedioxy group so that the alkylenedioxy group, when taken together with the carbon atom to which it is attached, form a 5- to 7-membered heterocycle containing two oxygen atoms.

“Bicyclic cycloalkyl-” refers to a bicyclic, non-aromatic, saturated hydrocarbon ring system. Representative examples of a C₆-C₁₀bicyclic cycloalkyl- include, but are not limited to, cis-1-decalinyl, trans 2-decalinyl, cis-4-perhydroindanyl, and trans-7-perhydroindanyl. A bicyclic cycloalkyl can be unsubstituted or independently substituted with one or more of the following groups: halogen, H₂N—, (C₁-C₆alkyl)amino-, di(C₁-C₆alkyl)amino-, (C₁-C₆alkyl)C(O)N(C₁-C₃alkyl)-, (C₁-C₆alkyl)carbonylamido-, HC(O)NH—, H₂NC(O)—, (C₁-C₆alkyl)NHC(O)—, di(C₁-C₆alkyl)NC(O)—, —CN, hydroxyl, C₁-C₆alkoxy-, C₁-C₆alkyl-, HO₂C—, (C₁-C₆alkoxy)carbonyl-, —C(O)(C₁-C₆alkyl), C₆-C₁₄aryl-, C₁-C₉heteroaryl-, or C₃-C₈cycloalkyl-, haloalkyl-, aminoalkyl-, (C₁-C₆alkyl)carboxy-, carbonylamidoalkyl-, or O₂N—. Additionally, each of any two hydrogen atoms on the same carbon atom of the bicyclic cycloalkyl- rings can be replaced by an oxygen atom to form an oxo (═O) substituent or the two hydrogen atoms can be replaced by an alkylenedioxy group so that the alkylenedioxy group, when taken together with the carbon atom to which it is attached, form a 5- to 7-membered heterocycle containing two oxygen atoms.

“Carboxyamidoalkyl-” refers to a primary carboxyamide (CONH₂), a secondary carboxyamide (CONHR′) or a tertiary carboxyamide (CONR′R″), where R′ and R″ are the same or different substituent groups selected from C₁-C₆alkyl-, C₂-C₆alkenyl, C₂-C₆alkynyl, C₆-C₁₄aryl-, C₁-C₉heteroaryl-, or C₃-C₈cycloalkyl-, attached to the parent compound by an —C₁-C₆alkylene- group as defined above. Exemplary C₁-C₆carbonylamidoalkyl- groups include but are not limited to NH₂C(O)—CH₂—, CH₃NHC(O)—CH₂CH₂—, (CH₃)₂NC(O)—CH₂CH₂CH₂—, CH₂═CHCH₂NHC(O)—CH₂CH₂CH₂CH₂—, HCCCH₂NHC(O)—CH₂CH₂CH₂CH₂CH₂—, C₆H₅NHC(O)—CH₂CH₂CH₂CH₂CH₂CH₂—, 3-pyridylNHC(O)—CH₂CH(CH₃)CH₂CH₂—, and cyclopropyl-CH₂NHC(O)—CH₂CH₂C(CH₃)₂CH₂—.

“Cycloalkenyl-” refers to non-aromatic carbocyclic rings with one or more carbon-to-carbon double bonds within the ring system. The “cycloalkenyl” may be a single ring or may be multi-ring. Multi-ring structures may be bridged or fused ring structures. Examples of C₃-C₁₀cycloalkenyl- groups include, but are not limited to, cyclopropenyl, cyclobutenyl, cyclopentenyl, cyclohexenyl, 4,4a-octalin-3-yl, and cyclooctenyl. A cycloalkenyl can be unsubstituted or independently substituted with one or more of the following groups: halogen, H₂N—, (C₁-C₆alkyl)amino-, di(C₁-C₆alkyl)amino-, (C₁-C₆alkyl)C(O)N(C₁-C₃alkyl)-, (C₁-C₆alkyl)carbonylamido-, HC(O)NH—, H₂NC(O)—, (C₁-C₆alkyl)NHC(O)—, di(C₁-C₆alkyl)NC(O)—, —CN, hydroxyl, C₁-C₆alkoxy-, C₁-C₆alkyl-, HO₂C—, (C₁-C₆alkoxy)carbonyl-, —C(O)(C₁-C₆alkyl), C₆-C₁₄aryl-, C₁-C₉heteroaryl-, or C₃-C₈cycloalkyl-, C₁-C₆haloalkyl-, C₁-C₆aminoalkyl-, (C₁-C₆alkyl)carboxy-, C₁-C₆carbonylamidoalkyl-, or O₂N— Additionally, each of any two hydrogen atoms on the same carbon atom of the cycloalkenyl rings may be replaced by an oxygen atom to form an oxo (═O) substituent or the two hydrogen atoms may be replaced by an alkylenedioxy group so that the alkylenedioxy group, when taken together with the carbon atom to which it is attached, form a 5- to 7-membered heterocycle containing two oxygen atoms.

“Di(alkyl)amido-” refers to a —NC(O)— group in which the nitrogen atom of said group is attached to two alkyl groups, as defined above. Each alkyl group can be independently selected. Representative examples of a di(C₁-C₆alkyl)amido- group include, but are not limited to, —C(O)N(CH₃)₂, —C(O)N(CH₂CH₃)₂, —C(O)N(CH₃)CH₂CH₃, —C(O)N(CH₂CH₂CH₂CH₃)₂, —C(O)N(CH₂CH₃)CH₂CH₂CH₃, —C(O)N(CH₃)CH(CH₃)₂, —C(O)N(CH₂CH₃)CH₂CH(CH₃)₂, —C(O)N(CH(CH₃)CH₂CH₃)₂, —C(O)N(CH₂CH₃)C(CH₃)₃ and —C(O)N(CH₂CH₃)CH₂C(CH₃)₃.

“Di(alkyl)amino-” refers to a nitrogen atom attached to two alkyl groups, as defined above. Each alkyl group can be independently selected. Representative examples of an di(C₁-C₆alkyl)amino- group include, but are not limited to, —N(CH₃)₂, —N(CH₂CH₃)(CH₃), —N(CH₂CH₃)₂, —N(CH₂CH₂CH₃)₂, —N(CH₂CH₂CH₂CH₃)₂, —N(CH(CH₃)₂)₂, —N(CH(CH₃)₂)(CH₃), —N(CH₂CH(CH₃)₂)₂, —NH(CH(CH₃)CH₂CH₃)₂, —N(C(CH₃)₃)₂, —N(C(CH₃)₃)(CH₃), and —N(CH₃)(CH₂CH₃). The two alkyl groups on the nitrogen atom, when taken together with the nitrogen to which they are attached, can form a 3- to 7-membered nitrogen containing heterocycle wherein up to two of the carbon atoms of the heterocycle can be replaced with —N(H)—, —N(C₁-C₆alkyl)-, —N(C₃-C₈cycloalkyl)-, —N(C₆-C₁₄aryl)-, —N(C₁-C₉heteroaryl)-, —N(C₁-C₆aminoalkyl)-, —N(C₆-C₁₄arylamino)-, —O—, —S—, —S(O)—, or —S(O)₂—.

“Halo” or “halogen” refers to fluorine, chlorine, bromine, or iodine.

“Haloalkyl-” refers to a alkyl group, as defined above, wherein one or more of the hydrogen atoms has been replaced with —F, —Cl, —Br, or —I. Each substitution can be independently selected. Representative examples of an C₁-C₆haloalkyl- group include, but are not limited to, —CH₂F, —CCl₃, —CF₃, CH₂CF₃, —CH₂Cl, —CH₂CH₂Br, —CH₂CH₂I, —CH₂CH₂CH₂F, —CH₂CH₂CH₂Cl, —CH₂CH₂CH₂CH₂Br, —CH₂CH₂CH₂CH₂I, —CH₂CH₂CH₂CH₂CH₂Br, —CH₂CH₂CH₂CH₂CH₂I, —CH₂CH(Br)CH₃, —CH₂CH(Cl)CH₂CH₃, —CH(F)CH₂CH₃ and —C(CH₃)₂(CH₂Cl).

“Heteroaryl-” refers to 5-10-membered mono and bicyclic aromatic groups containing at least one heteroatom selected from oxygen, sulfur and nitrogen. Examples of monocyclic C₁-C₉heteroaryl- radicals include, but are not limited to, oxazinyl, thiazinyl, diazinyl, triazinyl, thiadiazoyl, tetrazinyl, imidazolyl, tetrazolyl, isoxazolyl, furanyl, furazanyl, oxazolyl, thiazolyl, thiophenyl, pyrazolyl, triazolyl, pyrimidinyl, N-pyridyl, 2-pyridyl, 3-pyridyl and 4-pyridyl. Examples of bicyclic C₁-C₉heteroaryl- radicals include but are not limited to, benzimidazolyl, indolyl, isoquinolinyl, benzofuranyl, benzothiophenyl, indazolyl, quinolinyl, quinazolinyl, purinyl, benzisoxazolyl, benzoxazolyl, benzthiazolyl, benzodiazolyl, benzotriazolyl, isoindolyl, and indazolyl. The contemplated heteroaryl- rings or ring systems have a minumum of 5 members. Therefore, for example, C₁heteroaryl- radicals would include but are not limited to tetrazolyl, C₂heteroaryl- radicals include but are not limited to triazolyl, thiadiazoyl, and tetrazinyl, C₉heteroaryl- radicals include but are not limited to quinolinyl and isoquinolinyl. A heteroaryl group can be unsubstituted or substituted with one or more of the following groups: C₁-C₆alkyl-, halogen, C₁-C₆haloalkyl-, hydroxyl, C₁-C₆hydroxylalkyl-, H₂N—, C₁-C₆aminoalkyl-, di(C₁-C₆alkyl)amino-, —COOH, (C₁-C₆alkoxy)carbonyl-, (C₁-C₆alkyl)carboxy-, di(C₁-C₆alkyl)amido-, H₂NC(O)—, (C₁-C₆alkyl)amido-, or O₂N—.

“(Heteroaryl)alkyl-” refers to an alkyl group, as defined above, wherein one or more of the alkyl group's hydrogen atoms has been replaced with a heteroaryl- group as defined above. Examples of (C₁-C₉heteroaryl)alkyl- moieties include 2-pyridylmethyl, 2-thiophenylethyl, 3-pyridylpropyl, 2-quinolinylmethyl, 2-indolylmethyl, and the like. A (heteroaryl)alkyl group can be unsubstituted or substituted with one or more of the following groups: halogen, H₂N—, hydroxyl, (C₁-C₆alkyl)amino-, di(C₁-C₆alkyl)amino-, (C₁-C₆alkyl)C(O)N(C₁-C₃alkyl)-, (C₁-C₆alkyl)carbonylamido-, HC(O)NH—, H₂NC(O)—, (C₁-C₆alkyl)NHC(O)—, di(C₁-C₆alkyl)NC(O)—, —CN, hydroxyl, C₁-C₆alkoxy-, C₁-C₆alkyl-, HO₂C—, (C₁-C₆alkoxy)carbonyl-, —C(O)(C₁-C₆alkyl), C₆-C₁₄aryl-, C₁-C₉heteroaryl-, C₃-C₈cycloalkyl-, C₁-C₆haloalkyl-, C₁-C₆aminoalkyl-, (C₁-C₆alkyl)carboxy-, C₁-C₆carbonylamidoalkyl-, or O₂N—.

“(Heteroaryl)oxy-” refers to the group Het-O— where Het is a heteroaryl- group, as defined above. Exemplary (C₁-C₉heteroaryl)oxy- groups include but are not limited to pyridin-2-yloxy, pyridin-3-yloxy, pyrimidin-4-yloxy, and oxazol-5-yloxy. A (heteroaryl)oxy group can be unsubstituted or substituted with one or more of the following groups: C₁-C₆alkyl-, halogen, C₁-C₆haloalkyl-, hydroxyl, C₁-C₆hydroxylalkyl-, H₂N—, C₁-C₆aminoalkyl-, di(C₁-C₆alkyl)amino-, —COOH, (C₁-C₆alkoxy)carbonyl-, (C₁-C₆alkyl)carboxy-, di(C₁-C₆alkyl)amido-, H₂NC(O)—, (C₁-C₆alkyl)amido-, or O₂N—.

“Heteroatom” refers to a sulfur, nitrogen, or oxygen atom.

“Heterocycle” or “heterocyclyl-” refers to 3-10-membered monocyclic, fused bicyclic, and bridged bicyclic groups containing at least one heteroatom selected from oxygen, sulfur and nitrogen. A heterocycle may be saturated or partially saturated. Exemplary C₁-C₉heterocyclyl- groups include but are not limited to aziridine, oxirane, oxirene, thiirane, pyrroline, pyrrolidine, dihydrofuran, tetrahydrofuran, dihydrothiophene, tetrahydrothiophene, dithiolane, piperidine, 1,2,3,6-tetrahydropyridine-1-yl, tetrahydropyran, pyran, thiane, thiine, piperazine, oxazine, 5,6-dihydro-4H-1,3-oxazin-2-yl, 2,5-diazabicyclo[2.2.1]heptane, 2,5-diazabicyclo[2.2.2]octane, 3,6-diazabicyclo[3.1.1]heptane, 3,8-diazabicyclo[3.2.1]octane, 6-oxa-3,8-diazabicyclo[3.2.1]octane, 7-oxa-2,5-diazabicyclo[2.2.2]octane, 2,7-dioxa-5-azabicyclo[2.2.2]octane, 2-oxa-5-azabicyclo[2.2.1]heptane, 2-oxa-5-azabicyclo[2.2.2]octane, 3,6-dioxa-8-azabicyclo[3.2.1]octane, 3-oxa-6-azabicyclo[3.1.1]heptane, 3-oxa-8-azabicyclo[3.2.1]octane, 5,7-dioxa-2-azabicyclo[2.2.2]octane, 6,8-dioxa-3-azabicyclo[3.2.1]octane, 6-oxa-3-azabicyclo[3.1.1]heptane, 8-oxa-3-azabicyclo[3.2.1]octane, 8-oxa-3-azabicyclo[3.2.1]octan-3-yl, 2-methyl-2,5-diazabicyclo[2.2.1]heptane-5-yl, 1,3,3-trimethyl-6-azabicyclo[3.2.1]oct-6-yl, 4-methyl-3,4-dihydro-2H-1,4-benzoxazin-7-yl, thiazine, dithiane, and dioxane. The contemplated heterocycle rings or ring systems have a minimum of 3 members. Therefore, for example, C₁heterocyclyl- radicals would include but are not limited to oxaziranyl, diaziridinyl, and diazirinyl, C₂heterocyclyl- radicals include but are not limited to aziridinyl, oxiranyl, and diazetidinyl, C₉heterocyclyl- radicals include but are not limited to azecanyl, tetrahydroquinolinyl, and perhydroisoquinolinyl.

“Heterocyclyl(alkyl)-” refers to an alkyl group, as defined above, wherein one or more of the alkyl group's hydrogen atoms has been replaced with a heterocycle group as defined above. Heterocyclyl(C₁-C₆alkyl)- moieties include 2-pyridylmethyl, 1-piperazinylethyl, 4-morpholinylpropyl, 6-piperazinylhexyl, and the like. A heterocyclyl(alkyl) group can be unsubstituted or substituted with one or more of the following groups: halogen, H₂N—, (C₁-C₆alkyl)amino-, di(C₁-C₆alkyl)amino-, (C₁-C₆alkyl)C(O)N(C₁-C₃alkyl)-, (C₁-C₆alkyl)carbonylamido-, HC(O)NH—, H₂NC(O)—, (C₁-C₆alkyl)NHC(O)—, di(C₁-C₆alkyl)NC(O)—, —CN, hydroxyl, C₁-C₆alkoxy-, C₁-C₆alkyl-, HO₂C—, (C₁-C₆alkoxy)carbonyl-, —C(O)(C₁-C₆alkyl), 4- to 7-membered monocyclic heterocycle, C₆-C₁₄aryl-, C₁-C₉heteroaryl-, or C₃-C₈cycloalkyl-.

“Hydroxylalkyl-” refers to an alkyl group, as defined above, wherein one or more of the alkyl group's hydrogen atoms has been replaced with hydroxyl groups. Examples of C₁-C₆hydroxylalkyl- moieties include, for example, —CH₂OH, —CH₂CH₂OH, —CH₂CH₂CH₂OH, —CH₂CH(OH)CH₂OH, —CH₂CH(OH)CH₃, —CH(CH₃)CH₂OH and higher homologs.

“Hydroxylalkenyl-” refers to an alkenyl group, defined above, and substituted on one or more sp³ carbon atoms with a hydroxyl group. Examples of C₃-C₆hydroxylalkenyl- moieties include chemical groups such as —CH═CHCH₂OH, —CH(CH═CH₂)OH, —CH₂CH═CHCH₂OH, —CH(CH₂CH═CH₂)OH, —CH═CHCH₂CH₂OH, —CH(CH═CHCH₃)OH, —CH═CHCH(CH₃)OH, —CH₂CH(CH═CH₂)OH, and higher homologs.

“Leaving group” refers an atom or group (charged or uncharged) that becomes detached from an atom in what is considered to be the residual or main part of the substrate in a specified reaction. For example, in the heterolytic solvolysis of benzyl bromide in acetic acid: the leaving group is bromide. In the reaction of N,N,N-trimethyl-1-phenylmethanaminium ion with methanethiolate, the leaving group is trimethylamine. In the electrophilic nitration of benzene, it is H⁺. The term has meaning only in relation to a specified reaction. Examples of leaving groups include, for example, carboxylates (i.e. CH₃COO—, CF₃CO₂⁻), F⁻, water, Cl⁻, Br⁻, I⁻, N₃⁻, SCN⁻, trichloroacetimidate, thiopyridyl, tertiary amines (i.e. trimethylamine), phenoxides (i.e. nitrophenoxide), and sulfonates (i.e. tosylate, mesylate, triflate).

“Nitrogen-containing heteroaryl-” refers to 5-10-membered mono and bicyclic aromatic groups containing at least one nitrogen atom and optionally additional heteroatoms selected from oxygen and sulfur. Examples of nitrogen-containing monocyclic C₁-C₉heteroaryl- radicals include, but are not limited to, oxazinyl, thiazinyl, diazinyl, triazinyl, tetrazinyl, imidazolyl, tetrazolyl, isoxazolyl, furazanyl, oxazolyl, thiazolyl, pyrazolyl, triazolyl, pyrimidinyl, N-pyridyl, 2-pyridyl, 3-pyridyl and 4-pyridyl. Examples of nitrogen-containing bicyclic C₁-C₉heteroaryl- radicals include but are not limited to, benzimidazolyl, indolyl, isoquinolinyl, indazolyl, quinolinyl, quinazolinyl, purinyl, benzisoxazolyl, benzoxazolyl, benzthiazolyl, benzodiazolyl, benzotriazolyl, isoindolyl and indazolyl. A nitrogen-containing heteroaryl- group can be unsubstituted or substituted with one or more of the following groups: C₁-C₆alkyl-, halogen, C₁-C₆haloalkyl-, hydroxyl, C₁-C₆hydroxylalkyl-, H₂N—, C₁-C₆aminoalkyl-, di(C₁-C₆alkyl)amino-, HO₂C—, (C₁-C₆alkoxy)carbonyl-, (C₁-C₆alkyl)carboxy-, di(C₁-C₆alkyl)amido-, H₂NC(O)—, (C₁-C₆alkyl)amido-, or O₂N—.

“Perfluoroalkyl-” refers to alkyl group, defined above, having two or more fluorine atoms. Examples of a C₁-C₆perfluoroalkyl- group include CF₃, CH₂CF₃, CF₂CF₃ and CH(CF₃)₂.

The term “optionally substituted”, unless otherwise specified, as used herein means that at least one hydrogen atom of the optionally substituted group has been substituted with halogen, H₂N—, (C₁-C₆alkyl)amino-, di(C₁-C₆alkyl)amino-, (C₁-C₆alkyl)C(O)N(C₁-C₃alkyl)-, (C₁-C₆alkyl)carbonylamido-, HC(O)NH—, H₂NC(O)—, (C₁-C₆alkyl)NHC(O)—, di(C₁-C₆alkyl)NC(O)—, —CN, hydroxyl, C₁-C₆alkoxy-, C₁-C₆alkyl-, HO₂C—, (C₁-C₆alkoxy)carbonyl-, —C(O)(C₁-C₆alkyl), C₆-C₁₄aryl-, C₁-C₉heteroaryl-, or C₃-C₈cycloalkyl-.

An “effective amount” when used in connection a compound of the present invention of this invention is an amount effective for inhibiting mTOR or PI3K in a subject.

The term “reacting” is intended to represent bringing the chemical reactants together under conditions such to cause the chemical reaction indicated to take place.

A “subject” is a mammal, e.g., a human, mouse, rat, guinea pig, dog, cat, horse, cow, pig, or non-human primate, such as a monkey, chimpanzee, baboon or gorilla.

Representative “pharmaceutically acceptable salts” include but are not limited to, e.g., water-soluble and water-insoluble salts, such as the acetate, aluminum, amsonate (4,4-diaminostilbene-2,2-disulfonate), benzathine (N,N′-dibenzylethylenediamine), benzenesulfonate, benzoate, bicarbonate, bismuth, bisulfate, bitartrate, borate, bromide, butyrate, calcium, calcium edetate, camsylate (camphorsulfonate), carbonate, chloride, choline, citrate, clavulariate, diethanolamine, dihydrochloride, diphosphate, edetate, edisylate (camphorsulfonate), esylate (ethanesulfonate), ethylenediamine, fumarate, gluceptate (glucoheptonate), gluconate, glucuronate, glutamate, hexafluorophosphate, hexylresorcinate, hydrabamine (N,N′-bis(dehydroabietyl)ethylenediamine), hydrobromide, hydrochloride, hydroxynaphthoate, 1-hydroxy-2-naphthoate, 3-hydroxy-2-naphthoate, iodide, isothionate (2-hydroxyethanesulfonate), lactate, lactobionate, laurate, lauryl sulfate, lithium, magnesium, malate, maleate, mandelate, meglumine (1-deoxy-1-(methylamino)-D-glucitol), mesylate, methyl bromide, methylnitrate, methylsulfate, mucate, napsylate, nitrate, N-methylglucamine ammonium salt, oleate, oxalate, palmitate, pamoate (4,4′-methylenebis-3-hydroxy-2-naphthoate, or embonate), pantothenate, phosphate, picrate, polygalacturonate, potassium, propionate, p-toluenesulfonate, salicylate, sodium, stearate, subacetate, succinate, sulfate, sulfosaliculate, suramate, tannate, tartrate, teoclate (8-chloro-3,7-dihydro-1,3-dimethyl-1H-purine-2,6-dione), triethiodide, tromethamine (2-amino-2-(hydroxymethyl)-1,3-propanediol), valerate, and zinc salts.

Some compounds within the present invention possess one or more chiral centers, and the present invention includes each separate enantiomer of such compounds as well as mixtures of the enantiomers. Where multiple chiral centers exist in compounds of the present invention, the invention includes each combination as well as mixtures thereof. All chiral, diastereomeric, and racemic forms of a structure are intended, unless the specific stereochemistry or isomeric form is specifically indicated. It is well known in the art how to prepare optically active forms, such as by resolution of racemic forms or by synthesis from optically active starting materials.

The compounds of the present invention exhibit an mTOR inhibitory activity and, therefore, can be utilized to inhibit abnormal cell growth in which mTOR plays a role. Thus, the compounds of the present invention are effective in the treatment of disorders with which abnormal cell growth actions of mTOR are associated, such as restenosis, atherosclerosis, bone disorders, arthritis, diabetic retinopathy, psoriasis, benign prostatic hypertrophy, atherosclerosis, inflammation, angiogenesis, immunological disorders, pancreatitis, kidney disease, cancer, etc. In particular, the compounds of the present invention possess excellent cancer cell growth inhibiting effects and are effective in treating cancers, preferably all types of solid cancers and malignant lymphomas, and especially, leukemia, skin cancer, bladder cancer, breast cancer, uterus cancer, ovary cancer, prostate cancer, lung cancer, colon cancer, pancreas cancer, renal cancer, gastric cancer, brain tumor, advanced renal cell carcinoma, acute lymphoblastic leukemia, malignant melanoma, soft-tissue or bone sarcoma, etc.

The compounds of the present invention exhibit a PI3 kinase inhibitory activity and, therefore, can be utilized in order to inhibit abnormal cell growth in which PI3 kinases play a role. Thus, the compounds of the present invention are effective in the treatment of disorders with which abnormal cell growth actions of PI3 kinases are associated, such as restenosis, atherosclerosis, bone disorders, arthritis, diabetic retinopathy, psoriasis, benign prostatic hypertrophy, atherosclerosis, inflammation, angiogenesis, immunological disorders, pancreatitis, kidney disease, cancer, etc. In particular, the compounds of the present invention possess excellent cancer cell growth inhibiting effects and are effective in treating cancers, preferably all types of solid cancers and malignant lymphomas, and especially, leukemia, skin cancer, bladder cancer, breast cancer, uterus cancer, ovary cancer, prostate cancer, lung cancer, colon cancer, pancreas cancer, renal cancer, gastric cancer, brain tumor, advanced renal cell carcinoma, acute lymphoblastic leukemia, malignant melanoma, soft-tissue or bone sarcoma, etc.

For therapeutic use, the pharmacologically active compounds of Formula I will normally be administered as a pharmaceutical composition comprising as the (or an) essential active ingredient at least one such compound in association with a solid or liquid pharmaceutically acceptable carrier and, optionally, with pharmaceutically acceptable adjutants and excipients employing standard and conventional techniques.

The pharmaceutical compositions of this invention include suitable dosage forms for oral, parenteral (including subcutaneous, intramuscular, intradermal and intravenous) bronchial or nasal administration. Thus, if a solid carrier is used, the preparation may be tableted, placed in a hard gelatin capsule in powder or pellet form, or in the form of a troche or lozenge. The solid carrier may contain conventional excipients such as binding agents, fillers, tableting lubricants, disintegrants, wetting agents and the like. The tablet may, if desired, be film coated by conventional techniques. If a liquid carrier is employed, the preparation may be in the form of a syrup, emulsion, soft gelatin capsule, sterile vehicle for injection, an aqueous or non-aqueous liquid suspension, or may be a dry product for reconstitution with water or other suitable vehicle before use. Liquid preparations may contain conventional additives such as suspending agents, emulsifying agents, wetting agents, non-aqueous vehicle (including edible oils), preservatives, as well as flavoring and/or coloring agents. For parenteral administration, a vehicle normally will comprise sterile water, at least in large part, although saline solutions, glucose solutions and like may be utilized. Injectable suspensions also may be used, in which case conventional suspending agents may be employed. Conventional preservatives, buffering agents and the like also may be added to the parenteral dosage forms. Particularly useful is the administration of a compound of Formula I directly in parenteral formulations. The pharmaceutical compositions are prepared by conventional techniques appropriate to the desired preparation containing appropriate amounts of the active ingredient, that is, the compound of Formula I according to the invention. See, for example, Remington: The Science and Practice of Pharmacy, 20th Edition. Baltimore, Md.: Lippincott Williams & Wilkins, 2000.

The dosage of the compounds of Formula I to achieve a therapeutic effect will depend not only on such factors as the age, weight and sex of the patient and mode of administration, but also on the degree of potassium channel activating activity desired and the potency of the particular compound being utilized for the particular disorder of disease concerned. It is also contemplated that the treatment and dosage of the particular compound may be administered in unit dosage form and that one skilled in the art would adjust the unit dosage form accordingly to reflect the relative level of activity. The decision as to the particular dosage to be employed (and the number of times to be administered per day is within the discretion of the physician, and may be varied by titration of the dosage to the particular circumstances of this invention to produce the desired therapeutic effect.

A suitable dose of a compound of Formula I or pharmaceutical composition thereof for a mammal, including man, suffering from, or likely to suffer from any condition as described herein is an amount of active ingredient from about 0.01 .mg/kg to 10 mg/kg body weight. For parenteral administration, the dose may be in the range of 0.1 .mg/kg to 1 mg/kg body weight for intravenous administration. For oral administration, the dose may be in the range about 0.1 mg/kg to 5 mg/kg body weight. The active ingredient will preferably be administered in equal doses from one to four times a day. However, usually a small dosage is administered, and the dosage is gradually increased until the optimal dosage for the host under treatment is determined.

However, it will be understood that the amount of the compound actually administered will be determined by a physician, in the light of the relevant circumstances including the condition to be treated, the choice of compound of be administered, the chosen route of administration, the age, weight, and response of the individual patient, and the severity of the patient's symptoms.

The amount of the compound of the present invention or a pharmaceutically acceptable salt thereof that is effective for inhibiting mTOR or PI3K in a subject. In addition, in vitro or in vivo assays can optionally be employed to help identify optimal dosage ranges. The precise dose to be employed can also depend on the route of administration, the condition, the seriousness of the condition being treated, as well as various physical factors related to the individual being treated, and can be decided according to the judgment of a health-care practitioner. Equivalent dosages may be administered over various time periods including, but not limited to, about every 2 hours, about every 6 hours, about every 8 hours, about every 12 hours, about every 24 hours, about every 36 hours, about every 48 hours, about every 72 hours, about every week, about every two weeks, about every three weeks, about every month, and about every two months. The number and frequency of dosages corresponding to a completed course of therapy will be determined according to the judgment of a health-care practitioner. The effective dosage amounts described herein refer to total amounts administered; that is, if more than one compound of the present invention or a pharmaceutically acceptable salt thereof is administered, the effective dosage amounts correspond to the total amount administered.

In one embodiment, the compound of the present invention or a pharmaceutically acceptable salt thereof is administered concurrently with another therapeutic agent.

In one embodiment, a composition comprising an effective amount of a compound of the present invention or a pharmaceutically acceptable salt thereof and an effective amount of another therapeutic agent within the same composition can be administered.

Effective amounts of the other therapeutic agents are well known to those skilled in the art. However, it is well within the skilled artisan's purview to determine the other therapeutic agent's optimal effective amount range. The compound of the present invention or a pharmaceutically acceptable salt thereof and the other therapeutic agent can act additively or, in one embodiment, synergistically. In one embodiment, of the invention, where another therapeutic agent is administered to an animal, the effective amount of the compound of the present invention or a pharmaceutically acceptable salt thereof is less than its effective amount would be where the other therapeutic agent is not administered. In this case, without being bound by theory, it is believed that the compound of the present invention or a pharmaceutically acceptable salt thereof and the other therapeutic agent act synergistically.

The following abbreviations are used herein and have the indicated definitions: ACN is acetonitrile and AcOH is acetic acid. ATP is adenosine triphosphate. Biotage Initiator™ 60 is a 60-position sample microwave synthesizer. Initiator™ is a registered trademark of Biotage AB, Uppsala, Sweden. BOC is t-butoxycarbonyl. Celite™ is flux-calcined diatomaceous earth. Celite™ is a registered trademark of World Minerals Inc. CHAPS is (3-[(3-cholamidopropyl)dimethylammonio]-1-propanesulfonic acid, DEAD is diethyl azodicarboxylate, DIAD is diisopropylazodicarboxylate, DMAP is dimethyl aminopyridine, DME is 1,2-dimethoxyethane, DMF is N,N-dimethylformamide, DMF-DMA is dimethylformamide dimethyl acetal, and DMSO is dimethylsulfoxide. DPBS is Dulbecco's Phosphate Buffered Saline Formulation. EDCI is 1-ethyl-3-(3-dimethylaminopropyl)carbodiimide or water-soluble carbodiimide, EDTA is ethylenediaminetetraacetic acid, ESI stands for Electrospray Ionization, EtOAc is ethyl acetate, and EtOH is ethanol. HBTU is O-benzotriazole-N,N,N′,N′-tetramethyl-uronium-hexafluoro-phosphate, HEPES is 4-(2-hydroxyethyl)-1-piperazineethanesulfonic acid, GMF is glass microfiber, HOBT is N-hydroxybenzotriazole, Hunig's Base is diisopropylethylamine, HPLC is high-pressure liquid chromatography, LPS is lipopolysaccharide. MeCN is acetonitrile, MeOH is methanol, MS is mass spectrometry, and NEt₃ is triethylamine. Ni(Ra) is Raney™ nickel, a sponge-metal catalyst produced when a block of nickel-aluminum alloy is treated with concentrated sodium hydroxide. Raney™ is a registered trademark of W. R. Grace and Company. NMP is N-methylpyrrolidone, NMR is nuclear magnetic resonance, PBS is phosphate-buffered saline (pH 7.4), RPMI 1640 is a buffer (Sigma-Aldrich Corp., St. Louis, Mo., USA), SDS is dodecyl sulfate (sodium salt), SRB is Sulforhodamine B, TCA is trichloroacetic acid, TFA is trifluoroacetic acid, THF is tetrahydrofuran, THP is tetrahydro-2H-pyran-2-yl. TLC is thin-layer chromatography and TRIS is tris(hydroxymethyl)aminomethane.

### Methods

The following methods outline the synthesis of the compounds of Formula I. The following examples are presented to illustrate certain embodiments of the present invention, but should not be construed as limiting the scope of this invention.

### EXAMPLE 1

### Preparation of [3-(4-morpholin-4-yl-7H-pyrrolo[2,3-d]pyrimidin-2-yl)phenyl]methanol

### Step 1: Synthesis of 7H-pyrrolo[2,3-d]pyrimidine-2,4-diol

To a suspended solution of 6-aminouracil (12.7 g, 100 mmol) and sodium acetate (8.2 g, 100 mmol) in H₂O (100 mL) at a temperature of 70-75° C., was added a solution of chloroacetaldehyde (50% in water, 23.6 g, 150 mmol). The resulting reaction mixture was stirred at 80° C. for 20 min, and then cooled to room temperature. The separated solid was collected by filtration, washed with water and acetone, and dried in vacuum to give the title compound as brown solid (14.74 g, 98% yield). MS(ESI, M−1) m/z 150.2.

### Step 2: Synthesis of 2,4-dichloro-7H-pyrrolo[2,3-d]pyrimidine

To a 20 mL vial were added 7H-pyrrolo[2,3-d]pyrimidine-2,4-diol (2.5 g, 16.6 mmol), POCl₃ (10 mL, 107 mmol) and N,N-dimethylaniline (1 mL, 7.9 mmol). The resulting mixture was heated at 120° C. for 30 min in microwave oven. The reaction mixture was cooled to room temperature, and poured into ice, and neutralized by the addition of concentrated ammonium hydroxide to pH 5-7. The resulting solid was filtered, and washed with water to give the title compound as brown solid (1.323 g, 43% yield). MS(ESI, M+1) m/z 188.2.

### Step 3: Synthesis of 2-chloro-4-morpholin-4-yl-7H-pyrrolo[2,3-d]pyrimidine

To a solution of 2,4-dichloro-7H-pyrrolo[2,3-d]pyrimidine (1.38 g, 7.4 mmol) in CH₂Cl₂ (30 mL) were added morpholine (0.96 mL, 11 mmol) and Et₃N (2.1 mL, 15 mmol). The mixture was stirred at room temperature overnight. The resulting solid was filtered, washed with EtOH and water to give the title compound as yellow solid (1.19 g, 68%). MS(ESI) m/z 239.3

### Step 4: Synthesis of [3-(4-morpholin-4-yl-7H-pyrrolo[2,3-d]pyrimidin-2-yl)phenyl]methanol

To a 10 mL vial were added 2-chloro-4-morpholin-4-yl-7H-pyrrolo[2,3-d]pyrimidine (150 mg, 0.63 mmol), 3-hydroxymethylphenylboronic acid (144 mg, 0.94 mmol), Pd(PPh₃)₄ (36 mg, 5 mol %), 1,2-dimethoxyethane (DME, 2.5 mL) and saturated sodium bicarbonate aqueous solution (1.5 mL). The resulting mixture was heated at 120° C. for 1 h in microwave oven. The reaction mixture was cooled to room temperature. The aqueous phase was extracted with EtOAc, and the combined organic solution was concentrated under reduced pressure. The residue was subjected to HPLC separation to give the title compound as off-white solid (98 mg, 50% yield). MS(ESI) m/z 311.3. HRMS: calcd for C₁₇H₁₈N₄O₂+H⁺, 311.15025; found (ESI-FTMS, [M+H]¹⁺), 311.15016.

### EXAMPLE 2

### Preparation of 3-(4-morpholin-4-yl-7H-pyrrolo[2,3-d]pyrimidin-2-yl)phenol

Following the procedure as described as in Example 1, Suzuki coupling of 2-chloro-4-morpholin-4-yl-7H-pyrrolo[2,3-d]pyrimidine (150 mg, 0.63 mmol) with 3-hydroxyphenylboronic acid (130 mg, 0.94 mmol) gave the title compound as yellow solid (130 mg, 70% yield). MS(ESI) m/z 297.2. HRMS: calcd for C₁₆H₁₆N₄O₂+H⁺, 297.13460; found (ESI-FTMS, [M+H]¹⁺), 297.13471.

### EXAMPLE 3

### Preparation of 2-(1H-indazol-4-yl)-4-morpholin-4-yl-7H-pyrrolo[2,3-d]pyrimidine

Following the procedure as described as in Example 1, Suzuki coupling of 2-chloro-4-morpholin-4-yl-7H-pyrrolo[2,3-d]pyrimidine (14 mg, 0.06 mmol) with 1H-indazol-4-ylboronic acid pinacol ester (24 mg, 0.1 mmol) gave the title compound as yellow solid (6 mg, 32% yield). MS(ESI) m/z 321.3.

### EXAMPLE 4

### Preparation of 1-[4-(4-morpholin-4-yl-7H-pyrrolo[2,3-d]pyrimidin-2-yl)phenyl]-3-pyridin-4-ylurea

To a 10 mL vial were charged 4-isocyantophenylboronic acid pinacol ester (368 mg, 1.5 mmol), 4-aminopyridine (188 mg, 2.0 mmol), Et₃N (0.28 mL, 2.0 mmol) and DME (3 mL). The mixture was stirred at room temperature for 5 h, and then added 2-chloro-4-morpholin-4-yl-7H-pyrrolo[2,3-d]pyrimidine (238 mg, 1.0 mmol) and sodium carbonate aqueous solution (2M, 2 mL) and Pd(PPh₃)₄ (58 mg, 5 mol %). The resulting mixture was heated at 125° C. for 30 min in microwave oven, and cooled to room temperature. The aqueous phase was extracted with EtOAc, and the combined organic solution was concentrated under reduced pressure. The residue was subjected to HPLC separation to give the title compound as yellow solid (66 mg, 16% yield). MS(ESI) m/z 416.2. HRMS: calcd for C₂₂H₂₁N₇O₂+H⁺, 416.18295; found (ESI, [M+H]⁺ Calc'd), 416.1830.

### EXAMPLE 5

### Preparation of 1-[4-(4-morpholin-4-yl-7H-pyrrolo[2,3-d]pyrimidin-2-yl)phenyl]-3-pyridin-3-ylurea

Following the procedure described in Example 4, using 3-aminopyridine (188 mg, 1.5 mmol) instead of 4-aminopyrimidine, the title compound was isolated as off-white solid (89 mg, 21% yield). MS(ESI) m/z 416.2.

### EXAMPLE 6

### Preparation of 3-{7-[2-(dimethylamino)ethyl]-4-morpholin-4-yl-7H-pyrrolo[2,3-d]pyrimidin-2-yl}phenol

### Step 1: Synthesis of 2-chloro-4-morpholin-4-yl-7-[2-(dimethylamino)ethyl]-7H-pyrrolo[2,3-d]pyrimidine

To a solution of 2-chloro-4-morpholin-4-yl-7H-pyrrolo[2,3-d]pyrimidine (154 mg, 0.65 mmol) in DMF (5 mL) were added 2-(dimethylamino)ethyl chloride hydrochloride (140 mg, 0.97 mmol) and Cs₂CO₃ (635 mg, 1.95 mmol). The resulting mixture was heated at 80° C. under nitrogen overnight, and cooled to room temperature. Water was added, and the mixture was extracted with EtOAc. The combined extracts were washed with water and brine, dried over MgSO₄. The solvent was removed under reduced pressure to give the title compound as yellow syrup (169 mg, 84% yield), which was used in next step without further purification. MS(ESI) m/z 310.3.

### Step 2: Synthesis of 3-{7-[2-(dimethylamino)ethyl]-4-morpholin-4-yl-7H-pyrrolo[2,3-d]pyrimidin-2-yl}phenol

To a 10 mL vial were added 2-chloro-4-morpholin-4-yl-7-[2-(dimethylamino)ethyl]-7H-pyrrolo[2,3-d]pyrimidine (80 mg, 0.26 mmol), 3-hydroxyphenylboronic acid (54 mg, 0.38 mmol), Pd(PPh₃)₄ (15 mg, 5 mol %), 1,2-dimethoxyethane (DME, 3 mL) and sodium carbonate aqueous solution (2M, 2 mL). The resulting mixture was heated at 150° C. for 40 min in microwave oven, and then cooled to room temperature. The aqueous phase was extracted with EtOAc, and the combined organic solution was concentrated under reduced pressure. The residue was subjected to HPLC separation to give the title compound as off-white solid (81.9 mg, 78% yield). MS(ESI) m/z 368.4.

### EXAMPLE 7

### Preparation of (3-{7-[2-(dimethylamino)ethyl]-4-morpholin-4-yl-7H-pyrrolo[2,3-d]pyrimidin-2-yl}phenyl)methanol

Following the procedure as described in Example 6, reaction of 2-chloro-4-morpholin-4-yl-7-[2-(dimethylamino)ethyl]-7H-pyrrolo[2,3-d]pyrimidine (80 mg, 0.26 mmol) and 3-hydroxymethylphenylboronic acid (58 mg, 0.38 mmol) gave the title compound as off-white solid (96 mg, 88% yield). MS(ESI) m/z 382.4.

### EXAMPLE 8

### Preparation of 4-{7-[2-(dimethylamino)ethyl]-4-morpholin-4-yl-7H-pyrrolo[2,3-d]pyrimidin-2-yl}aniline

Following the procedure described in Example 6, reaction of 2-chloro-4-morpholin-4-yl-7-[2-(dimethylamino)ethyl]-7H-pyrrolo[2,3-d]pyrimidine (261 mg, 0.84 mmol) and 4-aminophenylboronic acid pinacol ester (277 mg, 1.27 mmol) gave the title compound as yellow oil (278 mg, 90% yield). MS(ESI) m/z 367.2.

### EXAMPLE 9

### Preparation of 1-(4-{7-[2-(dimethylamino)ethyl]-4-morpholin-4-yl-7H-pyrrolo[2,3-d]pyrimidin-2-yl}phenyl)-3-pyridin-3-ylurea

To a solution of 4-{7-[2-(dimethylamino)ethyl]-4-morpholin-4-yl-7H-pyrrolo[2,3-d]pyrimidin-2-yl}aniline (22 mg, 0.06 mmol) in CHCl₃ (1 mL) were added Et₃N (25 μL, 0.18 mmol) and triphosgene (18 mg, 0.06 mmol). The mixture was stirred at room temperature for 15 min and 3-aminopyridine (17 mg, 0.18 mmol) was added. The mixture was stirred at room temperature overnight. The solvent was removed, and the residue was subjected to HPLC separation to give the title compound as off-white solid (13 mg, 45% yield). MS(ESI) m/z 487.2.

### EXAMPLE 10

### Preparation of 7-[2-(dimethylamino)ethyl]-4-morpholin-4-yl-N-pyridin-3-yl-2-{4-[(pyridin-3-ylcarbamoyl)amino]phenyl}-7H-pyrrolo[2,3-d]pyrimidine-5-carboxamide

The titled compound was isolated as a by-product in Example 9 as off-white solid (8 mg, 22% yield). MS(ESI) m/z 607.5. HRMS: calcd for C₃₂H₃₄N₁₀O₃+H⁺, 607.28881; found (ESI, [M+H]⁺ Calc'd), 607.2888.

### EXAMPLE 11

### Preparation of 1-(4-{7-[2-(dimethylamino)ethyl]-4-morpholin-4-yl-7H-pyrrolo[2,3-d]pyrimidin-2-yl}phenyl)-3-pyridin-2-ylurea

Following the procedure described in Example 9, reaction of 4-{7-[2-(dimethylamino)ethyl]-4-morpholin-4-yl-7H-pyrrolo[2,3-d]pyrimidin-2-yl}aniline (22 mg, 0.06 mmol, triphosgene (18 mg, 0.06 mmol) and 2-aminopyridine (17 mg, 0.18 mmol) gave the title compound as off-white solid (15 mg, 51% yield). MS(ESI) m/z 487.3.

### EXAMPLE 12

### Preparation of 1-(4-{7-[2-(dimethylamino)ethyl]-4-morpholin-4-yl-7H-pyrrolo[2,3-d]pyrimidin-2-yl}phenyl)-3-pyridin-4-ylurea

Following the procedure described in Example 9, reaction of 4-{7-[2-(dimethylamino)ethyl]-4-morpholin-4-yl-7H-pyrrolo[2,3-d]pyrimidin-2-yl}aniline (22 mg, 0.06 mmol, triphosgene (18 mg, 0.06 mmol) and 4-aminopyridine (17 mg, 0.18 mmol) gave the title compound as off-white solid (18 mg, 62% yield). MS(ESI) m/z 487.3.

### EXAMPLE 13

### Preparation of 1-(4-{7-[2-(dimethylamino)ethyl]-4-morpholin-4-yl-7H-pyrrolo[2,3-d]pyrimidin-2-yl}phenyl)-3-(4-fluorophenyl)urea

Following the procedure described in Example 9, reaction of 4-{7-[2-(dimethylamino)ethyl]-4-morpholin-4-yl-7H-pyrrolo[2,3-d]pyrimidin-2-yl}aniline (22 mg, 0.06 mmol, triphosgene (18 mg, 0.06 mmol) and 4-fluoroaniline (20 mg, 0.18 mmol) gave the title compound as off-white solid (10 mg, 33% yield). MS(ESI) m/z 504.5.

### EXAMPLE 14

### Preparation of 1-[2-(dimethylamino)ethyl]-3-(4-{7-[2-(dimethylamino)ethyl]-4-morpholin-4-yl-7H-pyrrolo[2,3-d]pyrimidin-2-yl}phenyl)urea

Following the procedure described in Example 9, reaction of 4-{7-[2-(dimethylamino)ethyl]-4-morpholin-4-yl-7H-pyrrolo[2,3-d]pyrimidin-2-yl}aniline (22 mg, 0.06 mmol, triphosgene (18 mg, 0.06 mmol) and N,N-dimethylethylenediamine (16 mg, 0.18 mmol) gave the title compound as yellow solid (25 mg, 60% yield). MS(ESI) m/z 481.5.

### EXAMPLE 15

### Preparation of 1-(4-{7-[2-(dimethylamino)ethyl]-4-morpholin-4-yl-7H-pyrrolo[2,3-d]pyrimidin-2-yl}phenyl)-3-[3-(dimethylamino)propyl]urea

Following the procedure described in Example 9, reaction of 4-{7-[2-(dimethylamino)ethyl]-4-morpholin-4-yl-7H-pyrrolo[2,3-d]pyrimidin-2-yl}aniline (22 mg, 0.06 mmol, triphosgene (18 mg, 0.06 mmol) and 3-(dimethylamino)-1-propylamine (18 mg, 0.18 mmol) gave the title compound as yellow solid (27 mg, 67% yield). MS(ESI) m/z 495.6.

### EXAMPLE 16

### Preparation of 1-(4-{7-[2-(dimethylamino)ethyl]-4-morpholin-4-yl-7H-pyrrolo[2,3-d]pyrimidin-2-yl}phenyl)-3-ethylurea

Following the procedure described in Example 9, reaction of 4-{7-[2-(dimethylamino)ethyl]-4-morpholin-4-yl-7H-pyrrolo[2,3-d]pyrimidin-2-yl}aniline (22 mg, 0.06 mmol, triphosgene (18 mg, 0.06 mmol) and ethylamine (2M in THF, 0.18 mL, 0.36 mmol) gave the title compound as yellow solid (13.6 mg, 41% yield). MS(ESI) m/z 438.3.

### EXAMPLE 17

### Preparation of 1-(4-{7-[2-(dimethylamino)ethyl]-4-morpholin-4-yl-7H-pyrrolo[2,3-d]pyrimidin-2-yl}phenyl)-3-methylurea

Following the procedure described in Example 9, reaction of 4-{7-[2-(dimethylamino)ethyl]-4-morpholin-4-yl-7H-pyrrolo[2,3-d]pyrimidin-2-yl}aniline (22 mg, 0.06 mmol, triphosgene (18 mg, 0.06 mmol) and methylamine (2M in THF, 0.18 mL, 0.36 mmol) gave the title compound as yellow solid (11.1 mg, 34% yield). MS(ESI) m/z 424.4.

### EXAMPLE 18

### Preparation of 1-(4-{7-[2-(dimethylamino)ethyl]-4-morpholin-4-yl-7H-pyrrolo[2,3-d]pyrimidin-2-yl}phenyl)-3-[2-(1H-indol-3-yl)ethyl]urea

Following the procedure described in Example 9, reaction of 4-{7-[2-(dimethylamino)ethyl]-4-morpholin-4-yl-7H-pyrrolo[2,3-d]pyrimidin-2-yl}aniline (22 mg, 0.06 mmol, triphosgene (18 mg, 0.06 mmol) and tryptamine (29 mg, 0.18 mmol) gave the title compound as yellow solid (15.2 mg, 32% yield). MS(ESI) m/z 553.5.

### EXAMPLE 19

### Preparation of 1-[3-({2-[3-(hydroxymethyl)phenyl]-4-morpholin-4-yl-7H-pyrrolo[2,3-d]pyrimidin-7-yl}methyl)phenyl]urea

### Step 1: Synthesis of 2-chloro-4-morpholin-4-yl-7-(3-nitrobenzyl)-7H-pyrrolo[2,3-d]pyrimidine

To a solution of 2-chloro-4-morpholin-4-yl-7H-pyrrolo[2,3-d]pyrimidine (400 mg, 1.7 mmol) in DMF (15 mL) were added 3-nitrobenzyl bromide (545 mg, 2.5 mmol) and Cs₂CO₃ (1.095 g, 3.4 mmol). The resulting mixture was heated at 80° C. under nitrogen overnight, and cooled to room temperature. Water was added, and the mixture was extracted with EtOAc. The combined extracts were washed with water and brine, dried over MgSO₄. The solvent was removed under reduced pressure to give the title compound as yellow solid (533 mg, 84% yield), which was used in next step without further purification. MS(ESI) m/z 374.3.

### Step 2: Synthesis of 3-[(2-chloro-4-morpholin-4-yl-7H-pyrrolo[2,3-d]pyrimidin-7-yl)methyl]aniline

To a solution of 2-chloro-4-morpholin-4-yl-7-(3-nitrobenzyl)-7H-pyrrolo[2,3-d]pyrimidine (140 mg, 0.38 mmol) in MeOH (20 mL) was added Raney-Ni (420 mg), followed by addition of hydrazine (94 mg, 1.9 mmol). The resulting mixture was vigorously stirred at room temperature for 4 h, and filtered through a pad of Celite, washed with MeOH. The filtration was concentrated under reduced pressure, and the resulting solid was collected by filtration and washed with ether to give the title compound as yellow solid (116 mg, 90% yield). MS(ESI) m/z 344.4.

### Step 3: Synthesis of 1-(3-((2-chloro-4-morpholin-4-yl-7H-pyrrolo[2,3-d]pyrimidin-7-yl)methyl)phenyl)urea

To a solution of 3-[(2-chloro-4-morpholin-4-yl-7H-pyrrolo[2,3-d]pyrimidin-7-yl)methyl]aniline (170 mg, 0.5 mmol) in THF (5 mL) were added Et3N (0.2 mL, 1.5 mmol) and triphosgene (158 mg, 0.5 mmol). The resulting mixture was stirred at room temperature for 15 min before ammonium hydroxide (30% in water, 0.36 mL, 3 mmol) was added. The mixture was stirred at room temperature for 20 min, and concentrated in vacuum. The residue was subjected to HPLC separation to give the title compound as off-white solid (125 mg, 65% yield). MS(ESI) m/z 387.2.

### Step 4: Synthesis of 1-[3-({2-[3-(hydroxymethyl)phenyl]-4-morpholin-4-yl-7H-pyrrolo[2,3-d]pyrimidin-7-yl}methyl)phenyl]urea

To a 10 mL vial were added 1-(3-((2-chloro-4-morpholin-4-yl-7H-pyrrolo[2,3-d]pyrimidin-7-yl)methyl)phenyl)urea (50 mg, 0.13 mmol), 3-hydroxymethylphenylboronic acid (30 mg, 0.19 mmol), Pd(PPh₃)₄ (8 mg, 5 mol %), 1,2-dimethoxyethane (DME, 2 mL) and sodium carbonate aqueous solution (2M, 1 mL). The resulting mixture was heated at 130° C. for 30 min in microwave oven, and then cooled to room temperature. The aqueous phase was extracted with EtOAc, and the combined organic solution was concentrated under reduced pressure. The residue was subjected to HPLC separation to give the title compound as off-white solid (9.4 mg, 16% yield). MS(ESI) m/z 459.7.

### EXAMPLE 20

### Preparation of 1-(4-{7-[3-(carbamoylamino)benzyl]-4-morpholin-4-yl-7H-pyrrolo[2,3-d]pyrimidin-2-yl}phenyl)-3-pyridin-4-ylurea

Following the procedure described in Example 19. To a 10 mL vial were charged 4-isocyantophenylboronic acid pinacol ester (109 mg, 0.44 mmol), 4-aminopyridine (55 mg, 0.6 mmol), Et₃N (0.12 mL, 0.9 mmol) and DME (2 mL). The mixture was stirred at room temperature for 5 h, and then added 1-(3-((2-chloro-4-morpholin-4-yl-7H-pyrrolo[2,3-d]pyrimidin-7-yl)methyl)phenyl)urea (114 mg, 0.3 mmol) and sodium carbonate aqueous solution (2M, 1 mL) and Pd(PPh₃)₄ (17 mg, 5 mol %). The resulting mixture was heated at 130° C. for 30 min in microwave oven and then cooled to room temperature. The aqueous phase was extracted with EtOAc, and the combined organic solution was concentrated under reduced pressure. The residue was subjected to HPLC separation to give the title compound as yellow solid (32 mg, 19% yield). MS(ESI) m/z 564.2.

### EXAMPLE 21

### Preparation of 1-{4-[7-(2,2-dimethoxyethyl)-4-morpholin-4-yl-7H-pyrrolo[2,3-d]pyrimidin-2-yl]phenyl}-3-pyridin-4-ylurea

### Step 1: Synthesis of 2-chloro-7-(2,2-dimethoxyethyl)-4-morpholin-4-yl-7H-pyrrolo[2,3-d]pyrimidine

To a solution of 2-chloro-4-morpholin-4-yl-7H-pyrrolo[2,3-d]pyrimidine (650 mg, 2.7 mmol) in DMF (10 mL) were added 2-bromo-1,1-dimethoxyethane (0.65 mL, 5.4 mmol) and Cs₂CO₃ (1.067 g, 3.3 mmol). The resulting mixture was heated at 80° C. under nitrogen overnight, and cooled to room temperature. Water was added, and the mixture was extracted with EtOAc. The combined extracts were washed with water and brine, dried over MgSO₄. The solvent was removed under reduced pressure to give the title compound as light yellow solid (665 mg, 75% yield), which was used in next step without further purification. MS(ESI) m/z 327.2.

### Step 2: Synthesis of 4-[7-(2,2-dimethoxyethyl)-4-morpholin-4-yl-7H-pyrrolo[2,3-d]pyrimidin-2-yl]aniline

To a 20 mL vial were added 2-chloro-7-(2,2-dimethoxyethyl)-4-morpholin-4-yl-7H-pyrrolo[2,3-d]pyrimidine (665 mg, 2 mmol), 4-aminophenylboronic acid pinacol ester (670 mg, 3 mmol), Pd(PPh₃)₄ (118 mg, 5 mol %), 1,2-dimethoxyethane (DME, 6 mL) and sodium carbonate aqueous solution (2M, 4 mL). The resulting mixture was heated at 130° C. for 30 min in microwave oven, and then cooled to room temperature. The aqueous phase was extracted with EtOAc, and the combined organic solution was concentrated under reduced pressure. The residue was purified by flash chromatography to give the title compound as brown oil (760 mg, 97% yield). MS(ESI) m/z 384.4.

### Step 3: Synthesis of 1-{4-[7-(2,2-dimethoxyethyl)-4-morpholin-4-yl-7H-pyrrolo[2,3-d]pyrimidin-2-yl]phenyl}-3-pyridin-4-ylurea

To a solution of 4-[7-(2,2-dimethoxyethyl)-4-morpholin-4-yl-7H-pyrrolo[2,3-d]pyrimidin-2-yl]aniline (766 mg, 2 mmol) in CHCl₃ (10 mL) were added Et₃N (0.55 mL, 3.9 mmol) and triphosgene (594 mg, 2 mmol). The mixture was stirred at room temperature for 15 min before a solution of 4-aminopyridine (564 mg, 6 mmol) in THF (10 mL) was added. The mixture was heated at 50° C. overnight. The solvent was removed, and the residue was subjected to HPLC separation to give the title compound as yellow solid (350 mg, 35% yield). MS(ESI) m/z 504.4. HRMS: calcd for C₂₆H₂₉N₇O₄+H⁺, 504.23538; found (ESI, [M+H]⁺), 504.2358.

### EXAMPLE 22

### Preparation of 1-{4-[4-morpholin-4-yl-7-(2-oxoethyl)-7H-pyrrolo[2,3-d]pyrimidin-2-yl]phenyl}-3-pyridin-4-ylurea

A mixture of 1-{4-[7-(2,2-dimethoxyethyl)-4-morpholin-4-yl-7H-pyrrolo[2,3-d]pyrimidin-2-yl]phenyl}-3-pyridin-4-ylurea (300 mg, 0.6 mmol), dioxane (3 mL), and 6M HCl (3 mL) was heated at 70° C. for 3 h, and cooled to room temperature. The mixture was concentrated in vacuum, and the residue was treated with EtOAc. The resulting solid was collected by filtration, and washed with EtOAc to give the title compound as off-white solid (479 mg, 85% yield). MS(ESI) m/z 458.2.

### EXAMPLE 23

### Preparation of 1-{4-[4-morpholin-4-yl-7-(2-pyrrolidin-1-ylethyl)-7H-pyrrolo[2,3-d]pyrimidin-2-yl]phenyl}-3-pyridin-4-ylurea

To a solution of 1-{4-[4-morpholin-4-yl-7-(2-oxoethyl)-7H-pyrrolo[2,3-d]pyrimidin-2-yl]phenyl}-3-pyridin-4-ylurea (24 mg, 0.05 mmol) in MeOH (2 mL) were added pyrrolidine (22 mg, 0.3 mmol), ZnCl₂ (14 mg, 0.1 mmol) and NaBH₃CN (6 mg, 0.1 mmol). The resulting mixture was stirred at room temperature for 2 h, and 0.5 mL of NaOH (1M in water) was added. The solvent was removed, and the residue was subjected to HPLC separation to give the title compound as off-white solid (9.2 mg, 25% yield). MS(ESI) m/z 513.5.

### EXAMPLE 24

### Preparation of 1-{4-[4-morpholin-4-yl-7-(2-piperidin-1-ylethyl)-7H-pyrrolo[2,3-d]pyrimidin-2-yl]phenyl}-3-pyridin-4-ylurea

Following the procedure described as in Example 23, reductive amination of 1-{4-[4-morpholin-4-yl-7-(2-oxoethyl)-7H-pyrrolo[2,3-d]pyrimidin-2-yl]phenyl}-3-pyridin-4-ylurea (24 mg, 0.05 mmol) and piperidine (26 mg, 0.3 mmol) yielded the title compound as off-white solid (10.2 mg, 27% yield). MS(ESI) m/z 527.5.

### EXAMPLE 25

### Preparation of 1-[4-(7-{2-[(4-fluorophenyl)amino]ethyl}-4-morpholin-4-yl-7H-pyrrolo[2,3-d]pyrimidin-2-yl)phenyl]-3-pyridin-4-ylurea

Following the procedure described as in Example 23, reductive amination of 1-{4-[4-morpholin-4-yl-7-(2-oxoethyl)-7H-pyrrolo[2,3-d]pyrimidin-2-yl]phenyl}-3-pyridin-4-ylurea (24 mg, 0.05 mmol) and 4-fluoroaniline (33 mg, 0.3 mmol) yielded the title compound as off-white solid (8.8 mg, 23% yield). MS(ESI) m/z 553.5.

### EXAMPLE 26

### Preparation of 1-[4-(4-morpholin-4-yl-7-{2-[(pyridin-3-ylmethyl)amino]ethyl}-7H-pyrrolo[2,3-d]pyrimidin-2-yl)phenyl]-3-pyridin-4-ylurea

Following the procedure described as in Example 23, reductive amination of 1-{4-[4-morpholin-4-yl-7-(2-oxoethyl)-7H-pyrrolo[2,3-d]pyrimidin-2-yl]phenyl}-3-pyridin-4-ylurea (24 mg, 0.05 mmol) and 4-(aminomethyl)pyridine (32 mg, 0.3 mmol) yielded the title compound as off-white solid (17.2 mg, 44% yield). MS(ESI) m/z 550.3.

### EXAMPLE 27

### Preparation of 1-{4-[7-(2-{[2-(dimethylamino)ethyl]amino}ethyl)-4-morpholin-4-yl-7H-pyrrolo[2,3-d]pyrimidin-2-yl]phenyl}-3-pyridin-4-ylurea

Following the procedure described as in Example 23, reductive amination of 1-{4-[4-morpholin-4-yl-7-(2-oxoethyl)-7H-pyrrolo[2,3-d]pyrimidin-2-yl]phenyl}-3-pyridin-4-ylurea (24 mg, 0.05 mmol) and N,N-dimethylethylenediamine (26 mg, 0.3 mmol) yielded the title compound as off-white solid (16 mg, 37% yield). MS(ESI) m/z 530.3.

### EXAMPLE 28

### Preparation of 1-(4-{7-[2-(4-methylpiperazin-1-yl)ethyl]-4-morpholin-4-yl-7H-pyrrolo[2,3-d]pyrimidin-2-yl}phenyl)-3-pyridin-4-ylurea

Following the procedure described as in Example 23, reductive amination of 1-{4-[4-morpholin-4-yl-7-(2-oxoethyl)-7H-pyrrolo[2,3-d]pyrimidin-2-yl]phenyl}-3-pyridin-4-ylurea (24 mg, 0.05 mmol) and 1-methylpiperazine (30 mg, 0.3 mmol) yielded the title compound as off-white solid (18.6 mg, 42% yield). MS(ESI) m/z 542.3.

### EXAMPLE 29

### Preparation of 1-{4-[4-morpholin-4-yl-7-(2-piperazin-1-ylethyl)-7H-pyrrolo[2,3-d]pyrimidin-2-yl]phenyl}-3-pyridin-4-ylurea

Following the procedure described as in Example 23, reductive amination of 1-{4-[4-morpholin-4-yl-7-(2-oxoethyl)-7H-pyrrolo[2,3-d]pyrimidin-2-yl]phenyl}-3-pyridin-4-ylurea (24 mg, 0.05 mmol) and piperazine (26 mg, 0.3 mmol) yielded the title compound as off-white solid (17 mg, 39% yield). MS(ESI) m/z 528.3.

### EXAMPLE 30

### Preparation of 1-{4-[7-(2-{[2-(1H-imidazol-5-yl)ethyl]amino}ethyl)-4-morpholin-4-yl-7H-pyrrolo[2,3-d]pyrimidin-2-yl]phenyl}-3-pyridin-4-ylurea

Following the procedure described as in Example 23, reductive amination of 1-{4-[4-morpholin-4-yl-7-(2-oxoethyl)-7H-pyrrolo[2,3-d]pyrimidin-2-yl]phenyl}-3-pyridin-4-ylurea (24 mg, 0.05 mmol) and histamine base (33 mg, 0.3 mmol) yielded the title compound as off-white solid (7 mg, 16% yield). MS(ESI) m/z 553.2.

### EXAMPLE 31

### Preparation of 1-(4-{7-[2-(tert-butylamino)ethyl]-4-morpholin-4-yl-7H-pyrrolo[2,3-d]pyrimidin-2-yl}phenyl)-3-pyridin-4-ylurea

Following the procedure described as in Example 23, reductive amination of 1-{4-[4-morpholin-4-yl-7-(2-oxoethyl)-7H-pyrrolo[2,3-d]pyrimidin-2-yl]phenyl}-3-pyridin-4-ylurea (24 mg, 0.05 mmol) and tert-butylamine (22 mg, 0.3 mmol) yielded the title compound as off-white solid (8.6 mg, 23% yield). MS(ESI) m/z 515.3.

### EXAMPLE 32

### Preparation of 1-(4-{7-[2-(isopropylamino)ethyl]-4-morpholin-4-yl-7H-pyrrolo[2,3-d]pyrimidin-2-yl}phenyl)-3-pyridin-4-ylurea

Following the procedure described as in Example 23, reductive amination of 1-{4-[4-morpholin-4-yl-7-(2-oxoethyl)-7H-pyrrolo[2,3-d]pyrimidin-2-yl]phenyl}-3-pyridin-4-ylurea (24 mg, 0.05 mmol) and isopropylamine (18 mg, 0.3 mmol) yielded the title compound as off-white solid (11.3 mg, 31% yield). MS(ESI) m/z 501.5.

### EXAMPLE 33

### Preparation of 1-(4-{7-[2-(methylamino)ethyl]-4-morpholin-4-yl-7H-pyrrolo[2,3-d]pyrimidin-2-yl}phenyl)-3-pyridin-4-ylurea

Following the procedure described as in Example 23, reductive amination of 1-{4-[4-morpholin-4-yl-7-(2-oxoethyl)-7H-pyrrolo[2,3-d]pyrimidin-2-yl]phenyl}-3-pyridin-4-ylurea (24 mg, 0.05 mmol) and methylamine (2M in THF, 0.15 mL, 0.3 mmol) yielded the title compound as off-white solid (17.1 mg, 49% yield). MS(ESI) m/z 473.5.

### EXAMPLE 34

### Preparation of 1-{4-[7-(2-hydroxyethyl)-4-morpholin-4-yl-7H-pyrrolo[2,3-d]pyrimidin-2-yl]phenyl}-3-pyridin-4-ylurea

To a stirred mixture of 1-{4-[4-morpholin-4-yl-7-(2-oxoethyl)-7H-pyrrolo[2,3-d]pyrimidin-2-yl]phenyl}-3-pyridin-4-ylurea (215 mg, 0.47 mmol), MeOH (4 mL) and THF (4 mL) was added NaBH₄ (27 mg, 0.7 mmol). The resulting mixture was stirred at room temperature for 30 min, and 2 mL of NaOH (1M in water) was added. The mixture was concentrated in vacuum, and the residue was subjected to HPLC separation to give the title compound as off-white solid (165 mg, 76% yield). MS(ESI) m/z 460.5. HRMS: calcd for C₂₄H₂₅N₇O₃+H⁺, 460.20916; found (ESI, [M+H]⁺ Calc'd), 460.2092.

### EXAMPLE 35

### Preparation of 1-(4-{7-[(2,5-dioxoimidazolidin-4-yl)methyl]-4-morpholin-4-yl-7H-pyrrolo[2,3-d]pyrimidin-2-yl}phenyl)-3-pyridin-4-ylurea

To a stirred mixture of 1-{4-[4-morpholin-4-yl-7-(2-oxoethyl)-7H-pyrrolo[2,3-d]pyrimidin-2-yl]phenyl}-3-pyridin-4-ylurea (70 mg, 0.15 mmol), EtOH (2 mL) and H₂O (2 mL) were added KCN (11 mg, 0.16 mmol) and (NH₄)₂CO₃ (43 mg, 0.45 mmol). The resulting mixture was heated at 60° C. overnight. The mixture was concentrated in vacuum, and the residue was subjected to HPLC separation to give the title compound as yellow solid (43 mg, 53% yield). MS(ESI) m/z 528.5.

### EXAMPLE 36

### Preparation of 1-{4-[4-morpholin-4-yl-7-(2,2,2-trifluoroethyl)-7H-pyrrolo[2,3-d]pyrimidin-2-yl]phenyl}-3-pyridin-4-ylurea

### Step 1: Synthesis of 2-chloro-4-morpholin-4-yl-7-(2,2,2-trifluoroethyl)-7H-pyrrolo[2,3-d]pyrimidine

To a solution of 2-chloro-4-morpholin-4-yl-7H-pyrrolo[2,3-d]pyrimidine (340 mg, 1.4 mmol) in DMF (5 mL) were added 1,1,1-trifluoro-2-iodoethane (0.28 mL, 2.8 mmol) and Cs₂CO₃ (559 mg, 1.7 mmol). The resulting mixture was heated at 80° C. under nitrogen overnight, and cooled to room temperature. The reaction mixture was quenched with water and extracted EtOAc. The combined extracts were washed with water and brine, dried over MgSO₄. The solvent was removed under reduced pressure to give the title compound as light yellow solid (199 mg, 43% yield), which was used in next step without further purification. MS(ESI) m/z 321.3.

### Step 2: Synthesis of 4-[7-(2,2,2-trifluoroethyl)-4-morpholin-4-yl-7H-pyrrolo[2,3-d]pyrimidin-2-yl]aniline

To a 10 mL vial were added 2-chloro-4-morpholin-4-yl-7-(2,2,2-trifluoroethyl)-7H-pyrrolo[2,3-d]pyrimidine (294 mg, 0.9 mmol), 4-aminophenylboronic acid pinacol ester (302 mg, 1.4 mmol), Pd(PPh₃)₄ (53 mg, 5 mol %), 1,2-dimethoxyethane (DME, 3 mL) and sodium carbonate aqueous solution (2M, 2 mL). The resulting mixture was heated at 130° C. for 30 min in microwave oven, and then cooled to room temperature. The aqueous phase was extracted with EtOAc, and the combined organic solution was concentrated under reduced pressure. The residue was purified by flash chromatography to give the title compound as brown oil (286 mg, 83% yield). MS(ESI) m/z 378.4.

### Step 3: Synthesis of 1-{4-[7-(2,2,2-trifluoroethyl)-4-morpholin-4-yl-7H-pyrrolo[2,3-d]pyrimidin-2-yl]phenyl}-3-pyridin-4-ylurea

To a solution of 4-[7-(2,2,2-trifluoroethyl)-4-morpholin-4-yl-7H-pyrrolo[2,3-d]pyrimidin-2-yl]aniline (25 mg, 0.066 mmol) in CHCl₃ (1 mL) were added Et₃N (28 μL, 0.2 mmol) and triphosgene (20 mg, 0.066 mmol). The mixture was stirred at room temperature for 15 min before a solution of 4-aminopyridine (19 mg, 0.2 mmol) in THF (1 mL) was added. The mixture was stirred at room temperature overnight. The solvent was removed, and the residue was subjected to HPLC separation to give the title compound as off-white solid (24.5 mg, 61% yield). MS(ESI) m/z 498.4.

### EXAMPLE 37

### Preparation of 1-{4-[4-morpholin-4-yl-7-(2,2,2-trifluoroethyl)-7H-pyrrolo[2,3-d]pyrimidin-2-yl]phenyl}-3-pyridin-3-ylurea

Following the procedure described in Example 36, reaction of 4-[7-(2,2,2-trifluoroethyl)-4-morpholin-4-yl-7H-pyrrolo[2,3-d]pyrimidin-2-yl]aniline (25 mg, 0.066 mmol) and triphosgene (20 mg, 0.066 mmol) and 3-aminopyridine (19 mg, 0.2 mmol) gave the title compound as off-white solid (28.4 mg, 70% yield). MS(ESI) m/z 498.4.

### EXAMPLE 38

### Preparation of 1-(4-fluorophenyl)-3-{4-[4-morpholin-4-yl-7-(2,2,2-trifluoroethyl)-7H-pyrrolo[2,3-d]pyrimidin-2-yl]phenyl}urea

Following the procedure described in Example 36, reaction of 4-[7-(2,2,2-trifluoroethyl)-4-morpholin-4-yl-7H-pyrrolo[2,3-d]pyrimidin-2-yl]aniline (25 mg, 0.066 mmol) and triphosgene (20 mg, 0.066 mmol) and 4-fluoroaniline (22 mg, 0.2 mmol) gave the title compound as off-white solid (22.6 mg, 67% yield). MS(ESI) m/z 515.4.

### EXAMPLE 39

### Preparation of 1-[4-(4-methylpiperazin-1-yl)phenyl]-3-{4-[4-morpholin-4-yl-7-(2,2,2-trifluoroethyl)-7H-pyrrolo[2,3-d]pyrimidin-2-yl]phenyl}urea

Following the procedure described in Example 36, reaction of 4-[7-(2,2,2-trifluoroethyl)-4-morpholin-4-yl-7H-pyrrolo[2,3-d]pyrimidin-2-yl]aniline (25 mg, 0.066 mmol) and triphosgene (20 mg, 0.066 mmol) and 4-(4-methylpiperazino)aniline (38 mg, 0.2 mmol) gave the title compound as off-white solid (37 mg, 68% yield). MS(ESI) m/z 595.3.

### EXAMPLE 40

### Preparation of 1-[4-(hydroxymethyl)phenyl]-3-{4-[4-morpholin-4-yl-7-(2,2,2-trifluoroethyl)-7H-pyrrolo[2,3-d]pyrimidin-2-yl]phenyl}urea

Following the procedure described in Example 36, reaction of 4-[7-(2,2,2-trifluoroethyl)-4-morpholin-4-yl-7H-pyrrolo[2,3-d]pyrimidin-2-yl]aniline (25 mg, 0.066 mmol) and triphosgene (20 mg, 0.066 mmol) and 4-aminobenzylalcohol (25 mg, 0.2 mmol) gave the title compound as off-white solid (23.5 mg, 68% yield). MS(ESI) m/z 527.2.

### EXAMPLE 41

### Preparation of 1-[2-(dimethylamino)ethyl]-3-{4-[4-morpholin-4-yl-7-(2,2,2-trifluoroethyl)-7H-pyrrolo[2,3-d]pyrimidin-2-yl]phenyl}urea

Following the procedure described in Example 36, reaction of 4-[7-(2,2,2-trifluoroethyl)-4-morpholin-4-yl-7H-pyrrolo[2,3-d]pyrimidin-2-yl]aniline (25 mg, 0.066 mmol) and triphosgene (20 mg, 0.066 mmol) and N,N-dimethylethylenediamine (18 mg, 0.2 mmol) gave the title compound as off-white solid (27.2 mg, 68% yield). MS(ESI) m/z 492.2.

### EXAMPLE 42

### Preparation of 1-(2-hydroxyethyl)-3-{4-[4-morpholin-4-yl-7-(2,2,2-trifluoroethyl)-7H-pyrrolo[2,3-d]pyrimidin-2-yl]phenyl}urea

Following the procedure described in Example 36, reaction of 4-[7-(2,2,2-trifluoroethyl)-4-morpholin-4-yl-7H-pyrrolo[2,3-d]pyrimidin-2-yl]aniline (25 mg, 0.066 mmol) and triphosgene (20 mg, 0.066 mmol) and ethanolamine (13 mg, 0.2 mmol) gave the title compound as off-white solid (23.2 mg, 76% yield). MS(ESI) m/z 465.2.

### EXAMPLE 43

### Preparation of 2-hydroxyethyl{4-[4-morpholin-4-yl-7-(2,2,2-trifluoroethyl)-7H-pyrrolo[2,3-d]pyrimidin-2-yl]phenyl}carbamate

Following the procedure described in Example 36, reaction of 4-[7-(2,2,2-trifluoroethyl)-4-morpholin-4-yl-7H-pyrrolo[2,3-d]pyrimidin-2-yl]aniline (25 mg, 0.066 mmol) and triphosgene (20 mg, 0.066 mmol) and ethylene glycol (13 mg, 0.2 mmol) gave the title compound as off-white solid (18.4 mg, 60% yield). MS(ESI) m/z 466.1.

### EXAMPLE 44

### Preparation of 1-[4-(7-methyl-4-morpholin-4-yl-7H-pyrrolo[2,3-d]pyrimidin-2-yl)phenyl]-3-pyridin-3-ylurea

Following the procedure described in Example 36, reaction of 4-(7-methyl-4-morpholin-4-yl-7H-pyrrolo[2,3-d]pyrimidin-2-yl)aniline (20 mg, 0.066 mmol) and triphosgene (20 mg, 0.066 mmol) and 3-aminopyridine (19 mg, 0.2 mmol) gave the title compound as off-white solid (9.4 mg, 26% yield). MS(ESI) m/z 430.4.

### EXAMPLE 45

### Preparation of 5-[4-morpholin-4-yl-7-(2,2,2-trifluoroethyl)-7H-pyrrolo[2,3-d]pyrimidin-2-yl]-1H-benzimidazol-2-amine

To a 10 mL vial were added 2-chloro-4-morpholin-4-yl-7-(2,2,2-trifluoroethyl)-7H-pyrrolo[2,3-d]pyrimidine (222 mg, 0.7 mmol), 5-(4,4,5,5-tetramethyl-1,3,2-dioxaborolan-2-yl)-1H-benzimidazol-2-amine (216 mg, 0.83 mmol), Pd(PPh₃)₄ (40 mg, 5 mol %), DMF (4 mL) and potassium bicarbonate aqueous solution (2M, 1.5 mL). The resulting mixture was heated at 180° C. for 10 min in microwave oven, and then cooled to room temperature. The reaction mixture was quenched with water and extracted with EtOAc. The combined organic solution was concentrated under reduced pressure and the residue was subjected to HPLC separation to give the title compound as off-white solid (97 mg, 34% yield). MS(ESI) m/z 418.1. HRMS: calcd for C₁₉H₁₈F₃N₇O+H⁺, 418.15977; found (ESI, [M+H]⁺ Calc'd), 418.1598.

### EXAMPLE 46

### Preparation of 1-{5-[4-morpholin-4-yl-7-(2,2,2-trifluoroethyl)-7H-pyrrolo[2,3-d]pyrimidin-2-yl]-1H-benzimidazol-2-yl}-3-pyridin-3-ylurea

A mixture of 5-[4-morpholin-4-yl-7-(2,2,2-trifluoroethyl)-7H-pyrrolo[2,3-d]pyrimidin-2-yl]-1H-benzimidazol-2-amine (80 mg, 0.19 mmol), THF (3 mL), CHCl₃ (3 mL), Et₃N (0.05 mL, 0.38 mmol), and 3-isocyanatopyridine (46 mg, 0.38 mmol) was stirred at room temperature overnight. The solvent was removed, and the residue was subjected to HPLC separation to give the title compound as off-white solid (68 mg, 66% yield). MS(ESI) m/z 538.4.

### EXAMPLE 47

### Preparation of N-{5-[4-morpholin-4-yl-7-(2,2,2-trifluoroethyl)-7H-pyrrolo[2,3-d]pyrimidin-2-yl]-1H-benzimidazol-2-yl}isonicotinamide

To a solution of 5-[4-morpholin-4-yl-7-(2,2,2-trifluoroethyl)-7H-pyrrolo[2,3-d]pyrimidin-2-yl]-1H-benzimidazol-2-amine (20 mg, 0.05 mmol) in DMF (3 mL) were added Et₃N (20 μL, 0.15 mmol), isonicotinic acid (9 mg, 0.07 mmol) and O-(Benzotrizol-1-yl)-N—N—N,N-tetramethyluronium hexafluorophosphate (HBTU, 55 mg, 0.15 mmol). The mixture was stirred at room temperature overnight. The solvent was removed, and the residue was subjected to HPLC separation to give the title compound as yellow solid (8 mg, 32% yield). MS(ESI) m/z 523.4.

### EXAMPLE 48

### Preparation of N-methyl-5-[4-morpholin-4-yl-7-(2,2,2-trifluoroethyl)-7H-pyrrolo[2,3-d]pyrimidin-2-yl]-1H-benzimidazol-2-amine

A mixture of 5-[4-morpholin-4-yl-7-(2,2,2-trifluoroethyl)-7H-pyrrolo[2,3-d]pyrimidin-2-yl]-1H-benzimidazol-2-amine (30 mg, 0.07 mmol), acetone (3 mL), K₂CO₃ (29 mg, 0.2 mmol), and iodomethane (14 mg, 0.1 mmol). The mixture was refluxed overnight. The solvent was removed, and the residue was subjected to HPLC separation to give the title compound as off-white solid (9 mg, 29% yield). MS(ESI) m/z 432.4.

### EXAMPLE 49

### Preparation of ethyl {5-[4-morpholin-4-yl-7-(2,2,2-trifluoroethyl)-7H-pyrrolo[2,3-d]pyrimidin-2-yl]-1H-benzimidazol-2-yl}carbamate

A mixture of 5-[4-morpholin-4-yl-7-(2,2,2-trifluoroethyl)-7H-pyrrolo[2,3-d]pyrimidin-2-yl]-1H-benzimidazol-2-amine (20 mg, 0.05 mmol), CHCl₃ (2 mL), Et₃N (0.02 mL, 0.15 mmol), and ethyl chloroformate (8 mg, 0.07 mmol) was stirred at room temperature for 3 hours. The solvent was removed, and the residue was subjected to HPLC separation to give the title compound as off-white solid (20 mg, 85% yield). MS(ESI) m/z 490.4.

### EXAMPLE 50

### Preparation of methyl 4-[({4-[4-morpholin-4-yl-7-(2,2,2-trifluoroethyl)-7H-pyrrolo[2,3-d]pyrimidin-2-yl]phenyl}carbamoyl)amino]benzoate

To a solution of 4-[7-(2,2,2-trifluoroethyl)-4-morpholin-4-yl-7H-pyrrolo[2,3-d]pyrimidin-2-yl]aniline (479 mg, 1.3 mmol) in CH₂Cl₂ (10 mL) was added methyl 4-isocyatobenzoate (269 mg, 1.5 mmol), and the resulting mixture was stirred at room temperature overnight. The resulting solid was collected by filtration and washed with CH₂Cl₂ to give the product as off-white solid (539 mg, 77% yield). MS(ESI) m/z 555.4.

### EXAMPLE 51

### Preparation of N-[2-(dimethylamino)ethyl]-N-methyl-4-[({4-[4-morpholin-4-yl-7-(2,2,2-trifluoroethyl)-7H-pyrrolo[2,3-d]pyrimidin-2-yl]phenyl}carbamoyl)amino]benzamide

### Step 1: Synthesis of 4-({[4-(7-(2,2,2-trifluoroethyl)-4-morpholin-4-yl-7H-pyrrolo[2,3-d]pyrimidin-2-yl)phenyl]carbamoyl}amino)benzoic Acid

To a solution of methyl 4-[({4-[4-morpholin-4-yl-7-(2,2,2-trifluoroethyl)-7H-pyrrolo[2,3-d]pyrimidin-2-yl]phenyl}carbamoyl)amino]benzoate (500 mg, 0.9 mmol) in MeOH (30 mL) and THF (10 mL) was added 1N NaOH aqueous solution (2.7 mL), and the mixture was heated at 70° C. overnight. The mixture was cooled to room temperature, and concentrated in vacuo. The residue was treated water, and acidified to pH 4-5 by addition of 1N HCl, and the resulting solid was collected by filtration, and washed with water and dried to give the product as off-white solid (486 mg, 100% yield). MS(ESI) m/z 541.4.

### Step 2: Synthesis of N-[2-(dimethylamino)ethyl]-N-methyl-4-[({4-[4-morpholin-4-yl-7-(2,2,2-trifluoroethyl)-7H-pyrrolo[2,3-d]pyrimidin-2-yl]phenyl}carbamoyl)amino]benzamide

To a solution of 4-({[4-(7-(2,2,2-trifluoroethyl)-4-morpholin-4-yl-7H-pyrrolo[2,3-d]pyrimidin-2-yl)phenyl]carbamoyl}amino)benzoic acid (32 mg, 0.06 mmol) in THF (2 mL) were added N,N,N′-trimethylethylenediamine (12 mg, 0.12 mmol), Et3N (12 mg, 0.12 mmol), HOBT (16 mg, 12 mmol) and EDCI (23 mg, 0.12 mmol). The resulting mixture was stirred at room temperature overnight, and concentrated in vacuo. The residue was subjected to HPLC separation to give the product as off-white solid (1TFA salt, 38.6 mg, 87% yield). MS(ESI) m/z 625.5. HRMS: calcd for C₃₁H₃₅F₃N₈O₃+H⁺, 625.28570; found (ESI, [M+H]⁺ Calc'd), 625.2857.

### EXAMPLE 52

### Preparation of N-[2-(dimethylamino)ethyl]-4-[({4-[4-morpholin-4-yl-7-(2,2,2-trifluoroethyl)-7H-pyrrolo[2,3-d]pyrimidin-2-yl]phenyl}carbamoyl)amino]benzamide

Following the procedure described in Example 51, reaction of 4-({[4-(7-(2,2,2-trifluoroethyl)-4-morpholin-4-yl-7H-pyrrolo[2,3-d]pyrimidin-2-yl)phenyl]carbamoyl}amino)benzoic acid (32 mg, 0.06 mmol) and N,N-dimethylethylenediamine (11 mg, 0.12 mmol) gave the title compound as off-white solid (1TFA salt, 42.9 mg, 99% yield). MS(ESI) m/z 611.5. HRMS: calcd for C₃₀H₃₃F₃N₈O₃+H⁺, 611.27005; found (ESI, [M+H]⁺ Calc'd), 611.2700.

### EXAMPLE 53

### Preparation of N-methyl-N-[2-(methylamino)ethyl]-4-[({4-[4-morpholin-4-yl-7-(2,2,2-trifluoroethyl)-7H-pyrrolo[2,3-d]pyrimidin-2-yl]phenyl}carbamoyl)amino]benzamide

Following the procedure described in Example 51, reaction of 4-({[4-(7-(2,2,2-trifluoroethyl)-4-morpholin-4-yl-7H-pyrrolo[2,3-d]pyrimidin-2-yl)phenyl]carbamoyl}amino)benzoic acid (32 mg, 0.06 mmol) and N,N′-dimethylethylenediamine (11 mg, 0.12 mmol) gave the title compound as off-white solid (1TFA salt, 11 mg, 25% yield). MS(ESI) m/z 611.5.

### EXAMPLE 54

### Preparation of 1-{4-[(4-methylpiperazin-1-yl)carbonyl]phenyl}-3-{4-[4-morpholin-4-yl-7-(2,2,2-trifluoroethyl)-7H-pyrrolo[2,3-d]pyrimidin-2-yl]phenyl}urea

Following the procedure described in Example 51, reaction of 4-({[4-(7-(2,2,2-trifluoroethyl)-4-morpholin-4-yl-7H-pyrrolo[2,3-d]pyrimidin-2-yl)phenyl]carbamoyl}amino)benzoic acid (32 mg, 0.06 mmol) and 1-methylpiperazine (12 mg, 0.12 mmol) gave the title compound as off-white solid (1TFA salt, 43 mg, 97% yield). MS(ESI) m/z 623.2. HRMS: calcd for C₃₁H₃₃F₃N₈O₃+H⁺, 623.27005; found (ESI, [M+H]⁺ Calc'd), 623.2700.

### EXAMPLE 55

### Preparation of 1-{4-[(3,3-dimethylpiperazin-1-yl)carbonyl]phenyl}-3-{4-[4-morpholin-4-yl-7-(2,2,2-trifluoroethyl)-7H-pyrrolo[2,3-d]pyrimidin-2-yl]phenyl}urea

Following the procedure described in Example 51, reaction of 4-({[4-(7-(2,2,2-trifluoroethyl)-4-morpholin-4-yl-7H-pyrrolo[2,3-d]pyrimidin-2-yl)phenyl]carbamoyl}amino)benzoic acid (32 mg, 0.06 mmol) and 2,2-dimethylpiperazine (14 mg, 0.12 mmol) gave the title compound as off-white solid (1TFA salt, 21.3 mg, 47% yield). MS(ESI) m/z 637.2.

### EXAMPLE 56

### Preparation of 4-[({4-[4-morpholin-4-yl-7-(2,2,2-trifluoroethyl)-7H-pyrrolo[2,3-d]pyrimidin-2-yl]phenyl}carbamoyl)amino]-N-(2-piperidin-1-ylethyl)benzamide

Following the procedure described in Example 51, reaction of 4-({[4-(7-(2,2,2-trifluoroethyl)-4-morpholin-4-yl-7H-pyrrolo[2,3-d]pyrimidin-2-yl)phenyl]carbamoyl}amino)benzoic acid (32 mg, 0.06 mmol) and 1-(2-aminoethyl)piperidine (15 mg, 0.12 mmol) gave the title compound as off-white solid (1TFA salt, 45 mg, 98% yield). MS(ESI) m/z 651.2. HRMS: calcd for C₃₃H₃₇F₃N₈O₃+H⁺, 651.30135; found (ESI, [M+H]⁺ Calc'd), 651.3013.

### EXAMPLE 57

### Preparation of 1-(4-{[4-(dimethylamino)piperidin-1-yl]carbonyl}phenyl)-3-{4-[4-morpholin-4-yl-7-(2,2,2-trifluoroethyl)-7H-pyrrolo[2,3-d]pyrimidin-2-yl]phenyl}urea

Following the procedure described in Example 51, reaction of 4-({[4-(7-(2,2,2-trifluoroethyl)-4-morpholin-4-yl-7H-pyrrolo[2,3-d]pyrimidin-2-yl)phenyl]carbamoyl}amino)benzoic acid (150 mg, 0.28 mmol) and 4-dimethylaminopiperidine (71 mg, 0.56 mmol) gave the title compound as off-white solid (1HCl salt, 130 mg, 68% yield). MS(ESI) m/z 651.4. HRMS: calcd for C₃₃H₃₇F₃N₈O₃+H⁺, 651.30135; found (ESI, [M+H]⁺ Calc'd), 651.3013.

### EXAMPLE 58

### Preparation of 1-{4-[2-(dimethylamino)ethoxy]phenyl}-3-{4-[4-morpholin-4-yl-7-(2,2,2-trifluoroethyl)-7H-pyrrolo[2,3-d]pyrimidin-2-yl]phenyl}urea

To a solution of 4-[7-(2,2,2-trifluoroethyl)-4-morpholin-4-yl-7H-pyrrolo[2,3-d]pyrimidin-2-yl]aniline (155 mg, 0.41 mmol) in CHCl₃ (5 mL) were added Et₃N (0.17 mL, 1.2 mmol) and triphosgene (73 mg, 0.24 mmol). The mixture was stirred at room temperature for 15 min, and 4-(2-dimethylamino)ethoxy)aniline hydrochloride (308 mg, 1.23 mmol) was added. The mixture was stirred at room temperature overnight. The solvent was removed, and the residue was subjected to HPLC separation to give the title compound as off-white solid (75 mg, 29% yield). MS(ESI) m/z 584.4, HRMS: calcd for C₂₉H₃₂F₃N₇O₃+H⁺, 584.25915; found (ESI-FTMS, [M+H]⁺), 584.26031.

### EXAMPLE 59

### Preparation of methyl 4-({[4-(7-ethyl-4-morpholin-4-yl-7H-pyrrolo[2,3-d]pyrimidin-2-yl)phenyl]carbamoyl}amino)benzoate

To a solution of 4-(7-ethyl-4-morpholin-4-yl-7H-pyrrolo[2,3-d]pyrimidin-2-yl)aniline (1.72 g, 5.3 mmol) in CH₂Cl₂ (50 mL) was added methyl 4-isocyatobenzoate (1.13 g, 6.4 mmol), and the resulting mixture was stirred at room temperature overnight. The resulting solid was collected by filtration and washed with CH₂Cl₂ to give the product as off-white solid (1.81 g, 68% yield). MS(ESI) m/z 501.4.

### EXAMPLE 60

### Preparation of 4-({[4-(7-ethyl-4-morpholin-4-yl-7H-pyrrolo[2,3-d]pyrimidin-2-yl)phenyl]carbamoyl}amino)benzoic acid

To a solution of methyl 4-({[4-(7-ethyl-4-morpholin-4-yl-7H-pyrrolo[2,3-d]pyrimidin-2-yl)phenyl]carbamoyl}amino)benzoate (1.81 g, 3.6 mmol) in MeOH (50 mL) and THF (20 mL) was added 1N NaOH aqueous solution (18 mL), and the mixture was heated at 70° C. for 3 hours. The mixture was cooled to room temperature, and concentrated in vacuo. The residue was treated water, and acidified to pH 4-5 by addition of 1N HCl, and the resulting solid was collected by filtration, and washed with water and dried to give the product as off-white solid (1.65 g, 94% yield). MS(ESI) m/z 487.5.

### EXAMPLE 61

### Preparation of N-[2-(dimethylamino)ethyl]-4-({[4-(7-ethyl-4-morpholin-4-yl-7H-pyrrolo[2,3-d]pyrimidin-2-yl)phenyl]carbamoyl}amino)-N-methylbenzamide

To a solution of 4-({[4-(7-ethyl-4-morpholin-4-yl-7H-pyrrolo[2,3-d]pyrimidin-2-yl)phenyl]carbamoyl}amino)benzoic acid (29 mg, 0.06 mmol) in THF (2 mL) were added N,N,N′-trimethylethylenediamine (12 mg, 0.12 mmol), Et₃N (12 mg, 0.12 mmol), HOBT (16 mg, 12 mmol) and EDCI (23 mg, 0.12 mmol). The resulting mixture was stirred at room temperature overnight, and concentrated in vacuo. The residue was subjected to HPLC separation to give the product as off-white solid (20.8 mg, 61% yield). MS(ESI) m/z 571.4.

### EXAMPLE 62

### Preparation of N-[2-(dimethylamino)ethyl]-4-({[4-(7-ethyl-4-morpholin-4-yl-7H-pyrrolo[2,3-d]pyrimidin-2-yl)phenyl]carbamoyl}amino)benzamide

Following the procedure described in Example 61, reaction of 4-({[4-(7-ethyl-4-morpholin-4-yl-7H-pyrrolo[2,3-d]pyrimidin-2-yl)phenyl]carbamoyl}amino)benzoic acid (29 mg, 0.06 mmol) and N,N-dimethylethylenediamine (11 mg, 0.12 mmol) gave the title compound as off-white solid (17.9 mg, 54% yield). MS(ESI) m/z 557.4.

### EXAMPLE 63

### Preparation of 1-[4-(7-ethyl-4-morpholin-4-yl-7H-pyrrolo[2,3-d]pyrimidin-2-yl)phenyl]-3-{4-[(4-methylpiperazin-1-yl)carbonyl]phenyl}urea

Following the procedure described in Example 61, reaction of 4-({[4-(7-ethyl-4-morpholin-4-yl-7H-pyrrolo[2,3-d]pyrimidin-2-yl)phenyl]carbamoyl}amino)benzoic acid (29 mg, 0.06 mmol) and 1-methylpiperazine (12 mg, 0.12 mmol) gave the title compound as off-white solid (12 mg, 35% yield). MS(ESI) m/z 569.4.

### EXAMPLE 64

### Preparation of 1-(4-{[(3R,5S)-3,5-dimethylpiperazin-1-yl]carbonyl}phenyl)-3-[4-(7-ethyl-4-morpholin-4-yl-7H-pyrrolo[2,3-d]pyrimidin-2-yl)phenyl]urea

Following the procedure described in Example 61, reaction of 4-({[4-(7-ethyl-4-morpholin-4-yl-7H-pyrrolo[2,3-d]pyrimidin-2-yl)phenyl]carbamoyl}amino)benzoic acid (29 mg, 0.06 mmol) and cis-2,6-dimethylpiperazine (14 mg, 0.12 mmol) gave the title compound as off-white solid (21.3 mg, 61% yield). MS(ESI) m/z 583.4.

### EXAMPLE 65

### Preparation of 1-(4-{[4-(dimethylamino)piperidin-1-yl]carbonyl}phenyl)-3-[4-(7-ethyl-4-morpholin-4-yl-7H-pyrrolo[2,3-d]pyrimidin-2-yl)phenyl]urea

Following the procedure described in Example 61, reaction of 4-({[4-(7-ethyl-4-morpholin-4-yl-7H-pyrrolo[2,3-d]pyrimidin-2-yl)phenyl]carbamoyl}amino)benzoic acid (29 mg, 0.06 mmol) and 4-dimethylaminopiperidine (15 mg, 0.12 mmol) gave the title compound as off-white solid (25 mg, 70% yield). MS(ESI) m/z 597.4.

### EXAMPLE 66

### Preparation of 1-[4-(7-ethyl-4-morpholin-4-yl-7H-pyrrolo[2,3-d]pyrimidin-2-yl)phenyl]-3-[4-(morpholin-4-ylcarbonyl)phenyl]urea

Following the procedure described in Example 61, reaction of 4-({[4-(7-ethyl-4-morpholin-4-yl-7H-pyrrolo[2,3-d]pyrimidin-2-yl)phenyl]carbamoyl}amino)benzoic acid (29 mg, 0.06 mmol) and morpholine (11 mg, 0.12 mmol) gave the title compound as off-white solid (21.2 mg, 64% yield). MS(ESI) m/z 556.3.

### EXAMPLE 67

### Preparation of 1-[4-(7-ethyl-4-morpholin-4-yl-7H-pyrrolo[2,3-d]pyrimidin-2-yl)phenyl]-3-[4-(piperazin-1-ylcarbonyl)phenyl]urea

Following the procedure described in Example 61, reaction of 4-({[4-(7-ethyl-4-morpholin-4-yl-7H-pyrrolo[2,3-d]pyrimidin-2-yl)phenyl]carbamoyl}amino)benzoic acid (29 mg, 0.06 mmol) and piperazine (11 mg, 0.12 mmol) gave the title compound as off-white solid (15.4 mg, 46% yield). MS(ESI) m/z 555.4.

### EXAMPLE 68

### Preparation of 4-({[4-(7-ethyl-4-morpholin-4-yl-7H-pyrrolo[2,3-d]pyrimidin-2-yl)phenyl]carbamoyl}amino)-N-(2-piperidin-1-ylethyl)benzamide

Following the procedure described in Example 61, reaction of 4-({[4-(7-ethyl-4-morpholin-4-yl-7H-pyrrolo[2,3-d]pyrimidin-2-yl)phenyl]carbamoyl}amino)benzoic acid (29 mg, 0.06 mmol) and 1-(2-aminoethyl)piperidine (15 mg, 0.12 mmol) gave the title compound as off-white solid (24 mg, 67% yield). MS(ESI) m/z 597.4.

### EXAMPLE 69

### Preparation of 1-[4-(7-ethyl-4-morpholin-4-yl-7H-pyrrolo[2,3-d]pyrimidin-2-yl)phenyl]-3-{4-[(4-pyrrolidin-1-ylpiperidin-1-yl)carbonyl]phenyl}urea

Following the procedure described in Example 61, reaction of 4-({[4-(7-ethyl-4-morpholin-4-yl-7H-pyrrolo[2,3-d]pyrimidin-2-yl)phenyl]carbamoyl}amino)benzoic acid (29 mg, 0.06 mmol) and 4-(1-pyrrolidinyl)piperidine (19 mg, 0.12 mmol) gave the title compound as off-white solid (25.2 mg, 67% yield). MS(ESI) m/z 623.5.

### EXAMPLE 70

### Preparation of 1-[4-(7-ethyl-4-morpholin-4-yl-7H-pyrrolo[2,3-d]pyrimidin-2-yl)phenyl]-3-{4-[(4-ethylpiperazin-1-yl)carbonyl]phenyl}urea

Following the procedure described in Example 61, reaction of 4-({[4-(7-ethyl-4-morpholin-4-yl-7H-pyrrolo[2,3-d]pyrimidin-2-yl)phenyl]carbamoyl}amino)benzoic acid (29 mg, 0.06 mmol) and 1-ethylpiperazine (14 mg, 0.12 mmol) gave the title compound as off-white solid (23.8 mg, 68% yield). MS(ESI) m/z 583.5.

### EXAMPLE 71

### Preparation of 1-[4-(7-ethyl-4-morpholin-4-yl-7H-pyrrolo[2,3-d]pyrimidin-2-yl)phenyl]-3-[4-(thiomorpholin-4-ylcarbonyl)phenyl]urea

Following the procedure described in Example 61, reaction of 4-({[4-(7-ethyl-4-morpholin-4-yl-7H-pyrrolo[2,3-d]pyrimidin-2-yl)phenyl]carbamoyl}amino)benzoic acid (29 mg, 0.06 mmol) and thiomorpholine (12 mg, 0.12 mmol) gave the title compound as off-white solid (24.8 mg, 72% yield). MS(ESI) m/z 572.3.

### EXAMPLE 72

### Preparation of 1-[4-(1,4′-bipiperidin-1′-ylcarbonyl)phenyl]-3-[4-(7-ethyl-4-morpholin-4-yl-7H-pyrrolo[2,3-d]pyrimidin-2-yl)phenyl]urea

Following the procedure described in Example 61, reaction of 4-({[4-(7-ethyl-4-morpholin-4-yl-7H-pyrrolo[2,3-d]pyrimidin-2-yl)phenyl]carbamoyl}amino)benzoic acid (29 mg, 0.06 mmol) and 4-piperidinopiperidine (20 mg, 0.12 mmol) gave the title compound as off-white solid (23.8 mg, 62% yield). MS(ESI) m/z 637.4.

### EXAMPLE 73

### Preparation of 1-{4-[(4-cyclopentylpiperazin-1-yl)carbonyl]phenyl}-3-[4-(7-ethyl-4-morpholin-4-yl-7H-pyrrolo[2,3-d]pyrimidin-2-yl)phenyl]urea

Following the procedure described in Example 61, reaction of 4-({[4-(7-ethyl-4-morpholin-4-yl-7H-pyrrolo[2,3-d]pyrimidin-2-yl)phenyl]carbamoyl}amino)benzoic acid (29 mg, 0.06 mmol) and 1-cyclopentylpiperazine (18 mg, 0.12 mmol) gave the title compound as off-white solid (7.2 mg, 19% yield). MS(ESI) m/z 623.4.

### EXAMPLE 74

### Preparation of N-[3-(dimethylamino)propyl]-4-({[4-(7-ethyl-4-morpholin-4-yl-7H-pyrrolo[2,3-d]pyrimidin-2-yl)phenyl]carbamoyl}amino)benzamide

Following the procedure described in Example 61, reaction of 4-({[4-(7-ethyl-4-morpholin-4-yl-7H-pyrrolo[2,3-d]pyrimidin-2-yl)phenyl]carbamoyl}amino)benzoic acid (29 mg, 0.06 mmol) and 3-(dimethylamino)-1-propylamine (12 mg, 0.12 mmol) gave the title compound as off-white solid (15.4 mg, 45% yield). MS(ESI) m/z 571.4.

### EXAMPLE 75

### Preparation of 1-[4-(7-ethyl-4-morpholin-4-yl-7H-pyrrolo[2,3-d]pyrimidin-2-yl)phenyl]-3-{4-[(4-pyridin-2-ylpiperazin-1-yl)carbonyl]phenyl}urea

Following the procedure described in Example 61, reaction of 4-({[4-(7-ethyl-4-morpholin-4-yl-7H-pyrrolo[2,3-d]pyrimidin-2-yl)phenyl]carbamoyl}amino)benzoic acid (29 mg, 0.06 mmol) and 1-(2-pyridyl)piperazine (20 mg, 0.12 mmol) gave the title compound as off-white solid (3 mg, 8% yield). MS(ESI) m/z 632.4.

### EXAMPLE 76

### Preparation of 4-({[4-(7-ethyl-4-morpholin-4-yl-7H-pyrrolo[2,3-d]pyrimidin-2-yl)phenyl]carbamoyl}amino)-N-(2-pyrrolidin-1-ylethyl)benzamide

Following the procedure described in Example 61, reaction of 4-({[4-(7-ethyl-4-morpholin-4-yl-7H-pyrrolo[2,3-d]pyrimidin-2-yl)phenyl]carbamoyl}amino)benzoic acid (29 mg, 0.06 mmol) and 1-(2-aminoethyl)pyrrolidine (14 mg, 0.12 mmol) gave the title compound as off-white solid (22.6 mg, 65% yield). MS(ESI) m/z 583.4.

### EXAMPLE 77

### Preparation of 1-[4-(7-ethyl-4-morpholin-4-yl-7H-pyrrolo[2,3-d]pyrimidin-2-yl)phenyl]-3-{4-[(4-morpholin-4-ylpiperidin-1-yl)carbonyl]phenyl}urea

Following the procedure described in Example 61, reaction of 4-({[4-(7-ethyl-4-morpholin-4-yl-7H-pyrrolo[2,3-d]pyrimidin-2-yl)phenyl]carbamoyl}amino)benzoic acid (29 mg, 0.06 mmol) and 4-morpholinopiperidine (21 mg, 0.12 mmol) gave the title compound as off-white solid (26.8 mg, 70% yield). MS(ESI) m/z 639.4.

### EXAMPLE 78

### Preparation of 4-({[4-(7-ethyl-4-morpholin-4-yl-7H-pyrrolo[2,3-d]pyrimidin-2-yl)phenyl]carbamoyl}amino)-N-(2-methoxyethyl)benzamide

Following the procedure described in Example 61, reaction of 4-({[4-(7-ethyl-4-morpholin-4-yl-7H-pyrrolo[2,3-d]pyrimidin-2-yl)phenyl]carbamoyl}amino)benzoic acid (29 mg, 0.06 mmol) and 2-methoxyethylamine (9 mg, 0.12 mmol) gave the title compound as off-white solid (25.3 mg, 78% yield). MS(ESI) m/z 544.4.

The compounds in Table 1 were made by the proceeding methods.

TABLE 1

| | | MS |
|---|---|---|
| | | (ESI) |
| Example | Name | m/z |
| 79 | 1-[4-(7-isopropyl-4-morpholin-4-yl-7H-pyrrolo[2,3- | 458.5 |
| | d]pyrimidin-2-yl)phenyl]-3-pyridin-4-ylurea | |
| 80 | 1-[4-(7-isopropyl-4-morpholin-4-yl-7H-pyrrolo[2,3- | 555.5 |
| | d]pyrimidin-2-yl)phenyl]-3-[4-(4-methylpiperazin-1- | |
| | yl)phenyl]urea | |
| 81 | 1-{4-[2-(dimethylamino)ethoxy]phenyl}-3-[4-(7- | 544.4 |
| | isopropyl-4-morpholin-4-yl-7H-pyrrolo[2,3- | |
| | d]pyrimidin-2-yl)phenyl]urea | |
| 82 | 1-[4-(7-isopropyl-4-morpholin-4-yl-7H-pyrrolo[2,3- | 583.4 |
| | d]pyrimidin-2-yl)phenyl]-3-{4-[(4-methylpiperazin-1- | |
| | yl)carbonyl]phenyl}urea | |
| 83 | 1-[4-(7-isopropyl-4-morpholin-4-yl-7H-pyrrolo[2,3- | 569.5 |
| | d]pyrimidin-2-yl)phenyl]-3-[4-(piperazin-1- | |
| | ylcarbonyl)phenyl]urea | |
| 84 | 1-(4-{[4-(dimethylamino)piperidin-1- | 611.4 |
| | yl]carbonyl}phenyl)-3-[4-(7-isopropyl-4-morpholin-4- | |
| | yl-7H-pyrrolo[2,3-d]pyrimidin-2-yl)phenyl]urea | |
| 85 | N-[2-(dimethylamino)ethyl]-4-({[4-(7-isopropyl-4- | 585.4 |
| | morpholin-4-yl-7H-pyrrolo[2,3-d]pyrimidin-2- | |
| | yl)phenyl]carbamoyl}amino)-N-methylbenzamide | |
| 86 | N-[2-(dimethylamino)ethyl]-4-({[4-(7-isopropyl-4- | 571.4 |
| | morpholin-4-yl-7H-pyrrolo[2,3-d]pyrimidin-2- | |
| | yl)phenyl]carbamoyl}amino)benzamide | |
| 87 | 4-({[4-(7-isopropyl-4-morpholin-4-yl-7H-pyrrolo[2,3- | 597.4 |
| | d]pyrimidin-2-yl)phenyl]carbamoyl}amino)-N-(2- | |
| | pyrrolidin-1-ylethyl)benzamide | |
| 88 | 1-[4-(7-isopropyl-4-morpholin-4-yl-7H-pyrrolo[2,3- | 637.4 |
| | d]pyrimidin-2-yl)phenyl]-3-{4-[(4-pyrrolidin-1- | |
| | ylpiperidin-1-yl)carbonyl]phenyl}urea | |
| 89 | methyl 4-({[4-(7-isopropyl-4-morpholin-4-yl-7H- | 515.2 |
| | pyrrolo[2,3-d]pyrimidin-2- | |
| | yl)phenyl]carbamoyl}amino)benzoate | |
| 90 | 4-({[4-(7-isopropyl-4-morpholin-4-yl-7H-pyrrolo[2,3- | 501.3 |
| | d]pyrimidin-2-yl)phenyl]carbamoyl}amino)benzoic | |
| | acid | |
| 91 | 1-[4-(7-ethyl-4-morpholin-4-yl-7H-pyrrolo[2,3- | 597.5 |
| | d]pyrimidin-2-yl)phenyl]-3-(4-{[4-(1- | |
| | methylethyl)piperazin-1-yl]carbonyl}phenyl)urea | |
| 92 | 1-{4-[(4-ethylpiperazin-1-yl)carbonyl]phenyl}-3-{4-[7- | 597.5 |
| | (1-methylethyl)-4-morpholin-4-yl-7H-pyrrolo[2,3- | |
| | d]pyrimidin-2-yl]phenyl}urea | |
| 93 | 1-{4-[7-(1-methylethyl)-4-morpholin-4-yl-7H- | 611.5 |
| | pyrrolo[2,3-d]pyrimidin-2-yl]phenyl}-3-(4-{[4-(1- | |
| | methylethyl)piperazin-1-yl]carbonyl}phenyl)urea | |
| 94 | tert-butyl 4-(4-morpholin-4-yl-2-{4-[(pyridin-3- | 599.6 |
| | ylcarbamoyl)amino]phenyl}-7H-pyrrolo[2,3- | |
| | d]pyrimidin-7-yl)piperidine-1-carboxylate | |
| 95 | 4-[4-morpholin-4-yl-7-(2,2,2-trifluoroethyl)-7H- | 378.2 |
| | pyrrolo[2,3-d]pyrimidin-2-yl]aniline | |
| 96 | 1-[4-(7-ethyl-4-morpholin-4-yl-7H-pyrrolo[2,3- | 381 |
| | d]pyrimidin-2-yl)phenyl]-3-methylurea | |
| 97 | 1-(4-{5-[(dimethylamino)methyl]-7-ethyl-4-morpholin- | 438 |
| | 4-yl-7H-pyrrolo[2,3-d]pyrimidin-2-yl}phenyl)-3- | |
| | methylurea | |

### Biological Evaluation

### mTOR Kinase Assay Methods

The routine human TOR assays with purified enzyme were performed in 96-well plates by DELFIA format as follows. Enzymes were first diluted in kinase assay buffer (10 mM HEPES (pH 7.4), 50 mM NaCl, 50 mM β-glycerophosphate, 10 mM MnCl₂, 0.5 mM DTT, 0.25 μM microcystin LR, and 100 μg/mL BSA). To each well, 12 μL of the diluted enzyme were mixed briefly with 0.5 μL test inhibitor or the control vehicle dimethylsulfoxide (DMSO). The kinase reaction was initiated by adding 12.5 μL kinase assay buffer containing ATP and His6-S6K to give a final reaction volume of 25 μL containing 800 ng/mL FLAG-TOR, 100 μM ATP and 1.25 μM His6-S6K. The reaction plate was incubated for 2 hours (linear at 1-6 hours) at room temperature with gentle shaking and then terminated by adding 25 μL Stop buffer (20 mM HEPES (pH 7.4), 20 mM EDTA, 20 mM EGTA). The DELFIA detection of the phosphorylated (Thr-389) His6-S6K was performed at room temperature using a monoclonal anti-P(T389)-p70S6K antibody (1A5, Cell Signaling) labeled with Europium-N1-ITC (Eu) (10.4 Eu per antibody, PerkinElmer). The DELFIA Assay buffer and Enhancement solution were purchased from PerkinElmer. 45 μL of the terminated kinase reaction mixture was transferred to a MaxiSorp plate (Nunc) containing 55 μL PBS. The His6-S6K was allowed to attach for 2 hours after which the wells were aspirated and washed once with PBS. 100 μL of DELFIA Assay buffer with 40 ng/mL Eu-P(T389)-S6K antibody was added. The antibody binding was continued for 1 hour with gentle agitation. The wells were then aspirated and washed 4 times with PBS containing 0.05% Tween-20 (PBST). 100 μL of DELFIA Enhancement solution was added to each well and the plates were read in a PerkinElmer Victor model plate reader. Data obtained were used to calculate enzymatic activity and enzyme inhibition by potential inhibitors.

### PI3K-alpha and PI3K-gamma Fluorescence Polarization Assay Protocols

The reaction buffer was 20 mM HEPES pH 7.5, 2 mM MgCl₂, 0.05% CHAPS, and 0.01% βME (added fresh). The substrate solution was 40 μM PIP2 (diC8, Echelon, Salt Lake City Utah cat # P-4508, 1 mM in water) and 50 μM ATP in the reaction buffer. Nunc 384-well black polypropylene fluorescent plates were used for PI3K assays. The assay is run by putting 9.5 μl of freshly diluted enzyme in the reaction buffer per well, adding 0.5 μl of diluted drug or DMSO, and mixing. Then 10 μl of the substrate solution is added to each well to start the reaction. A final concentration of 20 μM PIP2 and 25 μM ATP in the reaction was used. Reactions were allowed to proceed for 30-60 minutes at room temperature. After 30-60 minutes, 20 μl of a solution of 10 nM TAMRA detector (Red detector probe-Echelon) and 2.5 μM of GST-murineGRP (1.5 mg/ml in 17% glycerol) was added per well to stop the reaction. The resulting solution was mixed well and allowed to stand for 90-110 minutes before reading plate. Assay Plates were read on Perkin-Elmer Envision plate readers with appropriate filters for Tamra [BODIPY-TMRI(1,3,4,5)P4]. Data obtained were used to calculate enzymatic activity and enzyme inhibition by inhibitor compounds. It is important to keep Red probe solutions dark. This procedure is adapted from Echelon Biosciences Inc procedure for their PI3-Kinase fluorescence polarization activity Assay kit Product number K-1100.

### In Vitro Cell Growth Assay

Cell lines used were human adenocarcinoma (LoVo), pancreatic (PC3), prostate (LNCap), breast (MDA468, MCF7), colon (HCT116), renal (HTB44 A498), and ovarian (OVCAR3) tumor cell lines. The tumor cells were plated in 96-well culture plates at approximately 3000 cells per well. One day following plating, various concentrations of inhibitors in DMSO were added to cells (final DMSO concentration in cell assays was 0.25%). Three days after drug treatment, viable cell densities were determined by cell mediated metabolic conversion of the dye MTS, a well-established indicator of cell proliferation in vitro. Cell growth assays were performed using kits purchased from Promega Corporation (Madison, Wis.), following the protocol provided by the vendor. Measuring absorbance at 490 nm generated MTS assay results. Compound effect on cell proliferation was assessed relative to untreated control cell growth. The drug concentration that conferred 50% inhibition of growth was determined as IC₅₀ (μM). IC₅₀ values of 20 nM to several μM were observed in the various tumor lines for compounds of this invention.

Table 2 shows the results of the described PI3K-α, PI3K-γ, and mTOR kinase assays.

TABLE 2

| | PI3Kα Median | PI3Kγ Median | mTOR Kinase Median |
|---|---|---|---|
| Compound | IC₅₀ (nM) | IC₅₀ (nM) | IC₅₀ (μM) |
| 1 | 80 | 752 | 0.205 |
| 2 | 43 | 338 | 0.064 |
| 3 | 830 | 10850 | 1.2 |
| 4 | 28 | 65 | <0.01668 |
| 5 | 15 | 78 | 0.0017 |
| 6 | 76 | 1677 | >0.80000 |
| 7 | 42 | 712 | >0.80000 |
| 8 | 2976 | >10000 | >3.75000 |
| 9 | 16 | 102 | 0.00295 |
| 10 | 16 | 72 | 0.00088 |
| 11 | 142 | 626 | 0.00695 |
| 12 | 11 | 36 | 0.00175 |
| 13 | 24 | 176 | 0.00775 |
| 14 | 78 | 554 | 2.15 |
| 15 | 162 | 4360 | 3.4 |
| 16 | 226 | 538 | 0.03 |
| 17 | 54 | 813 | 0.033 |
| 18 | 1397 | 2177 | 0.102 |
| 19 | 99 | 868 | 0.435 |
| 20 | 61 | 196 | 0.0035 |
| 21 | 17 | 170 | 0.00114 |
| 22 | 2 | 7 | 0.00275 |
| 23 | 30 | 370 | 0.00735 |
| 24 | 53 | 774 | 0.0525 |
| 25 | 41 | 418 | 0.033 |
| 26 | 46 | 548 | 0.0195 |
| 27 | 36 | 424 | 0.011 |
| 28 | 42 | 317 | 0.024 |
| 29 | 7 | 36 | 0.0145 |
| 30 | 14 | 245 | 0.01065 |
| 31 | 86 | 404 | 0.0255 |
| 32 | 37 | 245 | 0.023 |
| 33 | 12 | 100 | 0.0053 |
| 34 | 4 | 40 | 0.0015 |
| 35 | 12 | 100 | 0.00155 |
| 36 | 10 | 63 | 0.0008 |
| 37 | 26 | 97 | 0.00109 |
| 38 | 82 | 1526 | 0.00345 |
| 39 | 17 | 41 | 0.00195 |
| 40 | 12 | 80 | 0.00109 |
| 41 | 154 | 664 | 0.11 |
| 42 | 110 | 390 | 0.00094 |
| 43 | 570 | 1719 | 0.0039 |
| 44 | 26 | 144 | 0.0019 |
| 45 | 45 | 506 | 0.0365 |
| 46 | 14 | 104 | 0.0034 |
| 47 | 1085 | 5231 | 0.3 |
| 48 | 1196 | 3658 | 0.34 |
| 49 | 1840 | 210 | 1.45 |
| 50 | 8710 | >10000 | 0.0049 |
| 51 | 0.9 | 14 | 0.00048 |
| 52 | 1.1 | 24 | 0.0003 |
| 53 | <2.1 | 16 | 0.00057 |
| 54 | 1.9 | 16 | 0.0011 |
| 55 | <1.9 | 15 | 0.00094 |
| 56 | 2.6 | 40 | 0.00053 |
| 57 | 6 | 27 | 0.0017 |
| 58 | 2 | 11 | 0.0045 |
| 59 | 166 | 562 | 0.0047 |
| 60 | 14.5 | 120 | 0.00086 |
| 61 | <1.9 | 20 | 0.00043 |
| 62 | <2.4 | 25 | 0.00056 |
| 63 | 1.4 | 19 | 0.00175 |
| 64 | <1.7 | 13 | 0.0018 |
| 65 | 1.1 | 20 | 0.00109 |
| 66 | 3.5 | 30 | 0.0024 |
| 67 | <1.8 | 8 | 0.00099 |
| 68 | 4 | 61 | 0.00091 |
| 69 | 2.1 | 24 | 0.0008 |
| 70 | 2.5 | 21 | 0.0015 |
| 71 | 3.5 | 39 | 0.0042 |
| 72 | 3.5 | 28 | 0.0014 |
| 73 | 6.5 | 35 | 0.00335 |
| 74 | 1.9 | 32 | 0.0012 |
| 75 | 8 | 36 | 0.00565 |
| 76 | 2 | 38 | 0.00072 |
| 77 | 5.5 | 64 | 0.00175 |
| 78 | 9.5 | 118 | 0.00115 |
| 79 | 28 | 193 | 0.00115 |
| 80 | 47 | 135 | 0.0028 |
| 81 | 34 | 219 | 0.00295 |
| 82 | 3.3 | 38 | 0.0015 |
| 83 | <2.3 | 13 | 0.00064 |
| 84 | 4 | 38 | 0.0008 |
| 85 | <1.8 | 34 | 0.00061 |
| 86 | 1.7 | 50 | 0.00038 |
| 87 | 5 | 88 | 0.00055 |
| 88 | 4.3 | 38 | 0.00059 |
| 89 | 116.3 | 511 | 0.0048 |
| 90 | 25.7 | 151 | 0.00081 |
| 91 | 4 | 50 | 0.00073 |
| 92 | 8.5 | 72 | 0.00067 |
| 93 | 12 | 82 | 0.00094 |
| 94 | 11000 | >10000 | 1.35 |
| 95 | n/a | n/a | n/a |
| 96 | 140 | 1543 | 0.0049 |
| 97 | 1891 | 12000 | 0.047 |

Throughout this application, various publications are referenced. The disclosures of these publications in their entireties are hereby incorporated by reference into this application in order to more fully describe the state of the art as known to those skilled therein as of the date of the invention described and claimed herein.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A compound of the Formula I: 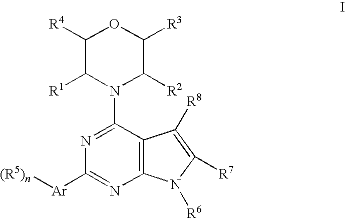
or a pharmaceutically acceptable salt thereof wherein;
R¹, R², R³, and R⁴ are each independently H or C₁-C₆alkyl-;
or either R¹ and R² or R³ and R⁴ together may form an C₁-C₃alkylene chain which, when taken together with the morpholine ring to which said chain is attached, forms a bridged, bicyclic ring, and optionally one CH₂ group in the C₁-C₃alkylene chain is replaced with —N(H)—, —N(C₁-C₆alkyl)-, —N(C₆-C₁₄aryl)-, —S—, —SO—, —S(O)₂—, or —O—;
Ar is phenyl, naphthyl, or a nitrogen-containing mono- or bicyclic heteroaryl-;
n is 0, 1, 2, or 3;
R⁵ is independently:
a) C₁-C₈acyl-,
b) C₁-C₆alkyl-, which is optionally substituted with from 1 to 3 substituents independently selected from:
i) H₂N—,
ii) (C₁-C₆alkyl)amino-,
iii) di(C₁-C₆alkyl)amino-, and
iv) C₁-C₉heterocyclyl-,
c) (C₁-C₆alkyl)amido-,
d) (C₁-C₆alkyl)carboxyl-,
e) (C₁-C₆alkyl)carbonylamido-,
f) C₁-C₆alkoxy- optionally substituted by C₁-C₆alkoxy- or C₁-C₉heteroaryl-,
g) (C₁-C₆alkoxy)carbonyl-,
h) (C₆-C₁₄aryl)oxy-,
i) C₃-C₈cycloalkyl-,
j) halo-,
k) C₁-C₆haloalkyl-,
l) C₁-C₉heterocyclyl- optionally substituted by C₁-C₆alkyl- or C₁-C₆hydroxylalkyl-,
m) heterocyclyl(C₁-C₆alkyl)- optionally substituted by C₁-C₆alkyl-,
n) hydroxyl-,
o) C₁-C₆hydroxylalkyl-,
p) C₁-C₆perfluoroalkyl-,
q) C₁-C₆perfluoroalkyl-O—,
r) R⁹R¹⁰N—,
s) C₁-C₉heterocyclyl-,
t) —CN,
u) HO₂C—,
v) R⁹R¹⁰NC(O)—,
w) C₁-C₉heterocyclyl-C(O)—,
x) R⁹C(O)NH—,
y) R⁹R¹⁰NS(O)₂—,
z) R⁹R¹⁰NC(O)NHC(O)NH—,
aa) R¹¹OC(O)NHC(O)NH—,
bb) C₁-C₆alkoxy-C₁-C₆alkylene-NH—C₁-C₆alkylene-,
cc) C₁-C₆hydroxylalkyl-NH—C₁-C₆alkylene-,
dd) amino(C₁-C₆alkyl)-NH—C₁-C₆alkylene-,
ee) di(C₁-C₆alkyl)amino-C₁-C₆alkylene-NH—C₁-C₆alkylene-,
ff) C₁-C₆hydroxylalkyl-NH—,
gg) amino(C₁-C₆alkyl)-NH—,
hh) (C₁-C₆alkyl)N-alkylamido-,
ii) R⁹R¹⁰NC(O)NH—,
jj) C₁-C₉heterocyclyl-C(O)NH—,
kk) R¹¹OC(O)NH—,
ll) R¹¹S(O)₂NH—,
mm) R¹¹S(O)₂—,
nn) —C(═N—(OR⁹))—(NR⁹R¹⁰), or
oo) O₂N—;
R⁹ and R¹⁰ are each independently H; C₁-C₆alkyl- optionally substituted with from 1 to 3 substituents independently selected from C₁-C₆alkoxy-, H₂N—, (C₁-C₆alkyl)amino-, di(C₁-C₆alkyl)amino-, C₆-C₁₄aryl-, C₁-C₉heterocyclyl- optionally substituted by C₁-C₆alkyl-, and C₁-C₉heteroaryl-; C₁-C₆alkoxy-; C₁-C₉heteroaryl- optionally substituted with from 1 to 3 substituents independently selected from C₁-C₆alkyl- optionally substituted with H₂N—, (C₁-C₆alkyl)amino-, or di(C₁-C₆alkyl)amino-, heterocyclyl(C₁-C₆alkyl)-, halogen, hydroxyl, H₂N—, O₂N—, H₂NSO₂—, HO₂C—, (C₁-C₆alkoxy)carbonyl-, (C₁-C₆alkoxy)C(O)NH—, (C₁-C₆alkyl)amino-, di(C₁-C₆alkyl)amino-, R¹⁶R¹⁷NC(O)—, R¹⁶O—, R¹⁶R¹⁷N—, R¹⁶R¹⁷NS(O)₂—, R¹⁶S(O)₂NR¹⁷—, R¹⁶R¹⁷NC(O)NH—, R¹⁶S—, R¹⁶S(O)—, R¹⁶S(O)₂—, R¹⁶C(O)—, C₁-C₉heterocyclyl- optionally substituted by C₁-C₆alkyl- or C₁-C₆hydroxylalkyl-, C₁-C₆hydroxylalkyl-, and perfluoro(C₁-C₆)alkyl-; C₁-C₆hydroxylalkyl-; C₁-C₉heterocyclyl-; C₆-C₁₄aryl- optionally substituted with from 1 to 3 substituents independently selected from C₁-C₆alkyl- optionally substituted with H₂N—, (C₁-C₆alkyl)amino-, or di(C₁-C₆alkyl)amino-, heterocyclyl(C₁-C₆alkyl)-, halogen, hydroxyl, H₂N—, O₂N—, H₂NSO₂—, HO₂C—, (C₁-C₆alkoxy)carbonyl-, (C₁-C₆alkoxy)C(O)NH—, (C₁-C₆alkyl)amino-, di(C₁-C₆alkyl)amino-, R¹⁶R¹⁷NC(O)—, R¹⁶O—, R¹⁶R¹⁷N—, R¹⁶R¹⁷NS(O)₂—, R¹⁶S(O)₂NR¹⁷—, R¹⁶R¹⁷NC(O)NH—, R¹⁶S—, R¹⁶S(O)—, R¹⁶S(O)₂—, R¹⁶C(O)—, C₁-C₉heterocyclyl- optionally substituted by C₁-C₆alkyl- or C₁-C₆hydroxylalkyl-, C₁-C₆hydroxylalkyl-, and perfluoro(C₁-C₆)alkyl-; or C₃-C₈cycloalkyl-;
or R⁹ and R¹⁰, when taken together with the nitrogen to which they are attached, form a 3- to 7-membered heterocycle wherein up to two of the carbon atoms of the heterocycle are optionally replaced with —N(H)—, —N(C₁-C₆alkyl)-, —N(C₆-C₁₄aryl)-, —S—, —SO—, —S(O)₂—, or —O—;
R¹¹ is C₁-C₆alkyl-; C₆-C₁₄aryl-; (C₆-C₁₄aryl)alkyl-, optionally substituted by NH₂; C₁-C₉heterocyclyl-; C₃-C₈cycloalkyl-; C₁-C₆hydroxylalkyl-; or C₁-C₆perfluoroalkyl-;
R¹⁶ and R¹⁷ are each independently H; C₁-C₆alkyl-; C₁-C₆alkoxy(C₂-C₆alkylene)-; (C₁-C₆alkyl)amino-C₂-C₆alkylene-; di(C₁-C₆alkyl)amino-C₂-C₆alkylene-; C₂-C₆alkenyl; C₂-C₆alkynyl; C₆-C₁₄aryl-; (C₆-C₁₄aryl)alkyl-; C₃-C₈cycloalkyl-; C₁-C₉heteroaryl- optionally substituted by CH₃NHC(O)—; (C₁-C₉heteroaryl)alkyl-; C₁-C₉heterocyclyl-; or heterocyclyl(C₁-C₆alkyl);
or R¹⁶ and R¹⁷, when taken together with the nitrogen to which they are attached, form a 3- to 7-membered heterocycle wherein up to two of the carbon atoms of the heterocycle are optionally replaced with —N(H)—, —N(C₁-C₆alkyl)-, —N(C₃-C₈cycloalkyl)-, —N(C₆-C₁₄aryl)-, —N(C₁-C₉heteroaryl)-, —S—, —SO—, —S(O)₂—, or —O— and wherein any carbon atom of the heterocycle is optionally substituted with from 1 or 2 substituents independently selected from C₁-C₆alkyl-, H₂N—, (C₁-C₆alkyl)amino-, di(C₁-C₆alkyl)amino-, and C₁-C₉heterocyclyl-;
R ⁶ is:
a) hydrogen;
b) C₁-C₆alkyl- optionally substituted with from 1 to 3 substituents independently selected from:
i) C₁-C₆alkoxy-,
ii) (C₁-C₆alkyl)amino-,
iii) di(C₁-C₆alkyl)amino-,
iv) —CHO,
v) HO₂C—, and
vi) (C₁-C₆alkoxy)carbonyl-;
c) C₁-C₆aminoalkyl- optionally substituted with a substituent selected from:
i) C₆-C₁₄aryl- optionally substituted with halogen,
ii) (C₁-C₉heteroaryl)alkyl-,
iii) (C₆-C₁₄aryl)alkyl
iv) H₂N—C₁-C₆alkylene-,
v) (C₁-C₆alkyl)amino-C₁-C₆alkylene-, or
vi) di(C₁-C₆alkyl)amino-C₁-C₆alkylene-;
d) carbonylamidoalkyl- optionally substituted with a substituent selected from:
i) halogen, or
ii) di(C₁-C₆alkyl)amino-;
e) C₃-C₈cycloalkyl-;
f) C₆-C₁₄aryl- optionally substituted with a substituent selected from:
i) HO₂C—,
ii) C₁-C₆hydroxylalkyl-,
iii) R¹²R¹³NC(O)—, or
iv) (C₁-C₆alkoxy)carbonyl-;
g) C₁-C₉heterocycle optionally substituted with from 1 to 3 substituents independently selected from:
i) C₁-C₈acyl, wherein the C₁-C₈acyl is optionally substituted with a NH₂,
ii) C₁-C₆alkyl-,
iii) (C₁-C₉heteroaryl)alkyl- wherein the ring portion of the (C₁-C₉heteroaryl)alkyl- group is optionally substituted with from 1 to 3 substituents independently selected from:
A) C₁-C₆alkylC(O)NH—,
B) halogen,
C) NH₂, and
D) C₁-C₆alkyl-,
iv) heterocyclyl(C₁-C₆alkyl)-, wherein the ring portion of the heterocyclyl(C₁-C₆alkyl) group is optionally substituted by a (C₆-C₁₄aryl)alkyl-,
v) (C₆-C₁₄aryl)alkyl-, wherein the ring portion of the (C₆-C₁₄aryl)alkyl- group is optionally substituted by 1 to 3 substituents independently selected from:
A) halogen,
B) C₁-C₆alkyl-,
C) di(C₁-C₆alkyl)amino-(C₁-C₆alkylene)-O—, and
D) C₁-C₉heteroaryl-; and
vi) (C₁-C₆alkoxy)carbonyl-;
h) heterocyclyl(C₁-C₆alkyl) optionally substituted with a substituent selected from:
i) C₁-C₆alkyl-,
ii) C₃-C₈cycloalkyl-,
iii) (C₁-C₆alkoxy)carbonyl-,
iv) C₁-C₆alkylcarboxy,
v) (C₆-C₁₄aryl)alkyl- wherein the ring portion of the (C₆-C₁₄aryl)alkyl- group is optionally substituted with a substituent selected from:
A) halogen,
B) C₁-C₉heteroaryl-, or
C) di(C₁-C₆alkyl)amino-(C₁-C₆alkylene)-O—,
vi) (C₁-C₉heteroaryl)alkyl- wherein the ring portion of the (C₁-C₉heteroaryl)alkyl- group is optionally substituted by a halogen, or
vii) C₁-C₈acyl, wherein the C₁-C₈acyl is optionally substituted with from 1 to 3 independently selected halogens,
i) (C₁-C₉heteroaryl)alkyl- wherein the ring portion of the (C₁-C₉heteroaryl)alkyl- is optionally substituted by 1 to 3 substituents independently selected from:
i) R¹²R¹³NC(O)NH—,
ii) (C₁-C₆alkoxy)carbonyl-,
iii) HO₂C—,
iv) hydroxyl, and
V) R¹²R¹³NC(O);
j) (C₆-C₁₄aryl)alkyl- wherein the ring portion of the (C₆-C₁₄aryl)alkyl- group is optionally by 1 to 3 substituents independently selected from:
i) R¹²R¹³NC(O)NH—,
ii) (C₁-C₆alkoxy)carbonyl-,
iii) HO₂C—,
iv) hydroxyl, and
v) R¹²R¹³NC(O);
k) C₁-C₆hydroxylalkyl-;
l) C₁-C₆perfluoroalkyl-; or
m) C₁-C₉heteroaryl- optionally substituted with a substituent selected from:
i) HO₂C—,
ii) C₁-C₆hydroxylalkyl-,
iii) R¹²R¹³NC(O)—, or
iv) (C₁-C₆alkoxy)carbonyl-;
R¹² and R¹³ are each independently:
a) H;
b) C₁-C₆alkyl- optionally substituted with a substituent selected from:
i) C₁-C₆alkylC(O)NH—,
ii) H₂N—,
iii) (C₁-C₆alkyl)amino-, or
iv) di(C₁-C₆alkyl)amino-,
c) C₃-C₈cycloalkyl-;
d) C₆-C₁₄aryl- optionally substituted with a substituent selected from:
i) halogen, or
ii) monocyclic C₁-C₆heterocycle wherein the monocyclic C₁-C₆heterocycle is optionally substituted with (C₁-C₆alkoxy)carbonyl-;
e) C₁-C₉heteroaryl-;
f) (C₁-C₉heteroaryl)alkyl-;
g) heterocyclyl(C₁-C₆alkyl)-;
h) (C₆-C₁₄aryl)alkyl-, wherein the chain portion of the (C₆-C₁₄aryl)alkyl- group is optionally substituted by a hydroxyl; or
i) monocyclic C₁-C₆heterocyclyl- optionally substituted with a (C₁-C₆alkoxy)carbonyl-;
or R¹² and R¹³, when taken together with the nitrogen to which they are attached, form a 3- to 7-membered heterocycle wherein up to two of the carbon atoms of the heterocycle are optionally replaced with —N(H)—, —N(C₁-C₆alkyl)-, —N(C₆-C₁₄aryl)-, —S—, —SO—, —S(O)₂—, or —O—;
R⁷ and R⁸ are each independently hydrogen; halogen; C₁-C₈acyl-; (C₁-C₆alkoxy)carbonyl-; C₁-C₆alkyl- optionally substituted with from 1 to 3 substituents independently selected from halogen, H₂N—, (C₁-C₆alkyl)amino-, di(C₁-C₆alkyl)amino-, (C₁-C₆alkyl)C(O)N(C₁-C₃alkyl)-, (C₁-C₆alkyl)carbonylamido-, HC(O)NH—, H₂NC(O)—, (C₁-C₆alkyl)NHC(O)—, di(C₁-C₆alkyl)NC(O)—, —CN, hydroxyl, C₁-C₆alkoxy-, HO₂C—, (C₁-C₆alkoxy)carbonyl-, —C(O)C₁-C₆alkyl-, C₆-C₁₄aryl-, C₁-C₉heteroaryl-, and C₃-C₈cycloalkyl-; C₂-C₆alkenyl- optionally substituted with from 1 to 3 substituents independently selected from halogen, H₂N—, —NH(C₁-C₆alkyl), di(C₁-C₆alkyl)amino-, (C₁-C₆alkyl)C(O)N(C₁-C₃alkyl)-, (C₁-C₆alkyl)carbonylamido-, HC(O)NH—, H₂NC(O)—, (C₁-C₆alkyl)NHC(O)—, di(C₁-C₆alkyl)NC(O)—, —CN, hydroxyl, C₁-C₆alkoxy-, HO₂C—, (C₁-C₆alkoxy)carbonyl-, —C(O)C₁-C₆alkyl-, C₆-C₁₄aryl-, C₁-C₉heteroaryl-, and C₃-C₈cycloalkyl-; C₂-C₆alkynyl- optionally substituted with from 1 to 3 substituents independently selected from halogen, H₂N—, —NH(C₁-C₆alkyl), di(C₁-C₆alkyl)amino-, (C₁-C₆alkyl)C(O)N(C₁-C₃alkyl)-, (C₁-C₆alkyl)carbonylamido-, HC(O)NH—, H₂NC(O)—, (C₁-C₆alkyl)NHC(O)—, di(C₁-C₆alkyl)NC(O)—, —CN, hydroxyl, —C₁-C₆alkoxy-, HO₂C—, (C₁-C₆alkoxy)carbonyl-, —C(O)C₁-C₆alkyl-, C₆-C₁₄aryl-, C₁-C₉heteroaryl-, and C₃-C₈cycloalkyl-; C₆-C₁₄aryl- optionally substituted with from 1 to 3 substituents independently selected from C₁-C₆alkyl-, halogen, haloalkyl-, hydroxyl, C₁-C₆hydroxyalkyl-, H₂N—, (C₁-C₆alkyl)amino-, di(C₁-C₆alkyl)amino-, HO₂C—, (C₁-C₆alkoxy)carbonyl-, —OC(O)—(C₁-C₆alkyl), —N—(C₁-C₆)alkylamido, H₂NC(O)—, -alkylcarboxamido and O₂N—; C₁-C₉heteroaryl- optionally substituted with from 1 to 3 substituents independently selected from C₁-C₆alkyl-, halogen, -haloalkyl-, hydroxyl, C₁-C₆hydroxylalkyl-, H₂N—, aminoalkyl-, di(C₁-C₆alkyl)amino-, HO₂C—, (C₁-C₆alkoxy)carbonyl-, —OC(O)—(C₁-C₆alkyl), —N—(C₁-C₆)alkylamido, H₂NC(O)—, -alkylcarboxamido and O₂N—; C₁-C₆perfluoroalkyl-; R¹⁴R¹⁵N; R¹⁴R¹⁵NS(O)₂—; or R¹⁴R¹⁵NC(O)—;
R¹⁴ and R¹⁵ are each independently H; C₁-C₆alkyl- optionally substituted with from 1 to 3 substituents independently selected from C₁-C₆alkoxy-, H₂N—, (C₁-C₆alkyl)amino-, di(C₁-C₆alkyl)amino-, C₆-C₁₄aryl-, C₁-C₉heterocyclyl-, and C₁-C₉heteroaryl-; C₁-C₆alkoxy-; C₁-C₉heteroaryl-; hydroxyl; C₆-C₁₄aryl- optionally substituted with from 1 to 3 substituents independently selected from C₁-C₆alkyl-, halogen, and perfluoro(C₁-C₆)alkyl-; or C₃-C₈cycloalkyl-;
or R¹⁴ and R¹⁵, when taken together with the nitrogen to which they are attached, form a 3- to 7-membered heterocycle wherein up to two of the carbon atoms of the heterocycle are optionally replaced with —N(H)—, —N(C₁-C₆alkyl)-, —N(C₆-C₁₄aryl)-, —S—, —SO—, —S(O)₂—, or —O—.

2. A compound of claim 1 wherein R¹ is H.

3. A compound of claim 2 wherein R² is H.

4. A compound of claim 3 wherein R³ is H.

5. A compound of claim 4 wherein R⁴ is H.

6. A compound of claim 5 wherein Ar is phenyl.

7. A compound of claim 6 wherein n is 1.

8. A compound of claim 7 wherein R⁵ is R⁹R¹⁰NC(O)NH—.

9. A compound of claim 8 wherein R⁹ is C₆-C₁₄aryl- substituted with R¹⁶R¹⁷NC(O)—.

10. A compound of claim 9 wherein R¹⁶ is di(C₁-C₆alkyl)amino-C₂-C₆alkylene-.

11. A compound of claim 10 wherein R¹⁶ is 2-(dimethylamino)ethyl.

12. A compound of claim 11 wherein R¹⁷ is H.

13. A compound of claim 12 wherein R¹⁰ is H.

14. A compound of claim 13 wherein R⁶ is C₁-C₆perfluoroalkyl-.

15. A compound of claim 14 wherein R⁶ is 11,1-trifluoroethyl.

16. A compound of claim 15 wherein R⁷ is H.

17. A compound of claim 16 wherein R⁸ is H.

18. A compound selected from the group consisting of:
[3-(4-morpholin-4-yl-7H-pyrrolo[2,3-d]pyrimidin-2-yl)phenyl]methanol;
3-(4-morpholin-4-yl-7H-pyrrolo[2,3-d]pyrimidin-2-yl)phenol;
2-(1H-indazol-4-yl)-4-morpholin-4-yl-7H-pyrrolo[2,3-d]pyrimidine;
1-[4-(4-morpholin-4-yl-7H-pyrrolo[2,3-d]pyrimidin-2-yl)phenyl]-3-pyridin-4-ylurea;
1-[4-(4-morpholin-4-yl-7H-pyrrolo[2,3-d]pyrimidin-2-yl)phenyl]-3-pyridin-3-ylurea;
3-{7-[2-(dimethylamino)ethyl]-4-morpholin-4-yl-7H-pyrrolo[2,3-d]pyrimidin-2-yl}phenol;
(3-{7-[2-(dimethylamino)ethyl]-4-morpholin-4-yl-7H-pyrrolo[2,3-d]pyrimidin-2-yl}phenyl)methanol;
4-{7-[2-(dimethylamino)ethyl]-4-morpholin-4-yl-7H-pyrrolo[2,3-d]pyrimidin-2-yl}aniline
1-(4-{7-[2-(dimethylamino)ethyl]-4-morpholin-4-yl-7H-pyrrolo[2,3-d]pyrimidin-2-yl}phenyl)-3-pyridin-3-ylurea;
7-[2-(dimethylamino)ethyl]-4-morpholin-4-yl-N-pyridin-3-yl-2-{4-[(pyridin-3-ylcarbamoyl)amino]phenyl}-7H-pyrrolo[2,3-d]pyrimidine-5-carboxamide;
1-(4-{7-[2-(dimethylamino)ethyl]-4-morpholin-4-yl-7H-pyrrolo[2,3-d]pyrimidin-2-yl}phenyl)-3-pyridin-2-ylurea;
1-(4-{7-[2-(dimethylamino)ethyl]-4-morpholin-4-yl-7H-pyrrolo[2,3-d]pyrimidin-2-yl}phenyl)-3-pyridin-4-ylurea;
1-(4-{7-[2-(dimethylamino)ethyl]-4-morpholin-4-yl-7H-pyrrolo[2,3-d]pyrimidin-2-yl}phenyl)-3-(4-fluorophenyl)urea;
1-[2-(dimethylamino)ethyl]-3-(4-{7-[2-(dimethylamino)ethyl]-4-morpholin-4-yl-7H-pyrrolo[2,3-d]pyrimidin-2-yl}phenyl)urea;
1-(4-{7-[2-(dimethylamino)ethyl]-4-morpholin-4-yl-7H-pyrrolo[2,3-d]pyrimidin-2-yl}phenyl)-3-[3-(dimethylamino)propyl]urea;
1-(4-{7-[2-(dimethylamino)ethyl]-4-morpholin-4-yl-7H-pyrrolo[2,3-d]pyrimidin-2-yl}phenyl)-3-ethylurea;
1-(4-{7-[2-(dimethylamino)ethyl]-4-morpholin-4-yl-7H-pyrrolo[2,3-d]pyrimidin-2-yl}phenyl)-3-methylurea;
1-(4-{7-[2-(dimethylamino)ethyl]-4-morpholin-4-yl-7H-pyrrolo[2,3-d]pyrimidin-2-yl}phenyl)-3-[2-(1H-indol-3-yl)ethyl]urea;
1-[3-({2-[3-(hydroxymethyl)phenyl]-4-morpholin-4-yl-7H-pyrrolo[2,3-d]pyrimidin-7-yl}methyl)phenyl]urea;
1-(4-{7-[3-(carbamoylamino)benzyl]-4-morpholin-4-yl-7H-pyrrolo[2,3-d]pyrimidin-2-yl}phenyl)-3-pyridin-4-ylurea;
1-{4-[7-(2,2-dimethoxyethyl)-4-morpholin-4-yl-7H-pyrrolo[2,3-d]pyrimidin-2-yl]phenyl}-3-pyridin-4-ylurea;
1-{4-[4-morpholin-4-yl-7-(2-oxoethyl)-7H-pyrrolo[2,3-d]pyrimidin-2-yl]phenyl}-3-pyridin-4-ylurea;
1-{4-[4-morpholin-4-yl-7-(2-pyrrolidin-1-ylethyl)-7H-pyrrolo[2,3-d]pyrimidin-2-yl]phenyl}-3-pyridin-4-ylurea;
1-{4-[4-morpholin-4-yl-7-(2-piperidin-1-ylethyl)-7H-pyrrolo[2,3-d]pyrimidin-2-yl]phenyl}-3-pyridin-4-ylurea;
1-[4-(7-{2-[(4-fluorophenyl)amino]ethyl}-4-morpholin-4-yl-7H-pyrrolo[2,3-d]pyrimidin-2-yl)phenyl]-3-pyridin-4-ylurea;
1-[4-(4-morpholin-4-yl-7-{2-[(pyridin-3-ylmethyl)amino]ethyl}-7H-pyrrolo[2,3-d]pyrimidin-2-yl)phenyl]-3-pyridin-4-ylurea;
1-{4-[7-(2-{[2-(dimethylamino)ethyl]amino}ethyl)-4-morpholin-4-yl-7H-pyrrolo[2,3-d]pyrimidin-2-yl]phenyl}-3-pyridin-4-ylurea;
1-(4-{7-[2-(4-methylpiperazin-1-yl)ethyl]-4-morpholin-4-yl-7H-pyrrolo[2,3-d]pyrimidin-2-yl}phenyl)-3-pyridin-4-ylurea;
1-{4-[4-morpholin-4-yl-7-(2-piperazin-1-ylethyl)-7H-pyrrolo[2,3-d]pyrimidin-2-yl]phenyl}-3-pyridin-4-ylurea;
1-{4-[7-(2-{[2-(1H-imidazol-5-yl)ethyl]amino}ethyl)-4-morpholin-4-yl-7H-pyrrolo[2,3-d]pyrimidin-2-yl]phenyl}-3-pyridin-4-ylurea;
1-(4-{7-[2-(tert-butylamino)ethyl]-4-morpholin-4-yl-7H-pyrrolo[2,3-d]pyrimidin-2-yl}phenyl)-3-pyridin-4-ylurea;
1-(4-{7-[2-(isopropylamino)ethyl]-4-morpholin-4-yl-7H-pyrrolo[2,3-d]pyrimidin-2-yl}phenyl)-3-pyridin-4-ylurea;
1-(4-{7-[2-(methylamino)ethyl]-4-morpholin-4-yl-7H-pyrrolo[2,3-d]pyrimidin-2-yl}phenyl)-3-pyridin-4-ylurea;
1-{4-[7-(2-hydroxyethyl)-4-morpholin-4-yl-7H-pyrrolo[2,3-d]pyrimidin-2-yl]phenyl}-3-pyridin-4-ylurea;
1-(4-{7-[(2,5-dioxoimidazolidin-4-yl)methyl]-4-morpholin-4-yl-7H-pyrrolo[2,3-d]pyrimidin-2-yl}phenyl)-3-pyridin-4-ylurea;
1-{4-[4-morpholin-4-yl-7-(2,2,2-trifluoroethyl)-7H-pyrrolo[2,3-d]pyrimidin-2-yl]phenyl}-3-pyridin-4-ylurea;
1-{4-[4-morpholin-4-yl-7-(2,2,2-trifluoroethyl)-7H-pyrrolo[2,3-d]pyrimidin-2-yl]phenyl}-3-pyridin-3-ylurea;
1-(4-fluorophenyl)-3-{4-[4-morpholin-4-yl-7-(2,2,2-trifluoroethyl)-7H-pyrrolo[2,3-d]pyrimidin-2-yl]phenyl}urea;
1-[4-(4-methylpiperazin-1-yl)phenyl]-3-{4-[4-morpholin-4-yl-7-(2,2,2-trifluoroethyl)-7H-pyrrolo[2,3-d]pyrimidin-2-yl]phenyl}urea;
1-[4-(hydroxymethyl)phenyl]-3-{4-[4-morpholin-4-yl-7-(2,2,2-trifluoroethyl)-7H-pyrrolo[2,3-d]pyrimidin-2-yl]phenyl}urea;
1-[2-(dimethylamino)ethyl]-3-{4-[4-morpholin-4-yl-7-(2,2,2-trifluoroethyl)-7H-pyrrolo[2,3-d]pyrimidin-2-yl]phenyl}urea;
1-(2-hydroxyethyl)-3-{4-[4-morpholin-4-yl-7-(2,2,2-trifluoroethyl)-7H-pyrrolo[2,3-d]pyrimidin-2-yl]phenyl}urea;
2-hydroxyethyl{4-[4-morpholin-4-yl-7-(2,2,2-trifluoroethyl)-7H-pyrrolo[2,3-d]pyrimidin-2-yl]phenyl}carbamate;
1-[4-(7-methyl-4-morpholin-4-yl-7H-pyrrolo[2,3-d]pyrimidin-2-yl)phenyl]-3-pyridin-3-ylurea;
5-[4-morpholin-4-yl-7-(2,2,2-trifluoroethyl)-7H-pyrrolo[2,3-d]pyrimidin-2-yl]-1H-benzimidazol-2-amine;
1-{5-[4-morpholin-4-yl-7-(2,2,2-trifluoroethyl)-7H-pyrrolo[2,3-d]pyrimidin-2-yl]-1H-benzimidazol-2-yl}-3-pyridin-3-ylurea;
N-{5-[4-morpholin-4-yl-7-(2,2,2-trifluoroethyl)-7H-pyrrolo[2,3-d]pyrimidin-2-yl]-1H-benzimidazol-2-yl}isonicotinamide;
N-methyl-5-[4-morpholin-4-yl-7-(2,2,2-trifluoroethyl)-7H-pyrrolo[2,3-d]pyrimidin-2-yl]-1H-benzimidazol-2-amine;
ethyl {5-[4-morpholin-4-yl-7-(2,2,2-trifluoroethyl)-7H-pyrrolo[2,3-d]pyrimidin-2-yl]-1H-benzimidazol-2-yl}carbamate;
methyl 4-[({4-[4-morpholin-4-yl-7-(2,2,2-trifluoroethyl)-7H-pyrrolo[2,3-d]pyrimidin-2-yl]phenyl}carbamoyl)amino]benzoate;
N-[2-(dimethylamino)ethyl]-N-methyl-4-[({4-[4-morpholin-4-yl-7-(2,2,2-trifluoroethyl)-7H-pyrrolo[2,3-d]pyrimidin-2-yl]phenyl}carbamoyl)amino]benzamide;
N-[2-(dimethylamino)ethyl]-4-[({4-[4-morpholin-4-yl-7-(2,2,2-trifluoroethyl)-7H-pyrrolo[2,3-d]pyrimidin-2-yl]phenyl}carbamoyl)amino]benzamide;
N-methyl-N-[2-(methylamino)ethyl]-4-[({4-[4-morpholin-4-yl-7-(2,2,2-trifluoroethyl)-7H-pyrrolo[2,3-d]pyrimidin-2-yl]phenyl}carbamoyl)amino]benzamide;
1-{4-[(4-methylpiperazin-1-yl)carbonyl]phenyl}-3-{4-[4-morpholin-4-yl-7-(2,2,2-trifluoroethyl)-7H-pyrrolo[2,3-d]pyrimidin-2-yl]phenyl}urea;
1-{4-[(3,3-dimethylpiperazin-1-yl)carbonyl]phenyl}-3-{4-[4-morpholin-4-yl-7-(2,2,2-trifluoroethyl)-7H-pyrrolo[2,3-d]pyrimidin-2-yl]phenyl}urea;
4-[({4-[4-morpholin-4-yl-7-(2,2,2-trifluoroethyl)-7H-pyrrolo[2,3-d]pyrimidin-2-yl]phenyl}carbamoyl)amino]-N-(2-piperidin-1-ylethyl)benzamide;
1-(4-{[4-(dimethylamino)piperidin-1-yl]carbonyl}phenyl)-3-{4-[4-morpholin-4-yl-7-(2,2,2-trifluoroethyl)-7H-pyrrolo[2,3-d]pyrimidin-2-yl]phenyl}urea;
1-{4-[2-(dimethylamino)ethoxy]phenyl}-3-{4-[4-morpholin-4-yl-7-(2,2,2-trifluoroethyl)-7H-pyrrolo[2,3-d]pyrimidin-2-yl]phenyl}urea;
methyl 4-({[4-(7-ethyl-4-morpholin-4-yl-7H-pyrrolo[2,3-d]pyrimidin-2-yl)phenyl]carbamoyl}amino)benzoate;
4-({[4-(7-ethyl-4-morpholin-4-yl-7H-pyrrolo[2,3-d]pyrimidin-2-yl)phenyl]carbamoyl}amino)benzoic acid;
N-[2-(dimethylamino)ethyl]-4-({[4-(7-ethyl-4-morpholin-4-yl-7H-pyrrolo[2,3-d]pyrimidin-2-yl)phenyl]carbamoyl}amino)-N-methylbenzamide;
N-[2-(dimethylamino)ethyl]-4-({[4-(7-ethyl-4-morpholin-4-yl-7H-pyrrolo[2,3-d]pyrimidin-2-yl)phenyl]carbamoyl}amino)benzamide;
1-[4-(7-ethyl-4-morpholin-4-yl-7H-pyrrolo[2,3-d]pyrimidin-2-yl)phenyl]-3-{4-[(4-methylpiperazin-1-yl)carbonyl]phenyl}urea;
1-(4-{[(3R,5S)-3,5-dimethylpiperazin-1-yl]carbonyl}phenyl)-3-[4-(7-ethyl-4morpholin-4yl-7H-pyrrolo[2,3-d]pyrimidin-2-yl)phenyl]urea;
1-(4-{[4-(dimethylamino)piperidin-1-yl]carbonyl}phenyl)-3-[4-(7-ethyl-4-morpholin-4-yl-7H-pyrrolo[2,3-d]pyrimidin-2-yl)phenyl]urea;
1-[4-(7-ethyl-4-morpholin-4-yl-7H-pyrrolo[2,3-d]pyrimidin-2-yl)phenyl]-3-[4-(morpholin-4-ylcarbonyl)phenyl]urea;
1-[4-(7-ethyl-4-morpholin-4-yl-7H-pyrrolo[2,3-d]pyrimidin-2-yl)phenyl]-3-[4-(piperazin-1-ylcarbonyl)phenyl]urea;
4-({[4-(7-ethyl-4-morpholin-4-yl-7H-pyrrolo[2,3-d]pyrimidin-2-yl)phenyl]carbamoyl}amino)-N-(2-piperidin-1-ylethyl)benzamide;
1-[4-(7-ethyl-4-morpholin-4-yl-7H-pyrrolo[2,3-d]pyrimidin-2-yl)phenyl]-3-{4-[(4-pyrrolidin-1-ylpiperidin-1-yl)carbonyl]phenyl}urea;
1-[4-(7-ethyl-4-morpholin-4-yl-7H-pyrrolo[2,3-d]pyrimidin-2-yl)phenyl]-3-{4-[(4-ethylpiperazin-1-yl)carbonyl]phenyl}urea;
1-[4-(7-ethyl-4-morpholin-4-yl-7H-pyrrolo[2,3-d]pyrimidin-2-yl)phenyl]-3-[4-(thiomorpholin-4-ylcarbonyl)phenyl]urea;
1-[4-(1,4′-bipiperidin-1′-ylcarbonyl)phenyl]-3-[4-(7-ethyl-4-morpholin-4-yl-7H-pyrrolo[2,3-d]pyrimidin-2-yl)phenyl]urea;
1-{4-[(4-cyclopentylpiperazin-1-yl)carbonyl]phenyl}-3-[4-(7-ethyl-4-morpholin-4-yl-7H-pyrrolo[2,3-d]pyrimidin-2-yl)phenyl]urea;
N-[3-(dimethylamino)propyl]-4-({[4-(7-ethyl-4-morpholin-4-yl-7H-pyrrolo[2,3-d]pyrimidin-2-yl)phenyl]carbamoyl}amino)benzamide;
1-[4-(7-ethyl-4-morpholin-4-yl-7H-pyrrolo[2,3-d]pyrimidin-2-yl)phenyl]-3-{4-[(4-pyridin-2-ylpiperazin-1-yl)carbonyl]phenyl}urea;
4-({[4-(7-ethyl-4-morpholin-4-yl-7H-pyrrolo[2,3-d]pyrimidin-2-yl)phenyl]carbamoyl}amino)-N-(2-pyrrolidin-1-ylethyl)benzamide;
1-[4-(7-ethyl-4-morpholin-4-yl-7H-pyrrolo[2,3-d]pyrimidin-2-yl)phenyl]-3-{4-[(4-morpholin-4-ylpiperidin-1-yl)carbonyl]phenyl}urea;
4-({[4-(7-ethyl-4-morpholin-4-yl-7H-pyrrolo[2,3-d]pyrimidin-2-yl)phenyl]carbamoyl}amino)-N-(2-methoxyethyl)benzamide;
1-[4-(7-isopropyl-4-morpholin-4-yl-7H-pyrrolo[2,3-d]pyrimidin-2-yl)phenyl]-3-pyridin-4-ylurea;
1-[4-(7-isopropyl-4-morpholin-4-yl-7H-pyrrolo[2,3-d]pyrimidin-2-yl)phenyl]-3-[4-(4-methylpiperazin-1-yl)phenyl]urea;
1-{4-[2-(dimethylamino)ethoxy]phenyl}-3-[4-(7-isopropyl-4-morpholin-4-yl-7H-pyrrolo[2,3-d]pyrimidin-2-yl)phenyl]urea;
1-[4-(7-isopropyl-4-morpholin-4-yl-7H-pyrrolo[2,3-d]pyrimidin-2-yl)phenyl]-3-{4-[(4-methylpiperazin-1-yl)carbonyl]phenyl}urea;
1-[4-(7-isopropyl-4-morpholin-4-yl-7H-pyrrolo[2,3-d]pyrimidin-2-yl)phenyl]-3-[4-(piperazin-1-ylcarbonyl)phenyl]urea;
1-(4-{[4-(dimethylamino)piperidin-1-yl]carbonyl}phenyl)-3-[4-(7-isopropyl-4-morpholin-4-yl-7H-pyrrolo[2,3-d]pyrimidin-2-yl)phenyl]urea;
N-[2-(dimethylamino)ethyl]-4-({[4-(7-isopropyl-4-morpholin-4-yl-7H-pyrrolo[2,3-d]pyrimidin-2-yl)phenyl]carbamoyl}amino)-N-methylbenzamide;
N-[2-(dimethylamino)ethyl]-4-({[4-(7-isopropyl-4-morpholin-4-yl-7H-pyrrolo[2,3-d]pyrimidin-2-yl)phenyl]carbamoyl}amino)benzamide;
4-({[4-(7-isopropyl-4-morpholin-4-yl-7H-pyrrolo[2,3-d]pyrimidin-2-yl)phenyl]carbamoyl}amino)-N-(2-pyrrolidin-1-ylethyl)benzamide;
1-[4-(7-isopropyl-4-morpholin-4-yl-7H-pyrrolo[2,3-d]pyrimidin-2-yl)phenyl]-3-{4-[(4-pyrrolidin-1-ylpiperidin-1-yl)carbonyl]phenyl}urea;
methyl 4-({[4-(7-isopropyl-4-morpholin-4-yl-7H-pyrrolo[2,3-d]pyrimidin-2-yl)phenyl]carbamoyl}amino)benzoate;
4-({[4-(7-isopropyl-4-morpholin-4-yl-7H-pyrrolo[2,3-d]pyrimidin-2-yl)phenyl]carbamoyl}amino)benzoic acid;
1-[4-(7-ethyl-4-morpholin-4-yl-7H-pyrrolo[2,3-d]pyrimidin-2-yl)phenyl]-3-(4-{[4-(1-methylethyl)piperazin-1-yl]carbonyl}phenyl)urea;
1-{4-[(4-ethylpiperazin-1-yl)carbonyl]phenyl}-3-{4-[7-(1-methylethyl)-4-morpholin-4-yl-7H-pyrrolo[2,3-d]pyrimidin-2-yl]phenyl}urea;
1-{4-[7-(1-methylethyl)-4-morpholin-4-yl-7H-pyrrolo[2,3-d]pyrimidin-2-yl]phenyl}-3-(4-{[4-(1-methylethyl)piperazin-1-yl]carbonyl}phenyl)urea;
tert-butyl 4-(4-morpholin-4-yl-2-{4-[(pyridin-3-ylcarbamoyl)amino]phenyl}-7H-pyrrolo[2,3-d]pyrimidin-7-yl)piperidine-1-carboxylate;
4-[4-morpholin-4-yl-7-(2,2,2-trifluoroethyl)-7H-pyrrolo[2,3-d]pyrimidin-2-yl]aniline;
1-[4-(7-ethyl-4-morpholin-4-yl-7H-pyrrolo[2,3-d]pyrimidin-2-yl)phenyl]-3-methylurea; and
1-(4-{5-[(dimethylamino)methyl]-7-ethyl-4-morpholin-4-yl-7H-pyrrolo[2,3-d]pyrimidin-2-yl}phenyl)-3-methylurea.

19. A compound selected from the group consisting of:
1-(4-(4-cyclopropylpiperazine-1-carbonyl)phenyl)-3-(4-(7-isopropyl-4-morpholino-7H-pyrrolo[2,3-d]pyrimidin-2-yl)phenyl)urea;
1-(4-(4-cyclopropylpiperazine-1-carbonyl)phenyl)-3-(4-(7-ethyl-4-morpholino-7H-pyrrolo[2,3-d]pyrimidin-2-yl)phenyl)urea;
1-(4-(4-cyclopropylpiperazine-1-carbonyl)phenyl)-3-(4-(4-morpholino-7-(2,2,2-trifluoroethyl)-7H-pyrrolo[2,3-d]pyrimidin-2-yl)phenyl)urea;
1-(4-(4-cyclopropylpiperazine-1-carbonyl)phenyl)-3-(4-(7-(2-(dimethylamino)ethyl)-4-morpholino-7H-pyrrolo[2,3-d]pyrimidin-2-yl)phenyl)urea;
(S)-1-(4-(3,4-dimethylpiperazine-1-carbonyl)phenyl)-3-(4-(7-isopropyl-4-morpholino-7H-pyrrolo[2,3-d]pyrimidin-2-yl)phenyl)urea;
(S)-1-(4-(3,4-dimethylpiperazine-1-carbonyl)phenyl)-3-(4-(7-ethyl-4-morpholino-7H-pyrrolo[2,3-d]pyrimidin-2-yl)phenyl)urea;
(S)-1-(4-(3,4-dimethylpiperazine-1-carbonyl)phenyl)-3-(4-(4-morpholino-7-(2,2,2-trifluoroethyl)-7H-pyrrolo[2,3-d]pyrimidin-2-yl)phenyl)urea;
(S)-1-(4-(7-(2-(dimethylamino)ethyl)-4-morpholino-7H-pyrrolo[2,3-d]pyrimidin-2-yl)phenyl)-3-(4-(3,4-dimethylpiperazine-1-carbonyl)phenyl)urea;
(R)-1-(4-(3,4-dimethylpiperazine-1-carbonyl)phenyl)-3-(4-(7-isopropyl-4-morpholino-7H-pyrrolo[2,3-d]pyrimidin-2-yl)phenyl)urea;
(R)-1-(4-(3,4-dimethylpiperazine-1-carbonyl)phenyl)-3-(4-(7-ethyl-4-morpholino-7H-pyrrolo[2,3-d]pyrimidin-2-yl)phenyl)urea;
(R)-1-(4-(3,4-dimethylpiperazine-1-carbonyl)phenyl)-3-(4-(4-morpholino-7-(2,2,2-trifluoroethyl)-7H-pyrrolo[2,3-d]pyrimidin-2-yl)phenyl)urea;
(R)-1-(4-(7-(2-(dimethylamino)ethyl)-4-morpholino-7H-pyrrolo[2,3-d]pyrimidin-2-yl)phenyl)-3-(4-(3,4-dimethylpiperazine-1-carbonyl)phenyl)urea;
1-(4-(3-(dimethylamino)pyrrolidine-1-carbonyl)phenyl)-3-(4-(7-isopropyl-4-morpholino-7H-pyrrolo[2,3-d]pyrimidin-2-yl)phenyl)urea;
1-(4-(3-(dimethylamino)pyrrolidine-1-carbonyl)phenyl)-3-(4-(7-ethyl-4-morpholino-7H-pyrrolo[2,3-d]pyrimidin-2-yl)phenyl)urea;
1-(4-(3-(dimethylamino)pyrrolidine-1-carbonyl)phenyl)-3-(4-(4-morpholino-7-(2,2,2-trifluoroethyl)-7H-pyrrolo[2,3-d]pyrimidin-2-yl)phenyl)urea;
1-(4-(7-(2-(dimethylamino)ethyl)-4-morpholino-7H-pyrrolo[2,3-d]pyrimidin-2-yl)phenyl)-3-(4-(3-(dimethylamino)pyrrolidine-1-carbonyl)phenyl)urea;
1-(4-(3-(dimethylamino)pyrrolidine-1-carbonyl)phenyl)-3-(4-(7-ethyl-4-morpholino-7H-pyrrolo[2,3-d]pyrimidin-2-yl)-3-fluorophenyl)urea;
1-(4-(7-ethyl-4-morpholino-7H-pyrrolo[2,3-d]pyrimidin-2-yl)-3-fluorophenyl)-3-(4-(piperazine-1-carbonyl)phenyl)urea;
1-(4-(7-ethyl-4-morpholino-7H-pyrrolo[2,3-d]pyrimidin-2-yl)-3-fluorophenyl)-3-(4-(thiomorpholine-4-carbonyl)phenyl)urea;
1-(4-(7-ethyl-4-morpholino-7H-pyrrolo[2,3-d]pyrimidin-2-yl)-3-fluorophenyl)-3-(4-(morpholine-4-carbonyl)phenyl)urea;
1-(4-(7-ethyl-4-morpholino-7H-pyrrolo[2,3-d]pyrimidin-2-yl)-3-fluorophenyl)-3-(4-(4-methylpiperazine-1-carbonyl)phenyl)urea;
1-(4-(7-ethyl-4-morpholino-7H-pyrrolo[2,3-d]pyrimidin-2-yl)-3-fluorophenyl)-3-(4-(4-ethylpiperazine-1-carbonyl)phenyl)urea;
1-(4-(7-ethyl-4-morpholino-7H-pyrrolo[2,3-d]pyrimidin-2-yl)-3-fluorophenyl)-3-(4-(4-isopropylpiperazine-1-carbonyl)phenyl)urea;
1-(4-(3,4-dimethylpiperazine-1-carbonyl)phenyl)-3-(4-(7-ethyl-4-morpholino-7H-pyrrolo[2,3-d]pyrimidin-2-yl)-3-fluorophenyl)urea;
1-(4-(7-ethyl-4-morpholino-7H-pyrrolo[2,3-d]pyrimidin-2-yl)-3-fluorophenyl)-3-(4-(3,3,4-trimethylpiperazine-1-carbonyl)phenyl)urea;
1-(4-(7-ethyl-4-morpholino-7H-pyrrolo[2,3-d]pyrimidin-2-yl)-3-fluorophenyl)-3-(4-(3,4,5-trimethylpiperazine-1-carbonyl)phenyl)urea;
N-(2-(dimethylamino)ethyl)-4-(3-(4-(7-ethyl-4-morpholino-7H-pyrrolo[2,3-d]pyrimidin-2-yl)-3-fluorophenyl)ureido)benzamide;
N-(2-(dimethylamino)ethyl)-4-(3-(4-(7-ethyl-4-morpholino-7H-pyrrolo[2,3-d]pyrimidin-2-yl)-3-fluorophenyl)ureido)-N-methylbenzamide;
1-(4-(7-ethyl-4-morpholino-7H-pyrrolo[2,3-d]pyrimidin-2-yl)phenyl)-3-(4-(pyridin-4-yloxy)phenyl)urea; and
5-(4-(3-(4-(7-ethyl-4-morpholino-7H-pyrrolo[2,3-d]pyrimidin-2-yl)phenyl)ureido)phenoxy)-N-methylpicolinamide.

20. A composition comprising a compound of claim 1 and a pharmaceutically acceptable carrier.

21. The composition of claim 20, wherein the pharmaceutically acceptable carrier is suitable for oral administration and the composition comprises an oral dosage form.

22. A composition comprising a compound of claim 1; a second compound selected from the group consisting of a topoisomerase I inhibitor, a MEK1/2 inhibitor, a HSP90 inhibitor, procarbazine, dacarbazine, gemcitabine, capecitabine, methotrexate, taxol, taxotere, mercaptopurine, thioguanine, hydroxyurea, cytarabine, cyclophosphamide, ifosfamide, nitrosoureas, cisplatin, carboplatin, mitomycin, dacarbazine, procarbizine, etoposide, teniposide, campathecins, bleomycin, doxorubicin, idarubicin, daunorubicin, dactinomycin, plicamycin, mitoxantrone, L-asparaginase, doxorubicin, epirubicin, 5-fluorouracil, docetaxel, paclitaxel, leucovorin, levamisole, irinotecan, estramustine, etoposide, nitrogen mustards, BCNU, carmustine, lomustine, vinblastine, vincristine, vinorelbine, cisplatin, carboplatin, oxaliplatin, imatinib mesylate, Avastin (bevacizumab), hexamethylmelamine, topotecan, tyrosine kinase inhibitors, tyrphostins, herbimycin A, genistein, erbstatin, hydroxyzine, glatiramer acetate, interferon beta-1a, interferon beta-1b, natalizumab, and lavendustin A; and a pharmaceutically acceptable carrier.

23. The composition of claim 22, wherein the second compound is Avastin.

24. A method of treating a PI3K-related disorder, comprising administering to a mammal in need thereof a compound of claim 1 in an amount effective to treat a PI3K-related disorder.

25. The method of claim 24, wherein the PI3K-related disorder is selected from restenosis, atherosclerosis, bone disorders, arthritis, diabetic retinopathy, psoriasis, benign prostatic hypertrophy, atherosclerosis, inflammation, angiogenesis, immunological disorders, pancreatitis, kidney disease, and cancer.

26. The method of claim 25, wherein the PI3K-related disorder is cancer.

27. The method of claim 26, wherein the cancer is selected from the group consisting of leukemia, skin cancer, bladder cancer, breast cancer, uterus cancer, ovary cancer, prostate cancer, lung cancer, colon cancer, pancreas cancer, renal cancer, gastric cancer, and brain cancer.

28. A method of treating an mTOR-related disorder, comprising administering to a mammal in need thereof a compound of claim 1 in an amount effective to treat an mTOR-related disorder.

29. The method of claim 28, wherein the mTOR-related disorder is selected from restenosis, atherosclerosis, bone disorders, arthritis, diabetic retinopathy, psoriasis, benign prostatic hypertrophy, atherosclerosis, inflammation, angiogenesis, immunological disorders, pancreatitis, kidney disease, and cancer.

30. The method of claim 29, wherein the mTOR-related disorder is cancer.

31. The method of claim 30, wherein the cancer is selected from the group consisting of leukemia, skin cancer, bladder cancer, breast cancer, uterus cancer, ovary cancer, prostate cancer, lung cancer, colon cancer, pancreas cancer, renal cancer, gastric cancer, and brain cancer.

32. A method of treating advanced renal cell carcinoma, comprising administering to a mammal in need thereof a compound of claim 1 in an amount effective to treat advanced renal cell carcinoma.

33. A method of treating acute lymphoblastic leukemia, comprising administering to a mammal in need thereof a compound of claim 1 in an amount effective to treat acute lymphoblastic leukemia.

34. A method of treating acute malignant melanoma, comprising administering to a mammal in need thereof a compound of claim 1 in an amount effective to treat malignant melanoma.

35. A method of treating soft-tissue or bone sarcoma, comprising administering to a mammal in need thereof a compound of claim 1 in an amount effective to treat soft-tissue or bone sarcoma.

36. A method of treating a cancer selected from the group consisting of leukemia, skin cancer, bladder cancer, breast cancer, uterus cancer, ovary cancer, prostate cancer, lung cancer, colon cancer, pancreas cancer, renal cancer, gastric cancer, and brain cancer comprising administering to a mammal in need thereof the composition of claim 23 in an amount effective to treat the cancer.

37. A method of inhibiting mTOR in a subject, comprising administering to a subject in need thereof a compound of claim 1 in an amount effective to inhibit mTOR.

38. A method of inhibiting PI3K in a subject, comprising administering to a subject in need thereof a compound of claim 1 in an amount effective to inhibit PI3K.

39. A method of inhibiting mTOR and PI3K together in a subject, comprising administering to a subject in need thereof a compound of claim 1 in an amount effective to inhibit mTOR and PI3K.

40. A method of synthesizing a compound of claim 1, comprising reacting a compound of the formula XXIII with either a reagent of the formula Ar(R⁵)ₙB(OH)₂ or a reagent of the formula Ar(R⁵)ₙSnBu₃ 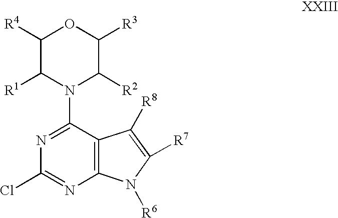
and a suitable catalyst, wherein Ar, n, and R¹-R⁸ are as defined above in formula I, thereby producing a compound of formula I: 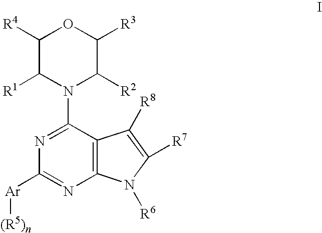
or a pharmaceutically acceptable salt thereof.

41. The method of claim 40 further comprising reacting 2,4-dichloro-7H-pyrrolo[2,3-d]pyrimidine XXI with morpholine or 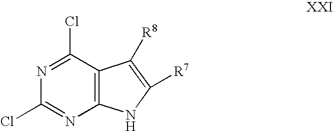
substituted or bridged morpholine V: 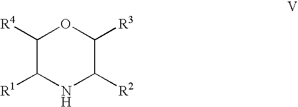
thereby proving mono chloro derivative XXII: 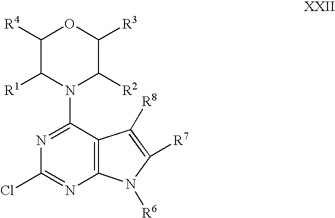
and
b) optionally alkylating the compound of formula XXII with R⁶X, thereby producing a compound of Formula XXIII when R⁶ is not H;
wherein R¹-R⁸ are as defined in claim 1, except that R⁶ is not H, and wherein X is a leaving group.
